(19) United States
(12) Patent Application Publication
(10) Pub. No.: US 2025/0367419 A1
(43) Pub. Date: Dec. 4, 2025

(54) DRUG DELIVERY SYSTEM COMPRISING AN AGENT EFFECTIVE IN THE TREATMENT OR PREVENTION OF AN ESOPHAGEAL DISEASE FOR THE APPLICATION TO ESOPHAGEAL MUCOUS MEMBRANES

(71) Applicant: EsoCap AG, Basel (CH)

(72) Inventors: Werner Weitschies, Neuenkirchen (DE); Christoph Rosenbaum, Backnang (DE); Julius Krause, Behrenhoff (DE); Friederike Brokmann, Greifswald (DE); Sabine Müller, Berlin (DE); Aileen Weide, Greifswald (DE); Bettina Appel, Greifswald (DE)

(21) Appl. No.: 18/872,782

(22) PCT Filed: Jun. 2, 2023
(86) PCT No.: PCT/EP2023/064795

§ 371 Date: Dec. 6, 2024

(30) Foreign Application Priority Data
Jun. 7, 2022 (EP) 22177671.9
Dec. 14, 2022 (EP) 22213446.2

(51) Int. Cl. A61M 31/00; A61K 9/00; A61K 9/70; A61K 31/7105

(52) U.S. Cl. CPC A61M 31/002; A61K 9/0053; A61K 9/7007; A61K 31/7105; A61M 2205/3303; A61M 2210/105

(57) ABSTRACT

The present invention relates to a drug delivery system for the application to an esophageal mucous membrane, comprising at least one sheet like, in particular film shaped, foil shaped or wafer shaped preparation comprising an agent effective in the treatment or prevention of an esophageal disease, a release mechanism, and a trigger mechanism, wherein the trigger mechanism is adapted to trigger, at a predetermined site of action the release of the sheet like preparation by the release mechanism.

### FIELD OF THE INVENTION

The present invention relates to a drug delivery system comprising an agent effective in the treatment or prevention of an esophageal disease, in particular for the application to the inner lumen including but not exclusive to the esophageal mucous membranes, and for treating esophageal diseases, in particular Barrett's esophagus, esophageal strictures and/or or esophageal cancer.

### BACKGROUND ART

Diseases of the esophagus include, for example Barrett's esophagus or Barrett's disease. Barrett's esophagus (BE) is a pre-malignant condition which is characterized by the presence of intestinal metaplasia with a specialized columnar epithelium with interspersed goblet cells that are normally present only in the small intestine and large intestine which replaces normal squamous epithelium in the distal part of the esophagus. The cells of Barrett's esophagus are classified into four categories: nondysplastic (metaplastic), low-grade dysplasia, high-grade dysplasia, and adenocarcinoma. High-grade dysplasia and early stages of adenocarcinoma may be treated by endoscopic mucosal resection, endoscopic submucosal dissection, radiofrequency ablation, or cryoablation. Later stages of adenocarcinoma may be treated with surgical resection or palliation. Those with nondysplastic mucosa are managed by annual observation with endoscopy. New guidelines of the American College of Gastroenterology 2022 recommend endoscopic eradication therapy for patients with BE and high-grade dysplasia and those with BE and low-grade dysplasia. The European Society of Gastrointestinal Endoscopy recommends endoscopic eradication in persistent (≥6 months) low-grade dysplasia. In high-grade dysplasia, the risk of developing esophageal cancer is approximately at 28% per patient-year or greater (Peters, Y., AI-Kaabi, A., Shaheen, N.J. et al. Barrett oesophagus. Nat Rev Dis Primers 5, 35 (2019).

Esophageal cancer is the sixth most common cancer worldwide, with an estimated 450,000 deaths per year. There are 2 distinct histologic types of esophageal carcinoma: squamous cell carcinoma and adenocarcinoma. Esophageal squamous cell carcinoma is more common in East Asian and Middle Eastern countries, such as China, Iran, and Turkmenistan, whereas adenocarcinoma is more prevalent in Western countries. The prevalence of adenocarcinoma has increased over the past several decades, while rates of squamous cell carcinoma have remained stable. Esophageal squamous cell carcinoma (ESCC) is among the deadliest forms of human malignancy characterized by late stage diagnosis, metastasis, therapy resistance and frequent recurrence.

Targeted drug delivery to gastrointestinal and, in particular to esophageal lumen is usually carried out via endoscopy guided sub-membranous application. Topical application of active ingredients involve drug coated esophageal stents or oral viscous drugs. Drugs which are currently under investigation involve oro-dispersible or oro-disintegrating tablets, aerosols, or gel-like drugs with higher viscosity to increase contact time.

However, topical application of active ingredients to gastrointestinal and, in particular, esophageal, membranes have some challenges. For instance, it is very difficult to locally apply high doses of a drug over a period sufficient to achieve therapeutically effective local concentrations. Possible causes of too low concentrations at the site to be treated include degeneration or deactivation of the drug by digestive secretions and enzymes, dilution effects by intestinal fluids, poor absorption, prodrugs requiring activation not available at site to be treated, and a residence time at the site of action that is too short for allowing onset of drug action effectively. Short residence times and/or too low local concentrations at the site of action are particularly a problem when using liquid or gel-like drug delivery systems. Therefore, high doses must be administered to achieve sufficient concentrations at the site to be treated. Higher administered doses of an active ingredient are usually associated with increased side effects by intestinal absorption and higher bioavailability; hence the dose of active ingredients should be kept as low as possible.

BMP2/4 inhibitors are known to be efficient in the treatment of Barrett's esophagus and are therefore effective in the prevention of esophageal adenocarcinoma. WO 2016/043577 discloses several BMP2/4 inhibitors, such as microRNAs, SMAD inhibitors, protein phosphatases which interfere with intracellular transmission of BMP2/4 signaling, as well as extracellular molecules which bind to BMP, inhibiting or enhancing BMP activity. Further, WU, J. B., “Effects of siRNA-targeting BMP-2 on the abilities of migration and invasion of human liver cancer SMMC7721 cells and its mechanism”, Cancer Gene Therapy, 2011, Vol. 18, pages 20-25, describe small interfering RNAs (siRNAs) to BMP-2. WO2018/193129 discloses several BMP2/4 inhibitors including isolated, synthetic or recombinant antibodies that efficiently inhibit the BMP2 and BMP4 signaling. Inhibition of this signaling effectively restores the normal tissue lining of the esophagus and is therefore effective in the treatment of Barrett's esophagus for prevention of esophageal adenocarcinoma. Although these inhibitors are very effective, the state-of-the-art liquid or gel-like drug delivery systems still show low concentrations at the site to be treated.

PD-1 (programmed cell death protein 1) is an immunoinhibitory receptor that is primarily expressed on activated T and B cells. Interaction with its ligands has been shown to attenuate T-cell responses both in vitro and in vivo. Blockade of the interaction between PD-1 and one of its ligands, PD-L1, has been shown to enhance tumor-specific CD8+ T-cell immunity and may therefore be helpful in clearance of tumor cells by the immune system. The role of PD-1 in cancer is established in the literature. It is known that tumor microenvironment can protect tumor cells from efficient immune destruction. PD-L1 has been shown to be expressed on a number of mouse and human tumors (and is inducible by IFN gamma on the majority of PD-Li negative tumor cell lines) and is postulated to mediate immune evasion (Iwai Y. et al., Proc. Natl. Acad. Sci. U.S.A. 99: 12293-12297 (2002); Strome S. E. et al., Cancer Res., 63: 6501-6505 (2003). Blockade of the PD-1/PD-L1 interaction could lead to enhanced tumor-specific T-cell immunity and therefore be helpful in clearance of tumor cells by the immune system and development of cancer immunotherapy.

Pembrolizumab is a humanized monoclonal antibody which targets PD-1 thereby blocking the interaction between PD-1 and its ligands PD-L1 and PD-L2. Pembrolizumab is approved for the treatment of melanoma, Hodgkin lymphoma, lung cancer, head and neck cancer, stomach cancer, urothelial cancer, cervical cancer, and breast cancer. Only recently, pembrolizumab was approved in combination with chemotherapy for treatment of advanced or metastatic esophageal cancer as first-line treatment. Pembrolizumab is administered parenterally per infusion leading to systemic exposure which is associated with severe adverse effects such as inflammation due to autoimmune reactions. Antibodies against PD-1 represent a promising target for topical/local delivery, particularly to the esophageal membrane, which might reduce adverse effects.

Paclitaxel belongs to the group of taxanes and is a chemotherapeutic agent which is currently used for the treatment of a variety of cancers including ovarian cancer, esophageal cancer, breast cancer, lung cancer, Karposi's sarcoma, cervical cancer, and pancreatic cancer. Due to its poor oral bioavailability, paclitaxel is usually administered systemically by intravenous injection and its administration is associated with severe side effects such as hair loss, heart problems, bone marrow suppression, numbness, allergic reactions, increased risk of infection, muscle pain and diarrhea. Thus, chemotherapeutic agents represent promising targets for topical/local delivery, particularly to the esophageal membrane, which might reduce the chemotherapeutic toxicity.

There is still a need for an appropriate drug delivery system, particular delivery to the esophagus, that can deliver an agent effective in the treatment or prevention of an esophageal disease for effective treatment while allowing administration of the lowest possible doses to reduce side effects and/or the delivery of stable polynucleotides or polypeptides.

### OBJECT OF THE INVENTION

It is an object of the invention to provide a drug delivery system that enables oral/topical administration of an agent effective in the treatment or prevention of an esophageal disease used for treating diseases of the esophagus with increased local efficacy.

It is a further object of the invention to provide a delivery system that allows the application of stable polynucleotides and polypeptides.

It is a further object of the invention to provide a delivery system that allows the application of an agent effective in the treatment or prevention of an esophageal disease, such as Barrett's esophagus or esophageal cancer, e.g., adenocarcinoma, esophageal junction carcinoma, or squamous cell carcinoma, at a low dose, thereby minimizing potential side effects.

The objects of the invention are achieved by the subject-matters of the independent claims. Preferred embodiments are subject of the dependent claims.

### SUBJECT MATTER CLAIMED

The present invention provides a drug delivery system for the application to an esophageal mucous membrane, comprising
at least one sheet like, in particular film shaped, foil shaped or wafer shaped preparation comprising an active pharmaceutical ingredient;
   a release mechanism; and
   a trigger mechanism, wherein
the trigger mechanism is adapted to trigger, at a predetermined site of action, the release of the preparation by the release mechanism, and wherein the release mechanism is adapted to release said preparation while moving along the esophageal mucous membrane,
wherein the drug delivery system further comprises a shell, wherein the shell contains the preparation, and wherein the shell comprises an aperture as part of the release mechanism configured to allow said preparation to leave the shell, and wherein the trigger mechanism is a holding device that is a part of or is attached to the preparation, such that the preparation is unrolled or unfolded while the dosage form moves down the esophageal mucous membrane and leaves the shell through the aperture,
characterized in that the active pharmaceutical ingredient comprises an agent effective in the treatment or prevention of an esophageal disease, preferably in combination with one or more additional active pharmaceutical ingredient(s).

In one embodiment, the agent effective in the treatment or prevention of an esophageal disease comprises or consists of an inhibiting polynucleotide, preferably an inhibiting polynucleotide in combination with a nucleic acid delivery system, an antibody, or an antiproliferative agent.

In one embodiment, the inhibiting polynucleotide is selected from the group consisting of an siRNA molecule, an antisense oligonucleotide, and an aptamer.

In one embodiment, the inhibiting polynucleotide comprises or consists of an siRNA molecule or an antisense oligonucleotide and targets an RNA transcript or a portion thereof encoding a BMP2 and/or a BMP4 polypeptide, preferably a BMP2 and/or a BMP4 polypeptide as depicted in SEQ ID NO: 15 or SEQ ID NO: 16, or wherein the inhibiting polynucleotide comprises an aptamer interfering with the activity of a BMP2 or a BMP4 polypeptide, preferably a BMP2 or a BMP4 polypeptide as depicted in SEQ ID NO: 15 or SEQ ID NO: 16.

In one embodiment, the inhibiting polynucleotide comprises or consists of an siRNA molecule or an antisense oligonucleotide and targets an RNA transcript as shown in SEQ ID NO: 20 or SEQ ID NO: 21 or a portion thereof.

In one embodiment, the siRNA molecule comprises or consists of a duplex region comprising a sequence as shown in SEQ ID NO: 17, SEQ ID NO: 18 or SEQ ID NO: 19, preferably SEQ ID NO: 18, or any other sequence comprising a sequence identity of 80% or more between the siRNA molecule and the target RNA transcript or the portion thereof wherein, preferably the target RNA transcript or the portion thereof encodes a BMP2 and/or a BMP4 polypeptide, preferably a BMP2 and/or a BMP4 polypeptide as depicted in SEQ ID NO: 15 or SEQ ID NO: 16.

In one embodiment, the siRNA molecule comprises or consists of a duplex region, wherein the duplex region comprises a sense strand and an antisense strand wherein the sense strand and the antisense strand together form the duplex region, and the antisense strand is complementary to the target RNA transcript as shown in SEQ ID NO: 20 or SEQ ID NO: 21 or a portion thereof. In one embodiment, the siRNA molecule comprises no overhang. In another embodiment, the siRNA molecule comprises an overhang of one or more nucleotide(s).

In one embodiment, the siRNA molecule comprises or consists of a duplex region, wherein the duplex region has a length of 15 to 30 base pairs, preferably of 19 to 25 base pairs.

In one embodiment, the siRNA molecule comprises or consists of BMP2-siRNA 1, BMP2-siRNA 2 or BMP2-siRNA 3, preferably BMP2-siRNA 2, as shown in the following table:

| SEQ | | | siRNA |
|---|---|---|---|
| ID | | | desig- |
| NO: | Sequence 5′ -> 3′ | Strand | nation |
| 17 | GCAGGUCUUUGCACCAAGA | Sense | BMP2 |
| | | | siRNA 1 |
| 22 | UCUUGGUGCAAAGACCUGC | Antisense | |
| 18 | GCAACAGCCAACUCGAAAU | Sense | BMP2- |
| | | | siRNA 2 |
| 23 | AUUUCGAGUUGGCUGUUGC | Antisense | |
| 19 | GCUGUACCUUGACGAGAAU | Sense | BMP2- |
| | | | siRNA 3 |
| 24 | AUUCUCGUCAAGGUACAGC | Antisense | |

wherein each of the above sequences in the table comprises an overhang of two nucleotides dTdT (deoxythymidine) or UU (uridine) attached to the 3′ end of each strand.

In one embodiment, the nucleic acid delivery system is selected from the group consisting of a liposome, a lipid double layer, a micelle, an emulsion, a cationic polymer, and a nanoparticle, preferably lipid nanoparticle or a polymer nanoparticle.

In one embodiment, the antibody or binding fragment thereof is an isolated or recombinant or synthetic antibody or a binding fragment thereof.

In one embodiment, the antibody or a binding fragment thereof binds within, preferably binds to an epitope consisting of:
a) residues 10-17, 45-56, and 69 of BMP4 (SEQ ID NO: 1),
b) residues 24-31, 57-68, 70-72, 89, 91, 101, 103, 104 and 106 of BMP4 (SEQ ID NO:1), or
c) residues 34, 35, 39, 86-88, 90, 97, 98, 100, 102 and 109 of BMP4 (SEQ ID NO:1)

wherein preferably binding of the antibody or a fragment thereof is determined by epitope binning (surface plasmon resonance (SPR) sandwich cross-binding) and/or HADDOCK modelling.

In one embodiment, the antibody or a binding fragment thereof binding within residues 10-17, 45-56, and 69 of BMP4 specifically binds to at least Lys12, Arg15, Asp46, and Pro50 of BMP4 (SEQ ID NO:1), or
the antibody or a binding fragment thereof binding within residues 24-31, 57-68, 70-72, 89, 91, 101, 103, 104 and 106 of BMP4 specifically binds to at least Asp30, Trp31, Leu66 and Lys101 of BMP4 (SEQ ID NO: 1), or
the antibody or a binding fragment thereof binding within residues 34, 35, 39, 86-88, 90, 97, 98, 100, 102 and 109 of BMP4 specifically binds to at least Ala34, Gln39, Ser88, Leu90 and Leu100 of BMP4 (SEQ ID NO: 1),
wherein preferably specific binding of the antibody or a fragment thereof is determined by epitope binning (surface plasmon resonance (SPR) sandwich cross-binding) and/or HADDOCK modelling.

In one embodiment, the antibody or a binding fragment thereof binding to at least Lys12, Arg15, Asp46, and Pro50 of BMP4 comprises a heavy chain CDR1 consisting of the amino acid sequence of SEQ ID NO: 2 or a sequence not differing more than 1 amino acid thereof, a heavy chain CDR2 consisting of the amino acid sequence of SEQ ID NO: 3, or a sequence not differing more than 1 amino acid thereof, and a heavy chain CDR3 consisting of the amino acid sequence of SEQ ID NO: 4 or a sequence not differing more than 1 amino acid thereof, or
the antibody or a binding fragment thereof binding to at least Asp30, Trp31, Leu66 and Lys101 of BMP4 comprises a heavy chain CDR1 consisting of the amino acid sequence of SEQ ID NO: 5 or a sequence not differing more than 1 amino acid thereof, a heavy chain CDR2 consisting of the amino acid sequence of SEQ ID NO: 6, or a sequence not differing more than 1 amino acid thereof, and a heavy chain CDR3 consisting of the amino acid sequence of SEQ ID NO: 7 or a sequence not differing more than 1 amino acid thereof, or
the antibody or a binding fragment thereof binding to at least Ala34, Gln39, Ser88, Leu90 and Leu100 of BMP4 comprises a heavy chain CDR1 consisting of the amino acid sequence of SEQ ID NO: 8 or a sequence not differing more than 1 amino acid thereof, a heavy chain CDR2 consisting of the amino acid sequence of SEQ ID NO: 9, or a sequence not differing more than 1 amino acid thereof, and a heavy chain CDR3 consisting of the amino acid sequence of SEQ ID NO: 10 or a sequence not differing more than 1 amino acid thereof.

In one embodiment, the antibody or a binding fragment thereof binding to at least Lys12, Arg15, Asp46, and Pro50 of BMP4 comprises the amino acid sequence of SEQ ID NO: 11 or a sequence which is at least 70%, preferably 80, 85, 90, 91, 92, 93, 94, 95, 96, 97, 98, 99 or 100% identical thereto, or
the antibody or a binding fragment thereof binding to at least Asp30, Trp31, Leu66 and Lys101 of BMP4 comprises the acid sequence of SEQ ID NO: 12 or a sequence which is at least 70%, preferably 80, 85, 90, 91, 92, 93, 94, 95, 96, 97, 98, 99 or 100% identical thereto, or
the antibody or a binding fragment thereof binding to at least Ala34, Gln39, Ser88, Leu90 and Leu100 of BMP4 comprises the acid sequence of SEQ ID NO: 13 or a sequence which is at least 70%, preferably 80, 85, 90, 91, 92, 93, 94, 95, 96, 97, 98, 99 or 100% identical thereto.

In one embodiment, the antibody or a binding fragment thereof is a single chain antibody.

In one embodiment, the active pharmaceutical ingredient comprises two different antibodies or binding fragments thereof.

In one embodiment, the antibody or antibody fragment binds to PD-1, preferably human PD-1.

In one embodiment, the antibody or antibody fragment which binds to PD-1, preferably human PD-1, comprises:
a. at least one CDR (complementary determining region) selected from the group consisting of SEQ ID NOs: 30, 31, 32, 36, 37 and 38, or a variant of any said sequence; and/or
b. at least one a CDR selected from the group consisting of SEQ ID NOs: 33, 34, 35, 39, 40 and 41, or a variant of any said sequence.

In one embodiment, the antibody or antibody fragment which binds to PD-1, preferably human PD-1, comprises:
a. light chain CDRs SEQ ID NOs: 30, 31 and 32, or variants of any said sequences; and/or heavy chain CDRs SEQ ID NOs: 33, 34 and 35, or variants of any said sequences; or
b. light chain CDRs SEQ ID NOs: 36, 37 and 38 or variants of any said sequences; and/or heavy chain CDRs SEQ ID NOs: 39, 40 and 41 or variants of any said sequences.

In one embodiment, the antibody or antibody fragment which binds to PD-1, preferably human PD-1, comprises:
a. a heavy chain variable region comprising an amino acid sequence selected from the group consisting of:
i. SEQ ID NO: 26 or a variant thereof;
ii. SEQ ID NO: 28 or a variant thereof;
iii. amino acid residues 20 to 139 of SEQ ID NO: 42 or a variant thereof; and
iv. an amino acid sequence having at least 50%, 60%, 70%, 75%, 80%, 85%, 90%, 95%, 99% sequence identity to amino acid residues 20 to 139 of SEQ ID NO: 42;
and further comprises
b. a light chain variable region comprising an amino acid sequence selected from the group consisting of:
i. SEQ ID NO: 27 or a variant thereof;
ii. SEQ ID NO: 29 or a variant thereof;
iii. amino acid residues 20 to 130 of SEQ ID NO: 44 or a variant thereof;
iv. amino acid residues 20 to 130 of SEQ ID NO: 45 or a variant thereof;
v. amino acid residues 20 to 130 of SEQ ID NO: 46 or a variant thereof; and
vi. an amino acid sequence having at least 50%, 60%, 70%, 75%, 80%, 85%, 90%, 95%, 99% sequence identity to amino acid residues 20 to 130 of SEQ ID NO: 44, 45 or 46.

In one embodiment, the antibody or antibody fragment which binds to PD-1, preferably human PD-1, comprises:
a. a heavy chain comprising an amino acid sequence selected from the group consisting of:
i. amino acid residues 20 to 466 of SEQ ID NO: 43 or a variant thereof, and
ii. amino acid residues 20 to 469 of SEQ ID NO: 47 or a variant thereof; and
b. a light chain comprising an amino acid sequence selected from the group consisting of:
i. amino acid residues 20 to 237 of SEQ ID NO: 48 or a variant thereof;
ii. amino acid residues 20 to 237 of SEQ ID NO:49 or a variant thereof, and
iii. amino acid residues 20 to 237 of SEQ ID NO: 50 or a variant thereof.

In one embodiment, the antibody or antibody fragment which binds to PD-1, wherein the antibody or antibody fragment:
a. binds human PD-1 with a KD of about 100 pM or lower;
b. binds human PD-1 with a KD of about 30 pM or lower;
c. binds to human PD-1 with about the same KD as an antibody having a heavy chain comprising the amino acid sequence of SEQ ID NO: 43 and a light chain comprising the amino acid sequence of SEQ ID NO: 44;
d. binds to human PD-1 with about the same KD as an antibody having a heavy chain comprising the amino acid sequence of SEQ ID NO: 43 and a light chain comprising the amino acid sequence of SEQ ID NO: 45;
e. binds to human PD-1 with a k_{assoc} of about 7.5×10⁵ 1/M·s or faster;
f. binds to human PD-1 with a k_{assoc} of about 1×10⁶ 1/M·s or faster;
g. binds to human PD-1 with a k_{dissoc} of about 2×10⁻⁵ 1/s or slower;
h. binds to human PD-1 with a k_{dissoc} of about 2.7×10⁻⁵ 1/s or slower;
i. binds to human PD-1 with a k_{dissoc} of about 3×10⁻⁵ 1/s or slower; and/or
j. blocks binding of human PD-L1 or human PD-L2 to human PD-1 with an IC₅₀ of about 1 nM or lower.

In one embodiment, the antiproliferative agent is selected from the group consisting of a taxane; a pyrimidine analogue, and a platinum-based agent.

In one embodiment, the antiproliferative agent is a taxane selected from the group consisting of paclitaxel ((2α,4α,5β,7β,10β,13α)-4,10-Bis(acetyloxy)-13-{[(2R,3S)-3-(benzoylamino)-2-hydroxy-3-phenylpropanoyl]oxy}-1,7-dihydroxy-9-oxo-5,20-epoxytax-11-en-2-yl-benzoat), docetaxel ((2R,3S)-4-Acetoxy-2a-benzyloxy-13-[3-(N-tert-butoxycarbonyl)amino-2-hydroxy-3-phenyl]propionyl-5β,20-epoxy-1,7β,10β-trihydroxy-9-oxotax-11-en-13α-ylester; Taxotere®), and cabazitaxel (Jevanta®), preferably paclitaxel.

In one embodiment, the antiproliferative agent is a pyrimidine analogue, wherein the pyrimidine analogue is an uracil analogue, preferably 5-flurouracil or capecitabin.

In one embodiment, the antiproliferative agent is platinum-based agent, wherein the platinum-based agent is selected from the group consisting of cisplatin ((SP-4-2)-Diammindichloridoplatin(II); DDP) or a salt thereof, carboplatin (Diamminplatin(II)-cyclobutan-1,1-dicarboxylat) or a salt thereof, nedaplatin (Aqupla®) or a salt thereof, and oxaliplatin (Pt-(Oxalato)-trans-I-diaminocyclohexan) or a salt thereof. The platinum-based agent might be further selected from the group consisting of triplatin tetranitrate (BBR3464) or a salt thereof, phenanthriplatin (cis-[Pt(NH₃)₂-(phenanthridine)Cl]NO₃) or a salt thereof, picoplatin or a salt thereof and satraplatin ((OC-6-43)-Bis(acetato-O)ammindichloro(cyclohexylamin)platin; JM216) or a salt thereof.

In one embodiment, the drug delivery system according to the invention is for use in therapy.

In one embodiment, the drug delivery system according to the invention is for use in the treatment or prevention of an esophageal disease.

In one embodiment, the drug delivery system according to the invention is for use in the treatment or prevention of an esophageal disease which is caused or related to a defect in the immune system. In one embodiment, the drug delivery system according to the invention is for use in the treatment or prevention of cancer.

In one embodiment, the drug delivery system according to the invention is for use in the treatment or prevention of an esophageal disease, such as esophageal cancer.

In one embodiment, the drug delivery system according to the invention is for use in the treatment or prevention of Barrett's esophagus, esophageal stricture, and/or esophageal cancer, such as adenocarcinoma, esophageal junction carcinoma, or squamous cell carcinoma.

In one embodiment, the drug delivery system according to the invention is for use in diagnosis, preferably wherein the active pharmaceutical ingredient is in combination with a diagnostic marker.

In one embodiment, the drug delivery system according to the invention is used for in vitro diagnosis, preferably wherein the active pharmaceutical ingredient is in combination with a diagnostic marker.

In one embodiment diagnosis comprises monitoring the cellular uptake of the active pharmaceutical ingredient, monitoring the route of the active pharmaceutical ingredient in a tissue or organ, or monitoring of a treatment success, e.g., tumor size.

### DETAILED DESCRIPTION

Unless otherwise defined, all technical and scientific terms used herein have the same meaning as commonly understood by one of ordinary skill in the art to which this disclosure belongs.

It is noteworthy that the use of the undefined article “a” or “an” means “one or more”.

Thus, for example, the term “an esophageal disease” includes the incorporation of “one” and “more than one” esophageal disease(s).

The term “comprising” or “comprises” as used herein means “including, but not limited to”. The term is intended to be open-ended, to specify the presence of any stated features, elements, integers, steps, or components, but not to preclude the presence of addition of one or more other features, elements, integers, steps, components, or groups thereof. The term “comprising” or “comprises” thus includes the more restrictive terms “consisting of” and “consisting essentially of”. In one embodiment, the term “comprising” or comprises” as used throughout the application and in particular within the claims may be replaced by the term “consisting of” or “consisting essentially of”.

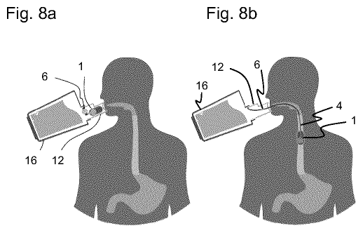

A drug delivery system comprising a pharmaceutical preparation, however with different active pharmaceutical ingredients and its application is described in PCT/EP2015/002601, which is incorporated by reference herein in full, in particular regarding the embodiment according to FIGS. 8a, 8b, 8c of PCT/EP2015/002601. Stated differently, the size, shape and composition of the shell, the aperture, the release and trigger mechanism and the holding device are at least to a significant extent already described in said reference.

The drug delivery system as described in the PCT/EP2015/002601 is designed such that it comprises at least one sheet like, in particular film shaped, foil shaped or wafer shaped preparation comprising an active pharmaceutical ingredient, a release mechanism, and a trigger mechanism, wherein the trigger mechanism is adapted to trigger, at a predetermined site of action, in particular of the gastrointestinal tract the release of the sheet like preparation by the release mechanism. From the embodiment according to FIGS. 8a, 8b, 8c of PCT/EP2015/002601, the dosage form is known to have an elongated, strip-shaped preparation, which comprises the active pharmaceutical ingredient. The preparation is capable to be arranged in a compact condition and in an expanded condition. The dosage form has a capsule device, e.g., a shell, comprising a hollow space for accommodating the compacted preparation, the capsule device has an aperture, and a first end of the preparation extends, in the compact condition, through the aperture such that the preparation can be pulled out of the hollow space into the surrounding area of the capsule thereby transferring the preparation from the compact condition to the expanded condition.

The drug delivery system according to the present invention is orally administered and improves the local availability of the agent effective in the treatment or prevention of an esophageal disease contained in the preparation. This contrasts with conventional orally administration systems, such as tablets or capsules, which are delivered via gastro-intestinal absorption into the blood circulation only to the site/location to be treated.

The local availability is improved, because the agent effective in the treatment or prevention of an esophageal disease is provided in a sheet-like, in particular film-shaped, foil-shaped, wafer-shaped, or strip shaped preparation. This advantageously allows releasing the sheet-like preparation (and the agent effective in the treatment or prevention of an esophageal disease being present therein) directly onto the site/location to be treated (treatment site), e.g., an esophageal mucous membrane. Thereby, preferably a large area of the sheet like preparation is exposed to the mucous membrane, i.e., to the esophageal mucous membranes, in particular to the inner lumen including but not exclusive to the esophageal mucous membranes. Upon exposure to the mucous membrane the sheet-like preparation releases the agent effective in the treatment or prevention of an esophageal disease. Further, the preferably direct contact between the mucous membrane and the preparation results in an effective action of the agent effective in the treatment or prevention of an esophageal disease at the treatment site. Due to the direct delivery of the agent to the treatment site, less agent is required resulting in reduced systemic bioavailability and reduced concentrations at neighboring, e.g., healthy areas as compared to the use of conventional preparations, such as suspensions or solutions. Further, the effective action of the agent effective in the treatment or prevention of an esophageal disease through the membranes of well vascularized resorption sites of the gastrointestinal tract, such as the small intestine or buccal, reduces side effects. The direct delivery to the treatment site further allows to lower the dose of the agent effective in the treatment or prevention of an esophageal disease contained in the preparation, thereby advantageously further reducing side effects. Thus, the drug delivery system of the present invention is particularly useful for the delivery of all agents whose therapeutic use is limited due to a high systemic toxicity or a low systemic availability, e.g., due to a high first pass effect or loss of/impairment of activity by passaging the gastrointestinal tract.

The drug delivery system according to the invention further advantageously allows a relatively simple and discrete handling as well as a simple, particularly space-saving storage. The agent effective in the treatment or prevention of an esophageal disease, which is comprised in the drug delivery system according to the invention, have an improved stability, e.g., at high heat and humidity, when compared to solutions and gels. Usually there is no free water left in the drug delivery system according to the invention, which further improves the stability and reduces the risk of the composition becoming e.g., moldy, or otherwise unusable. Additional additives, such as preserving agents or other stabilizers, can be avoided, which is advantageous because it is known that such additives can cause allergies or further side effects.

Also, the destruction of the active pharmaceutical ingredient before it reaches the predetermined site of action, e.g., by gastric acid and/or digestive enzymes, is advantageously minimized by a drug delivery system according to the invention.

### Release Mechanism

A release mechanism relates to a mechanism which expands and releases the sheet-like preparation from a capsule device, e.g., a shell. The shell contains the sheet-like preparation in a compact form. The release mechanism releases the preparation from the shell after a trigger mechanism has initiated the release. The release of the sheet-like preparation by the release mechanism preferably takes place by pulling the preparation at least partially out of the shell. Therefore, the sheet like preparation is adapted, such that the sheet like preparation is expandable to a predetermined extent by the release mechanism. For example, the shell contains the preparation in a folded form and the release mechanism expands the preparation from its compact, e.g., from a folded form, into its expanded, e.g., unfolded form. The release mechanism therefore causes an unfolding of the preparation. In the compact form, the preparation has a smaller spatial extent, e.g., the preparation is lumped together, coiled, or winded or brought into a smaller spatial format in another way. This also allows to provide a small dosage form, i.e., a small shell, which makes the especially oral intake of the drug delivery system more convenient for a patient. In its expanded form, the surface area of the sheet like preparation is increased by the expansion, e.g., by the unfolding of the sheet like preparation, in particular the surface area of the preparation containing the agent effective in the treatment or prevention of an esophageal disease is increased. Preferably, the surface area of the preparation, in particular the surface area, which contains the agent effective in the treatment or prevention of an esophageal disease, and which contacts the esophageal mucous membrane, is in the order of the surface area of esophageal mucous membrane. The release of the preparation occurs while the shell moves down the esophageal mucous membrane. For example, during a patient swallows the dosage form, the preparation is released from the shell through an aperture. The shell therefore comprises an aperture as part of the release mechanism, configured to allow the preparation to leave the shell.

### Aperture

In this respect the aperture forms an opening in the shell, i.e., in the capsule device. In a preferred embodiment of the drug delivery system the aperture is formed as a slit. A slit is arranged such that the sheet-like preparation is released from the shell through the aperture. Such a slit may be embodied in different arrangements and configurations. Such an aperture is described in, for example, in EP21175427.0, EP21175436.1, PCT/EP2015/002601 and PCT/EP2020/056934, which are incorporated by reference herein in full, regarding the capsule device and the aperture.

### Trigger Mechanism/Holding Device

The drug delivery system comprises a trigger mechanism, wherein the trigger mechanism is adapted to trigger, at a predetermined site of action, the release of the sheet like preparation by the release mechanism, wherein the trigger mechanism is a holding device that is part of or is attached to the preparation.

Preferably, the preparation comprises the holding device, further preferably, the preparation comprises the holding device at one end of the preparation, which, in particular protrudes out of the shell through the aperture. Upon fixation of the holding device, the preparation can be withdrawn from the capsule device by a pulling movement and/or force. Fixation of the holding device is obtained by preferably connecting the holding device to a retainer. Such a retainer can be a string member, as for example, a cord, string, or tether. In a preferred embodiment, the holding device is connected to one end of the preparation and to one end of the cord, whereas the other end of the cord is secured to an applicator, e.g., to a holder of the applicator.

Preferably, the holding device is attached to the sheet like preparation. Thereby the retainer, i.e., the string member or a part of the string member form the holding device. For example, the one end of the cord which is connected to the preparation forms the holding device.

Alternatively, the holding device is adapted to be fixed in the oral cavity or the holding device is adapted to be held in hand during administration of the drug delivery system, such that the preparation is unrolled and or unfolded while the dosage form moves down the esophageal mucous membrane and leaves the shell through the aperture.

In a preferred embodiment, a part of the string member is connected to an end portion of the preparation, which protrudes from the aperture of the capsule device. Thereby the holding device is formed by the protruding end portion of the preparation and the string member being connected to it and the further part of the string member acts as a retainer, to retain the holding device from moving while swallowing the preparation, thereby creating a pulling force which acts onto the preparation, and which pulls the preparation out of the capsule device while the capsule device moves down the esophagus.

It is to be understood that the terms “site of action” and “application site” as used herein are used interchangeably. In this regard, it is also to be understood that “site of action” and “site of application” refer to the predetermined location of release of the preparation. Moreover, an agent effective in the treatment or prevention of an esophageal disease, which is released at the “site of action” respectively “application site” may exert its actual biochemical effect also at another location of the body or at another site of a biochemical cycle, e.g., at or after metabolization by the liver or reaching of the agent at its target molecule. “Site of action” and “application site” as used herein do not necessarily refer to the location of the biochemical, medical effect of the active pharmaceutical ingredient.

### Capsule Device/Shell

The drug delivery system according to the present invention further comprises a shell, wherein the shell contains the at least one sheet-like, in particular film-shaped, foil-shaped, or wafer-shaped preparation comprising the agent effective in the treatment or prevention of an esophageal disease, and wherein the shell comprises the aperture as part of the release mechanism configured to allow said preparation to leave the shell, such that the preparation is unrolled or unfolded while the dosage form moves down the esophageal mucous membrane and leaves the shell through the aperture. The shell may further be prepared such that it protects the preparation against an unwanted release. The shell is a capsule device and, in particular, has the shape of a capsule.

In preferred embodiment, the shell comprises a first halve-capsule shell and a second halve capsule shell, and the capsule device is formed by sliding the first halve-capsule shell into the second halve-capsule shell to a joined position, such that the aperture is formed in the joined position by the second halve capsule shell overlapping a cross section of an opening, which is located in the first halve-capsule shell.

In a further embodiment, the two capsule-halves are telescoped into each other, whereas the opening of the first halve-capsule shell is covered by a further provided overlapping wall part, e.g., a patch or a tape, which is attached to the first and or second halve capsule shell.

In an alternative embodiment, the capsule halves are shaped like two nutshells and positioned on top of each other to form the capsule. The aperture is formed by a cutout, particularly at the edge of one of the two shells. Alternatively, cutouts can be formed on the edges of both halves, which when positioned and aligned on top of each other form the aperture.

In a preferred embodiment of the drug delivery system according to the present invention the shell is made from a material that is selected from the group comprising hard gelatin, polymers, thermoplastics as e.g., Eudragit or the like. In this regard, in particular, materials can be beneficial that have been successfully tested, used and/or authorized already, e.g., for oral dosage forms.

Such a capsule device or shell is further described, for example, in EP21175427.0, EP21175436.1 and PCT/EP2020/056934, which are incorporated by reference herein in full, with regard to the capsule device.

### The Conditions to be Treated

The drug delivery system described herein is for use in therapy. In one embodiment, it is adapted for the treatment and prevention of esophageal diseases, preferably an esophageal disease which is caused or related to a defect in the immune system. In one embodiment, it is adapted for the treatment and prevention of cancer, preferably esophageal cancer. As used herein, “esophageal cancer” refers to cancer that starts or is present in the esophagus, including but not limited to squamous cell carcinoma, esophageal junction carcinoma, particularly the gastroesophageal junction, and adenocarcinoma. In one embodiment, it is adapted for the treatment and prevention of Barrett's esophagus. In one embodiment, it is adapted for the treatment and prevention of esophageal adenocarcinoma. In one embodiment, it is adapted for the treatment and prevention of esophageal squamous cell carcinoma. In one embodiment, it is adapted for the treatment and prevention of esophageal strictures.

Within the subject application, the term “treatment and/or prevention” includes any way of ameliorating a certain condition to be treated or preventing the condition to be treated to occur. It also includes the prevention of a worsening of the condition and minimizing the severity of the condition.

Esophageal disease may be any disease or disorder interfering with the function or structure of the esophagus.

Esophageal diseases might comprise chronic inflammatory states that can progress through a series of transformative dysplastic states before tumor development which may also be caused by combined acid and bile reflux and may be related to a defect in the immune system. Esophageal disease includes also but is not limited to refractory esophageal disease, e.g., after a first line treatment. For example, refractory esophageal disease relates to esophageal disease which has been unsuccessfully or insufficiently treated, i.e., symptoms specific for the esophageal disease persist despite treatment. Refractory esophageal disease may also refer to relapse after treatment. Thus, in one embodiment the present invention relates to the treatment of refractory esophageal disease, in the sense of a second line treatment. For example, surgical or ablative therapies in the treatment of cancer might require a post treatment or second-line treatment with a chemotherapeutic agent, sometimes in combination with radiotherapy, to ensure complete destruction of cancerous tissue. Thus, the present invention also comprises the second-line treatment of an esophageal disease such as cancer.

In another embodiment the present invention relates to a first line treatment or pre-treatment, such as a preventive treatment or adjuvant chemotherapy followed by surgical therapy, of an esophageal disease. For example, surgical or ablative therapies in the treatment of cancer might require a first-line treatment or pre-treatment, e.g., neoadjuvant therapy, with a chemotherapeutic agent, sometimes in combination with radiotherapy, to decrease the size of a tumor to a size which is operable. Thus, the present invention also comprises the first-line treatment or pre-treatment, e.g., neoadjuvant therapy, of an esophageal disease such as cancer.

Preferred esophageal diseases comprise esophageal diseases, which are caused or related to a defect in the immune system. For example, the esophageal disease may be caused or related to a decreased proliferation or activity of a cell of the immune system thereby interfering with the esophageal homeostasis and promoting the development and/or progression of cancer, e.g., due to escape mechanisms of a cancer cell from the immune surveillance.

One treatment option of cancer is ablation of affected tissue or surgery as stand-alone therapy or in combination with chemotherapy or radiation therapy, e.g., as neoadjuvant therapy. “Neoadjuvant therapy” as used herein refers to chemotherapy and/or radiotherapy which precedes ablation or surgery and aims at a reduction of the tumor size, preferably to an operable size. Ablative therapies or surgery may be accompanied by chemotherapy in the postoperative phase to avoid spreading of the tumor and/or the formation of metastases. Alternatively, particularly in unresectable cancer, chemotherapy as stand-alone therapy or in combination with radiotherapy (definitive chemoradiotherapy) is the treatment of choice.

Independent of the time point within the treatment regimen, chemotherapy is usually administered systemically by parenteral administration which is associated with high toxicity for the patient. Thus, there is a need for novel treatment options for cancer with reduced toxicity.

Barrett's esophagus is a condition in which the normal multi-layered squamous epithelium is substituted by a (specialized) columnar epithelium (i.e., intestinal, or other columnar type of metaplasia). This process is assumed to be the result of longstanding gastro-esophageal reflux disease and is most prevalent in middle aged, Caucasian males. In particular, the specialized intestinal type of columnar metaplasia confers a significantly increased risk for the development of esophageal adenocarcinoma. Esophageal adenocarcinoma is a highly malignant disease with very poor prognosis and arises from the epithelial cells lining the esophagus. The incidence of Barrett's esophagus and esophageal adenocarcinoma is increasing rapidly and developing novel preventive and treatment strategies is of pivotal importance.

The terms “Barrett's disease” and “Barrett's esophagus” are used interchangeably herein.

Barrett's esophagus does not have any specific symptoms, although patients with Barrett's esophagus may have symptoms related to gastroesophageal reflux disease. It does, though, increase the risk of developing esophageal adenocarcinoma, which is a serious, potentially fatal cancer of the esophagus. Diagnosis of Barrett's esophagus may be done using endoscopy, histology, and/or using biomarkers, for instance as described in US 20120009597 A1.

Treatment of Barrett's esophagus with no malignant features, aim on reducing inflammation and epithelial-mesenchymal transition and includes treatment with compounds to relieve reflux or anti-reflux surgery. In case of malignant degeneration treatment is by endoscopic ablative therapies or surgically removing the affected part of the esophagus. Ablative therapy or surgery might be accompanied by neoadjuvant therapy and/or by postoperative chemotherapy. As stated herein, chemotherapy is highly toxic to the patient due to its parenteral systemic delivery. The remains therefore a need in the art for novel treatment options for esophageal cancer.

Esophageal squamous-cell carcinoma (ESCC) arises from the epithelial cells lining the esophagus. Regions of high incidence include Eastern to Central Asia, along the Rift Valley in East Africa, and into South Africa. There are many causes of ESCC, which vary among regions. Early studies in France associated smoking cigarettes and heavy alcohol consumption with high rates of ESCC, other risk factors for ESCC, include polycyclic aromatic hydrocarbons from a variety of sources, high-temperature foods, diet, and oral health. Esophageal squamous-cell carcinoma is diagnosed by endoscopic biopsy. ESCC is the eighth-most common cancer globally and has poor prognosis as diagnosis is often late. Thus, the development of novel preventive and treatment strategies is of pivotal importance. Further, esophageal squamous cell carcinoma is usually treated with neoadjuvant therapy followed by surgery or, alternatively, by definitive chemoradiotherapy involving a high toxicity for the patient. There remains therefore a need in the art for novel treatment options.

The drug delivery system described herein might be used for therapy, preferably for the treatment of esophageal diseases. Esophageal diseases to be treated according to the invention comprise but are not limited to an esophageal disease wherein preferably the esophageal disease is caused or related to a defect in the immune system, or preferably cancer.

Preferably, the esophageal disease to be treated in the context of the invention is Barrett's esophagus particularly Barrett' esophagus at different stages including metaplasia, low-grade and high-grade dysplasia. In another embodiment, the esophageal diseases to be treated in the context of the invention is preferably esophageal cancer, such as adenocarcinoma, esophageal junction carcinoma, or squamous cell carcinoma including but not limited to refractory cancer. In one embodiment, the treatment of esophageal cancer comprises a neoadjuvant therapy, i.e., chemotherapy followed by surgery. In one embodiment, the treatment of esophageal cancer comprises treatment of esophageal unresectable cancer, for example by chemotherapy as stand-alone therapy without subsequent surgery or ablative therapy. In one embodiment, the treatment of esophageal cancer comprises the treatment of esophageal cancer by postoperative chemotherapy, i.e., chemotherapy after surgery. In another embodiment, the esophageal diseases to be treated in the context of the invention is preferably esophageal stricture.

In a preferred embodiment of the invention, the drug delivery system according to the invention is adapted for the treatment of an esophageal disease such as Barrett' esophagus, particularly Barrett' esophagus at different stages including metaplasia, low-grade and high-grade dysplasia. In yet a further preferred embodiment of the invention, the drug delivery system according to the invention is adapted for the treatment of esophageal cancer, such as adenocarcinoma, esophageal junction carcinoma, or squamous-cell carcinoma, including but not limited to refractory cancer.

In one embodiment the drug delivery system according to the invention is adapted for the treatment of esophageal cancer by neoadjuvant therapy, i.e., chemotherapy followed by surgery. In one embodiment the drug delivery system according to the invention is adapted for the treatment of esophageal unresectable cancer, for example by chemotherapy as stand-alone therapy without subsequent surgery or ablative therapy. In one embodiment the drug delivery system according to the invention is adapted for the treatment of esophageal cancer by postoperative chemotherapy, i.e., chemotherapy after surgery. In another embodiment, the drug delivery system according to the invention is adapted for the treatment of esophageal stricture.

The administration frequency of the drug delivery device and the treatment period or the time point of administration is not limited and may be dependent of the specific disease to be treated and/or the amount of the active pharmaceutical ingredient per drug delivery device. For example, the drug delivery device can be administered once per day or twice day. If the drug delivery device is administered once a day, it is preferably administered in the evening to increase the patient's compliance. The drug delivery system of the invention is preferably administered before bedtime, i.e., after dinner and after oral hygiene. The treatment period may be between 7 days to 40 days, preferably 14 days to 30 days, more preferably from 20 days to 28 days. The treatment may comprise a single treatment cycle of the treatment period or multiple cycles, e.g., 2, 3, 4, 5, 6, 7, 8, 9, 10 or more treatment periods.

### Diagnosis

The drug delivery system described herein might also be used for diagnosis. Diagnosis might comprise monitoring the uptake of the agent into the cells or the route of the agent into tissue, organoids, cell culture or organs after administration. Diagnosis might also comprise monitoring of the treatment success such tumor size or the period between release of the agent from the drug delivery and cellular, tissue or organ uptake. For example, the preparation comprising the active pharmaceutical ingredient in combination with the diagnostic marker might be administered to a cell, tissue, organ, cell culture or organ. The route of the active pharmaceutical ingredient might be followed by monitoring the diagnostic marker as described herein. For the purpose of the invention, diagnosis might comprise in vivo and/or in vitro diagnosis.

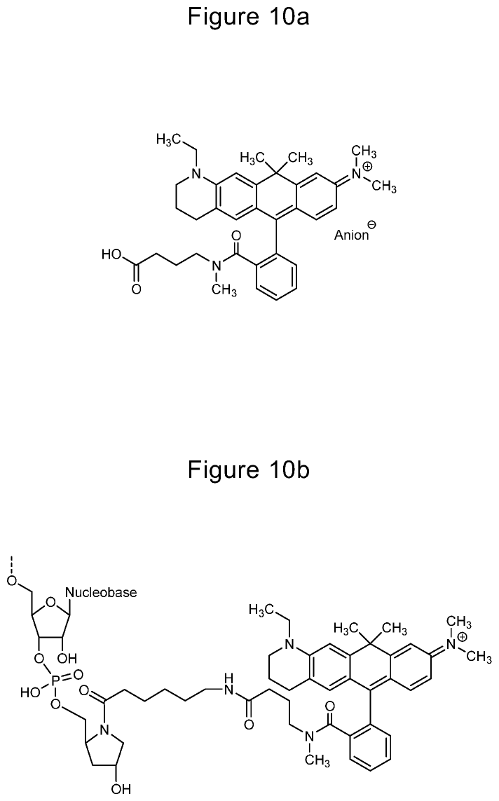

For the purpose of diagnosis, the agent effective in the treatment of an esophageal disease of the invention might be in combination with a diagnostic marker. Any diagnostic marker suitable for in vivo or in vitro diagnosis might be used. For example, the agent might be conjugated or otherwise associated, e.g., embedded, included, complexed, with a diagnostic marker. Alternatively, one or more atoms or functional groups of the agent might be replaced by the diagnostic marker. In principle, any known diagnostic marker might be used for diagnosis including but not limited to a radioisotope, a paramagnetic label such as gadolinium or iron oxide, a fluorophore, Near Infra-Red (NIR) fluorochrome or dye, an echogenic microbubble, an affinity label (for example biotin, avidin, etc.), enzymes, or any other suitable agent that may be detected by diagnostic imaging methods. In a specific, non-limiting example, the agent effective in the treatment or prevention of an esophageal disease, e.g., an antibody of the invention, may be conjugated to a near infrared fluorescence (NIRF) imaging dye, for example and not wishing to be limiting Cy5.5, Alexa680, Dylight680 or Dylight800, a radioisotope such as tritium (hydrogen-3), 11C (carbon), 13N (nitrogen), 15O (oxygen), 18F (fluorine), 32P (phosphorus), or 35S (sulfur). In another specific, non-limiting example, the agent effective in the treatment or prevention of an esophageal disease, e.g., an siRNA or antisense oligonucleotide, may be conjugated to fluorophore such as Atto633 (FIG. 10). In one embodiment, the inhibiting polynucleotide of the invention such as an siRNA, aptamer or an antisense oligonucleotide, are used in combination with a diagnostic marker. In one embodiment, the antibody of the invention is used in combination with a diagnostic marker. In one embodiment, the antiproliferative agent of the invention is used in combination with a diagnostic marker. Suitable imaging systems for diagnosis, particularly in vivo diagnosis, include but are not limited to PET scans, and SPECT scans involving radioisotopes.

### Active Pharmaceutical Ingredient

The term “active pharmaceutical ingredient” as used herein is used interchangeably with the term “active ingredient” or “API” and refers to an agent effective in the treatment or prevention of an esophageal disease.

By the term “therapeutically effective dose” or “effective amount” is meant a dose or amount that produces the desired effect for which it is administered. The exact dose or amount will depend on the purpose of the treatment and will be ascertainable by one skilled in the art using known techniques. The term “therapeutically effective amount” is an amount that is effective to ameliorate (a symptom of) a disease. A therapeutically effective amount can be a “prophylactically effective amount” as prophylaxis can be considered therapy.

An “agent effective in the treatment or prevention of an esophageal disease” refers to a compound that reduces, decreases, prevents, blocks, or interferes with an esophageal disease or disorder. In a particularly preferred embodiment, the agent effective in the treatment of an esophageal disease is an agent whose therapeutic application is limited by its high systemic toxicity, or which has a low systemic bioavailability, e.g., due to a high first pass effect and/or a loss of activity by passaging the gastrointestinal tract. The present invention enables or increases the therapeutic use of such agents due to the local availability of the agent at the treatment side. The agent effective in the treatment or prevention of an esophageal disease refers to any type of compound including but not limited to a polynucleotide such as an inhibiting polynucleotide, e.g., an siRNA molecule, an antisense oligonucleotide, a micro RNA (miRNA), an antagomir, or an aptamer; a small molecule; or a polypeptide such as an antibody or a ligand. The term also refers to a salt or any other derivative of an agent effective in the treatment or prevention of an esophageal disease if appropriate.

“Sequence identity” or the language “a sequence which is identical” is used to evaluate the similarity of two sequences, e.g., polynucleotide or polypeptide sequences; it is determined by calculating the percent of residues that are the same when the two sequences are aligned for maximum correspondence between residue positions. Any known method may be used to calculate sequence identity; for example, computer software is available to calculate sequence identity. Without wishing to be limiting, sequence identity can be calculated by software such as BLAST-P or BLAST-N of the National Center for Biotechnology Information, or any other appropriate software that is known in the art. Importantly, evaluation of sequence similarity between a comparative sequence with a given sequence is determined over the whole length of the comparative sequence. For example, the sequence of an siRNA molecule is the comparative sequence and sequence identity to a given sequence is determined via the BLAST-N program by the default algorithm for aligning two or more sequences. In another example, the antibody sequence as shown in SEQ ID NO: 11 is the comparative sequence and sequence identity to a given sequence is determined via the BLAST-P program by the default algorithm for aligning two or more sequences.

In one aspect of the invention, the agent effective in the treatment or prevention of an esophageal disease comprises or is an inhibiting polynucleotide, preferably an inhibiting polynucleotide such as an siRNA molecule, miRNA, antagomir, an antisense oligonucleotide, and an aptamer, more preferably an siRNA molecule, an antisense oligonucleotide, or an aptamer. In one embodiment, the inhibiting polynucleotide has a length of 10 to 100 nucleotides, preferably of 15 to 50 nucleotides, more preferably of 19 to 25 nucleotides. The polynucleotide might be “deoxyribonucleic acid” (DNA) or “ribonucleic acid” (RNA) or derivatives or modified versions thereof. The polynucleotide might be double-stranded or single-stranded.

In a preferred embodiment, the agent effective in the treatment or prevention of an esophageal disease comprises, preferably consists of, or is an siRNA molecule.

The term “small interfering RNA” or “siRNA” as used herein refers to exogenously synthesized RNA duplexes. Any method for producing an siRNA known in the art can be used. Those methods are known to the skilled person. siRNA comprise or consist of two RNA strands, an antisense (or guide) strand and a sense (or passenger) strand. These molecules display generally between 15-35 base pairs, preferably 19 to 25 base pairs, e.g., 15, 16, 17, 18, 19, 20, 21, 22, 23, 24, or 25 base pairs and are usually prepared intracellularly by enzymatical cleavage of larger RNA duplexes. The siRNA might contain varying degrees of sequence complementarity to their target mRNA or RNA transcript in the antisense strand. Preferably, the siRNA molecule of the present invention has sufficient sequence identity and/or sequence complementary to the target RNA transcript, preferably under physiological conditions thereby interfering, reducing, decreasing, inhibiting and/or blocking the expression and/or function of the polypeptide encoded by the target RNA transcript. Some, but not all, siRNA molecules include structures with overhangs. Overhangs have been described to be advantageous and may be present on the 5′ ends or on the 3′ ends of either strand as they reduce recognition by RNAses. Some siRNA molecules have an overhang on both 3′ ends of the strands, whereas others have an overhang on one strand only. Other siRNA molecules might be blunt-ended structures. Without being bound to any theory, these overhangs are said to further enhance resistance to nuclease (RNase) degradation. As used herein, the term “overhang” or “tail” refers to 2, 3, 4, 5, 6, 7, 8, 9, 10, 11, 12, 13, 14, 15, 16, 17, 18, 19, 20 or more sequential nucleotides at the 3′ end of one or both of the sense strand and the antisense strand that are not base-paired. The term siRNA refers to duplexes of two separate strands as well as to duplexes of a single strand which might be formed by self-complementary sequence portions such as hairpin structures to form a duplex region.

The term “duplex region” as used herein refers to the region in two complementary or partially complementary polynucleotides (e.g., a sense strand or an antisense strand) that form base pairs with one another.

The term “complementary” or “sequence complementary” refers to the hybridization or base-pairing between nucleotides. Hybridization preferably refers to stringent hybridization conditions such as washing for 1 h in 1×SSC and 0.1% SDS at 45° C., preferably at 48° C. and more preferably at 50° C., particularly for 1 h in 0.2×SSC and 0.1% SDS. Base-pairing might involve any base-pairing including but not limited to Watson/Crick base-pairing.

The terms “RNA transcript” or “mRNA transcripts”, include, but are not limited to primary transcripts, pre-mRNA transcript(s), transcript processing intermediates, mature mRNA(s) ready for translation, transcripts of a gene or genes, or nucleic acids derived from the mRNA transcript(s). Processing of pre-mRNA transcripts, along with the possible use of alternative promoters and alternative polyadenylation sites, allows a single gene to generate many different mature RNAs, by varying the pattern of splicing in a process known as alternative splicing. Alternative splicing can also introduce or remove regulatory elements to affect mRNA translation, localization or stability. These alternatively spliced mRNAs are translated into alternative splice form proteins that contain different amino acid sequences than the corresponding wildtype or canonical protein produced by normally spliced mRNA.

In one embodiment, the inhibiting polynucleotide comprises an siRNA molecule that targets an RNA transcript or a portion thereof encoding polypeptide which is involved in the development of an esophageal disease or disorder; or encoding a polypeptide which is expressed, or its expression is increased as a result of the disease or disorder. The siRNA molecule has sufficient sequence complementary and/or sequence identity to the target RNA transcript to inhibit, block, reduce or decrease the expression and/or function of the polypeptide encoded by the RNA transcript. In one embodiment, the polypeptide comprises or is a BMP2 or a BMP4 polypeptide, preferably as depicted in SEQ ID NO: 15 or SEQ ID NO: 16.

In one embodiment, the siRNA molecule comprises a duplex region, wherein the duplex region comprises a sense strand and an antisense strand wherein the sense strand and the antisense strand together form the duplex region, and the antisense strand is complementary to a target RNA transcript encoding BMP2 or BMP4 polypeptide. The complementary is sufficient to inhibit, block, reduce or decrease the expression and/or function of BMP2 and/or BMP4. In one embodiment, the siRNA molecule has sufficient complementary to the RNA transcript encoding BMP2 and/or BMP4 to inhibit, block, reduce or decrease the function of BMP2 and/or BMP4, e.g., BMP2 and/or BMP4 signaling, thereby restoring, or providing normal tissue lining of the esophagus or enhancing the formation of normal tissue lining of the esophagus and preventing or treating esophageal carcinoma. In a preferred embodiment the RNA transcript is as shown in SEQ ID NO: 20 or SEQ ID NO: 21. Preferably, the siRNA molecule comprises a duplex region comprising a sequence as shown in SEQ ID NO: 17, SEQ ID NO: 18 or SEQ ID NO: 19, preferably SEQ ID NO: 18, or any other sequence comprising a sequence identity of 60%, 70%, 75%, 80%, 85%, 90%, 95%, 96%, 97%, 98%, 99% or more between the siRNA molecule and the target RNA transcript or the portion thereof. The duplex region might have a length of 15 to 35 base pairs, preferably 17 to 30 base pairs, more preferably of 19 to 25 base pairs.

In one embodiment, the inhibiting polynucleotide comprises an siRNA molecule that targets an RNA transcript as shown in SEQ ID NO: 20 or SEQ ID NO: 21 or a portion thereof. In one embodiment, the siRNA molecule comprises or consists of a duplex region, wherein the duplex region comprises a sense strand and an antisense strand wherein the sense strand and the antisense strand together form the duplex region, and the antisense strand is complementary to the target RNA transcript as shown in SEQ ID NO: 20 or SEQ ID NO: 21 or a portion thereof.

In one embodiment, the siRNA molecule displays no overhang, i.e., it has blunt ends. In another embodiment, the siRNA molecule displays at least one overhang. The overhangs are thought to play a structural role for presenting the duplex to RISC. Each overhang might have one or more, preferably two, up to six sequential nucleotides. The nucleotide(s) in the overhang might comprise a nucleoside with a nucleobase such as adenine, cytosine, guanine, thymine or uracil. Preferably, the overhang comprises or consists of two sequential nucleotides, preferably deoxyribonucleotides, such as deoxyribonucleotides comprising the nucleoside deoxythymidine (dTdT). It is believed that the deoxyribonucleotides in the overhang confer nuclease resistance. It is also possible to use ribonucleotides in the overhang. The ribonucleotides might be complementary to the target transcript or might comprise the nucleoside uridine, preferably UU.

In a preferred embodiment, the siRNA molecule consists of a duplex region comprising a sequence as shown in SEQ ID NO: 17, SEQ ID NO: 18 or SEQ ID NO: 19, i.e., it has blunt-ends. In another preferred embodiment, the siRNA molecule essentially consists of a duplex region comprising a sequence as shown in SEQ ID NO: 17, SEQ ID NO: 18 or SEQ ID NO: 19, i.e., it consists of the duplex region and of one or more sequential overhang nucleotides preferably two, up to six sequential nucleotides as described herein.

In a preferred embodiment the siRNA molecule comprises, preferably consists of BMP2-siRNA 1, BMP2-siRNA 2 or BMP2-siRNA 3 as shown in the following table:

TABLE 1

| SEQ | | | siRNA |
|---|---|---|---|
| ID | | | desig- |
| NO: | Sequence 5′ -> 3′ | Strand | nation |
| 17 | GCAGGUCUUUGCACCAAGA | Sense | BMP2- |
| | | | siRNA 1 |
| 22 | UCUUGGUGCAAAGACCUGC | Antisense | |
| 18 | GCAACAGCCAACUCGAAAU | Sense | BMP2- |
| | | | siRNA 2 |
| 23 | AUUUCGAGUUGGCUGUUGC | Antisense | |
| 19 | GCUGUACCUUGACGAGAAU | Sense | BMP2- |
| | | | siRNA 3 |
| 24 | AUUCUCGUCAAGGUACAGC | Antisense | |

wherein each of the above sequences in Table 1 comprises an overhang of two nucleotides dTdT (deoxythymidine) or UU (uridine) attached to the 3′ end of each strand.

The siRNA molecule of the present invention might comprise chemically modified nucleotides or modified nucleotides. These modified nucleotides refer to non-standard nucleotides including non-naturally occurring deoxyribonucleotides or ribonucleotides. Such modifications are introduced to increase or improve the resistance to nucleases, the intracellular uptake, the target cell specificity, and/or the stability. Of course, such modifications are introduced without affecting the original inhibitory, blocking, reducing, or decreasing activity of the siRNA molecule on the target RNA transcript. The modifications might be introduced into the backbone of the polynucleotide, the sugar unit, and/or the nucleobase unit.

For example, the modification might be present in the backbone of the polynucleotide. For example, the phosphodiester linkage of the sense strand or the antisense strand of the siRNA molecule might be completely or partially replaced by a phosphorothioate or boron phosphate linkage to increase the stability of the siRNA molecule.

Another modification might be present in the sugar unit of the ribonucleotide of the siRNA molecule. For example, the ribose unit of the nucleoside may comprise one or more bridged nucleic acids to increase rigidity of the sugar unit thereby increasing the binding affinity and stability of the siRNA. Examples are LNA (locked nucleic acid) or ENA (ethylene-bridged nucleic acid). Such modification might be introduced in the ribose units of all nucleotides or of some nucleotides, preferably in nucleotides at the 3′ end or the 5′ end or both ends of the strands of the siRNA molecule.

For yet another example, a 2′—OH group of ribose unit may be substituted with —NH2, —NHR, —NR2, —COOR, —OR, —H, —F, —Cl, —Br, —I, —SH, —SR, —O-Me (or CH3, methyl group),-2′O-MOE (methoxyethyl) wherein R is substituted or unsubstituted C₁-C₆ alkyl, alkenyl, alkynyl, aryl, etc. as described in U.S. Pat. No. 9,080,171 and U.S. Pat.

Application No. 2019/0024082, all of which are incorporated herein by reference. For example, 2′—OH group of ribose unit of the 1^{st} and 2^{nd} nucleotides of the sense strand may be substituted with 2′-O-Me (methyl) or 2′—OH groups of ribose unit of 2nd nucleotide of the antisense strand may be substituted with 2′-O-Me, or 2′-OH of ribose unit of guanine (G) or uridine (U) containing nucleotides may be substituted with 2′-O-Me or 2′-F.

In yet another aspect of the invention, the agent effective in the treatment or prevention of an esophageal disease is an antisense oligonucleotide. An antisense oligonucleotide comprises a polymer of deoxyribonucleotides or ribonucleotides. Usually, the antisense oligonucleotide is a single stranded molecule and might contain varying degrees of complementarity to its target mRNA or RNA transcript. The antisense oligonucleotide has sufficient sequence complementary to the target RNA transcript to inhibit, block, reduce or decrease the expression and/or function of the polypeptide encoded by the RNA transcript. These molecules generally display a length of between 15-35 nucleotides, preferably 19 to 25 nucleotides, e.g., 15, 16, 17, 18, 19, 20, 21, 22, 23, 24, or 25 nucleotides and are usually synthesized exogenously. The antisense oligonucleotide of the present invention might comprise chemically modified nucleotides or modified nucleotides to increase binding affinity and/or stability of the antisense oligonucleotide. The modifications might be present in the backbone of the polynucleotide, the sugar unit or the nucleobase unit are disclosed herein in the context of siRNA molecules. In one embodiment, the antisense oligonucleotide is a morpholino. Any method for producing an antisense oligonucleotide known in the art can be used including but not limited to exogenous chemical synthesis. Those methods are known to the skilled person. The term antisense oligonucleotides might also intracellularly present oligonucleotides.

In one embodiment, the antisense oligonucleotide targets an RNA transcript or a portion thereof encoding a BMP2 or a BMP4 polypeptide, preferably as depicted in SEQ ID NO: 15 or SEQ ID NO: 16. In yet another embodiment, the antisense oligonucleotide comprises or consists of an antisense strand wherein the antisense strand is complementary to the target RNA transcript as shown in SEQ ID NO: 20 or SEQ ID NO: 21 or a portion thereof. The complementarity between the antisense strand and the target RNA transcript is sufficient to inhibit, block, reduce or decrease the expression and/or function of BMP2 and/or BMP4. In one embodiment, the antisense strand has sufficient complementary to the RNA transcript encoding BMP2 and/or BMP4 polypeptide to inhibit, block, reduce or decrease the function of BMP2 and/or BMP4, e.g., BMP2 and/or BMP4 signaling, thereby restoring, or providing normal tissue lining of the esophagus or enhancing the formation of normal tissue lining of the esophagus and preventing or treating esophageal carcinoma.

In one embodiment, the antisense oligonucleotide comprises a sequence as shown in SEQ ID NO: 22, SEQ ID NO: 23 or SEQ ID NO: 24, preferably SEQ ID NO: 23, or any other sequence comprising a sequence identity of 60%, 70%, 75%, 80%, 85%, 90%, 95%, 96%, 97%, 98%, 99% or more to between the complementary sequence of the antisense oligonucleotide molecule and the target RNA transcript. The duplex between the antisense oligonucleotide and the target transcript might have a length of 15 to 35 base pairs, preferably 17 to 30 base pairs, preferably of 19 to 25 base pairs.

In another aspect of the invention, the agent effective in the treatment or prevention of an esophageal disease is an aptamer. An aptamer is a single-stranded polynucleotide usually comprising one or more duplex regions formed by self-complementary sequence portions such as hairpins. The aptamer specifically binds to the target polypeptide by a specific three-dimensional structure. In one embodiment, the binding of the aptamer interferes with the activity of a BMP2 or a BMP4 polypeptide, preferably BMP2 or BMP4 as depicted in SEQ ID NO: 15 or SEQ ID NO: 16. Interfering refers to inhibiting, blocking, reducing, or decreasing the activity of BMP2 and/or BMP4 such as BMP2 and/or BMP4 signaling. Preferably, the aptamer interferes with the activity of BMP2 and/or BMP4 by binding to BMP2 and/or BMP4 to the active site of BMP2 and/or BMP4, e.g., the receptor binding site for binding the receptor on the target cell. In one embodiment, the binding of the aptamer to BMP2 and/or BMP4 polypeptide inhibits, blocks, reduces or decreases the function of BMP2 and/or BMP4, e.g., BMP2 and/or BMP4 signaling, thereby restoring, or providing normal tissue lining of the esophagus or enhancing the formation of normal tissue lining of the esophagus and preventing or treating esophageal carcinoma. The binding usually occurs through non-covalent binding, e.g., electrostatic interactions, stacking of flat moieties, shape complementation and/or hydrogen bonding. Any method for producing an aptamer known in the art can be used. Those methods are known to the skilled person including but not be limited to SELEX (systematic evolution of ligands by exponential enrichment).

The inhibiting polynucleotides such as siRNA molecules or antisense oligonucleotides act intracellularly. Thus, inhibiting polynucleotides administered with the drug delivery system of the invention are advantageously introduced into the cells of the esophagus. For the purpose of the invention, it is useful to administer the inhibiting polynucleotides in combination with a nucleic acid delivery system. The nucleic acid delivery system may increase or enable the intracellular delivery of the inhibiting polynucleotide. The inhibiting polynucleotides might be in a complex, linked, embedded, attached, or enclosed with or in the nucleic acid delivery system. For the purpose of the invention, the nucleic acid delivery system includes but is not limited to a liposome, a lipid bilayer, a cationic polymer, micelle, emulsion or a nanoparticle such as a lipid nanoparticle or a polymer nanoparticle. The cationic polymer for delivering nucleic acid may include natural polymer such as chitosan, atelocollagen, cationic polypeptide, and the like and synthetic polymer such as poly(L-lysine), linear or branched polyethylene imine (PEI), cyclodextrin-based polycation, dendrimer, and the like.

The inhibiting polynucleotides of the invention might also comprise a diagnostic marker. Such diagnostic marker might be attached at, e.g., conjugated to, any site of the polynucleotide, preferably to the 3′ and/or 5′ end of the polynucleotides. Diagnostic markers including inhibiting polynucleotides such as antisense oligonucleotides or siRNA molecules might be used for diagnosis as described herein.

In another aspect of the invention, the agent effective in the treatment or prevention of an esophageal disease is an antibody or a binding fragment thereof.

In one embodiment, the antibody or a binding fragment thereof targets a polypeptide or a portion thereof which is involved in the development of an esophageal disease or disorder, or a polypeptide which is expressed, or its expression is increased, as a result of the disease or disorder.

In one embodiment, the target polypeptide encodes a BMP2 or a BMP4 polypeptide, preferably as depicted in SEQ ID NO: 15 or SEQ ID NO: 16. In another embodiment, the target polypeptide encodes PD-1. In a further embodiment, the target polypeptide encodes and EGF-receptor such as erb-b2 receptor tyrosine kinase 2.

As used herein, the term “BMP2” is used to refer to mature bone morphogenic protein 2, preferably of human origin. The nucleotide sequence of human pro-BMP2 is publicly available by reference to GenBank Accession No. NM_001200. A portion of the amino acid sequence of mature BMP2 is presented herein as SEQ ID NO: 14. The numbering of the residues as used herein refer to the positions of SEQ ID NO: 14.

As used herein, the term “BMP4” is used to refer to human mature bone morphogenic protein 4. The nucleotide sequence of human pro-BMP4 is publicly available by reference to GenBank Accession No. NM_130851. A portion of the amino acid sequence of mature BMP4 is presented herein as SEQ ID NO: 1. The numbering of the residues as used herein refer to the positions of SEQ ID NO:1.

The term “BMP4 signaling” as used herein refers to the ability of BMP4 to activate the canonical (the phosphorylation of SMAD 1/5/8) Assays to test BMP4 signaling are described for instance in Shaifur Rahman et al., “TGF-ß/BMP signaling and other molecular events: regulation of osteoblast genesis and bone formation” in Bone Research 3, Article number: 15005 (2015).

The term “BMP2 signaling” as used herein refers to the ability of BMP2 to activate the canonical (the phosphorylation of SMAD 1/5/8).

The term the “wrist” of BMP4 as used herein refers to the region within the BMP4 protein which binds to type I receptors, such as BMPR1a and BMPR1b.

The term “knuckle” of BMP4 as used herein refers to the region within the BMP4 protein which binds to type II receptors, such as BMPR2, ActRIl and ActRIIB.

In a preferred embodiment said epitope is located in the wrist within residues 10-17, 24-31, 45-72, 89, 91, 101, 103, 104, and 106 of BMP4 (SEQ ID NO: 1).

The term “binds within” a certain epitope as used herein refers to an Ig-like molecule which binds to one, but preferably at least 2, 3, 5, 6, 7, 8 or more residues within an epitope of BMP4. In preferred embodiments, said Ig-like molecule does not (substantially) bind to any other epitope of BMP4.

The term “binds to” as used herein in the context of the interaction between an Ig-like molecule and an epitope, is intended to refer to the capability of the Ig-like molecule of binding to an antigen by an immunoglobulin variable region of the Ig-like molecule with a dissociation constant of KD of 1×10⁻⁶ M or less, preferably of 1×10⁻⁷ M or less, more preferably 1×10⁻⁸ M or less, more preferably 6×10⁻⁹ M or less, more preferably 3×10⁻⁹ M or less, more preferably 2×10⁻⁹ M or less.

As used herein, an Ig-like molecule that “specifically binds” to a certain epitope is intended to refer to an Ig-like molecule that binds to said certain epitope, but preferably does not (substantially) bind to another epitope.

The term “does not substantially bind” to an epitope, as used herein, means does not bind or does not bind with a high affinity to the epitope, i.e., binds to the epitope with a KD of 1×10⁻⁶ M or more, more preferably 1×10⁻⁵ M or more, more preferably 1×10⁻⁴ M or more, more preferably 1×10⁻³ M or more, even more preferably 1×10² M or more.

The term “antigen-binding portion” of an antibody (or “antigen-binding fragment thereof” or “antigen-binding fragment” or “binding fragment” or “binding fragment thereof” or “antibody fragment”), as used herein, refers to one or more fragments of an antibody that retain the ability to specifically bind to an antigen (e.g., to an epitope within the BMP4 or PD-1 protein). Typically, an “binding fragment” or an “antibody fragment” retains at least 10% of the parental binding activity when that activity is expressed on a molar basis. Preferably, an antibody fragment retains at least 20%, 50%, 70%, 80%, 90%, 95% or 100% or more of the parental antibody's binding affinity for the target. It has been shown that the antigen-binding function of an Ig-like molecule can be performed by fragments of a full-length antibody. The antibody fragment may be obtained by manipulation of a naturally occurring antibody or may be obtained using recombinant methods. Examples of binding fragments encompassed within the term “antigen-binding portion” of an antibody include (i) a Fab fragment, a monovalent fragment consisting of the VL, VH, CL and CHI domains; (ii) a F(ab′)2 fragment, a bivalent fragment comprising two Fab fragments linked by a disulfide bridge at the hinge region; (iii) a Fab′ fragment, which is essentially an Fab with part of the hinge region {see, FUNDAMENTAL IMMUNOLOGY (Paul ed, 3.sup.rd ed. 1993]; (iv) a Fd fragment consisting of the VH and CHI domains; (v) a Fv fragment consisting of the VL and VH domains of a single arm of an antibody, (vi) a dAb fragment (Ward et al., (1989) Nature 341:544-546), which consists of a VH domain; (vii) an isolated complementarity determining region (CDR); and (viii) a nanobody, a heavy chain variable region containing a single variable domain and two constant domains. Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al. (1988) Science 242:423-426; and Huston et al. (1988), Proc. Natl. Acad. ScL USA 85:5879-5883). Such single chain antibodies are also intended to be encompassed within the term “antigen-binding portion” of an antibody. These antibody fragments are obtained using conventional techniques known to those with skill in the art, and the fragments are screened for utility in the same manner as are intact antibodies. In a non-limiting example, the antibody fragment may be a single domain antibody (single-domain antibody) derived from naturally occurring sources. The term single domain antibody may also be a multivalent presentation thereof. Heavy chain antibodies of camelid origin lack light chains and thus their antigen binding sites consist of one domain, termed VHH. Single-domain antibodies have also been observed in shark and are termed VNARs; other single-domain antibodies may be engineered based on human heavy or light chain sequences. As used herein, “single-domain antibody” includes those directly isolated from VL, VH, VHH or VNAR reservoir of any origin through phage display or other display technologies and those generated through further modification of such single-domain antibody by humanization, affinity maturation, stabilization, solubilization (e.g., camelization), or other methods of antibody engineering. Also encompassed by the present invention are homologues, derivatives, or fragments that retain or improve the antigen-binding function and specificity of the single-domain antibody. A person of skill in the art would be well-acquainted with the structure of a single-domain antibody. A single-domain antibody comprises a single immunoglobulin domain that retains the immunoglobulin fold; most notably, only three CDR form the antigen-binding site. However, not all CDR may be required for binding the antigen. For example, and without wishing to be limiting, one, two, or three of the CDR may contribute to binding and recognition of the antigen by the single-domain antibody of the present invention. The CDR of the single-domain antibody are referred to herein as CDR1, CDR2, and CDR3, and are based on Kabat numbering (Kabat et al. 1991). The single-domain antibody may be of camelid origin, and thus may be based on camelid framework regions; alternatively, the CDR may be grafted onto the framework regions of other antibody domains, for example but not limited to VNAR, human VH or human VL framework regions. In yet another alternative, the CDR described above may be grafted onto the framework regions of other types of antibody fragments (Fv, scFv, Fab). Also encompassed by the present invention are diabodies. A “diabody” is a small antibody fragment with two antigen-binding sites. The fragments comprises a heavy chain variable domain (VH) connected to a light chain variable domain (VL) in the same polypeptide chain (VH-VL or VL-VH). By using a linker that is too short to allow pairing between the two domains on the same chain, the domains are forced to pair with the complementary domains of another chain and create two antigen-binding sites. Diabodies are described more fully in, e.g., EP 404,097; WO 93/11161; and Holliger et al. (1993) Proc. Natl. Acad. Sci. USA 90: 6444-6448.

Preferred antibodies of the present invention are disclosed in WO 2016/043577 and WO 2018/193129 which are incorporated herein in full.

Preferably, an antibody or a binding fragment thereof according to the invention binds within, preferably binds to an epitope consisting of, residues 10-17, 45-56, and 69 of BMP4 (SEQ ID NO:1). This epitope contains a hydrophobic groove, which is believed to be important for BMP4 specific binding. An advantage of the antibodies or binding fragments thereof according to this embodiment is that said antibodies or binding fragments thereof have a low affinity for other members of the BMP family and are highly effective in specifically inhibiting BMP4 signaling. Preferably, said antibody or a binding fragment thereof does not substantially bind to BMP2, BMP5, BMP6 or BMP7. A further advantage thereof is that said antibody, or a binding fragment thereof does not inhibit BMP2 mediated signaling, thereby diminishing or even avoiding adverse side effects when used in vivo. More preferably, said antibody or a binding fragment thereof specifically binds to at least one residue selected from the group consisting of Lys10, Asn11, Lys12, Asn13, Cys14, Arg15, Arg16, and His17, at least one residue selected from the group consisting of Gly45, Asp46, Cys47, Pro48, Phe49, Pro50, Leu51, Ala52, Asp53, His54, Leu55 and Asn56, and to Ser69 of BMP4. Preferably, said antibody or a binding fragment thereof binds to more than 4, 5, 6, 7, 8, 9, 10, 11, 12, 13, 14, 15 preferably 16 residues thereof. In a highly preferred embodiment, said antibody or a binding fragment thereof specifically binds to at least Lys12, Arg15, Asp46, and Pro50 of BMP4.

In a preferred embodiment, an antibody or a binding fragment thereof according to the invention is a single chain antibody. In a preferred embodiment, said antibody or a binding fragment thereof comprises a heavy chain CDR2 consisting of the amino acid sequence of SEQ ID NO: 3 or a sequence not differing more than 2 amino acid thereof, a heavy chain CDR3 consisting of the amino acid sequence of SEQ ID NO:4, or a sequence not differing more than 1 amino acid thereof, and preferably further a heavy chain CDR1 consisting of the amino acid sequence of SEQ ID NO: 2 or a sequence not differing more than 1 amino acid thereof. In a highly preferred embodiment, said antibody or a binding fragment thereof comprises the amino acid sequence of SEQ ID NO: 11.

In another embodiment, an antibody or a binding fragment thereof according to the invention binds within, preferably binds to an epitope consisting of, residues 24-31, 57-68, 70-72, 89, 91, 101, 103, 104 and 106 of BMP4 (SEQ ID NO:1). This region represents a “hydrophobic pocket” within the wrist epitope of BMP4. An advantage of antibodies or binding fragments thereof which bind to this region is that these antibodies or binding fragments thereof have a very high affinity for BMP4 and BMP2 and are also capable of efficiently inhibiting BMP4 and BMP2 signaling. Preferably, said antibody or a binding fragment thereof specifically binds to at least one residue selected from the group consisting of Ser24, Asp25, Val26, Gly27, Trp28, Asn29, Asp30, Trp31; at least one residue selected from the group consisting of Ser57, Thr58, Asn59, His60, Ala61, Ile62, Val63, Gln64, Thr65, Leu66, Val67, and Asn68; at least one residue selected from the group consisting of Val70, Asn71 and Ser72; at least one residue selected from the group consisting of Tyr103 and Gln104; and Met89, Tyr91, Lys101, and to Met106 of BMP4. Preferably, said antibody or a binding fragment thereof binds to more than 9, 10, 11, 12, 13 preferably 14 residues thereof. In a highly preferred embodiment, said antibody or a binding fragment thereof specifically binds to Asp30, Trp31, Leu66 and Lys101 of BMP4.

In a preferred embodiment, said antibody or a binding fragment thereof is a single chain antibody. In a preferred embodiment, said single chain antibody or a binding fragment thereof is capable of binding to a “hydrophobic pocket” region within the wrist epitope of BMP4 as described above. In a preferred embodiment, an antibody or a binding fragment thereof according to the invention comprises a heavy chain CDR3 consisting of the amino acid sequence of SEQ ID NO: 7 or a sequence not differing more than 1 amino acid thereof. Without wishing to be bound by theory, it is believed that this CDR3 is important for the binding interaction with the hydrophobic pocket of the wrist of BMP4. Preferably, said antibody or a binding fragment thereof further comprises a heavy chain CDR1 consisting of the amino acid sequence of SEQ ID NO: 5 or a sequence not differing more than 1 amino acid thereof, and a heavy chain CDR2 consisting of the amino acid sequence of SEQ ID NO:6, or a sequence not differing more than 1 amino acid thereof. In a highly preferred embodiment, said antibody or a binding fragment thereof comprises the amino acid sequence of SEQ ID NO: 12.

In yet another embodiment an antibody or a binding fragment thereof of the invention binds within, preferably binds to an epitope consisting of, residues 34, 35, 39, 86-88, 90, 97, 98, 100, 102 and 109 of BMP4 (SEQ ID NO: 1). This region represents the so called “knuckle” epitope of BMP4. Antibodies or binding fragments thereof specifically binding to residues in this region also have a high affinity for BMP4, but in addition also a high affinity for BMP2 and slightly less for BMP5, and BMP6. Preferably, said antibody or a binding fragment thereof binds specifically to Ala34, Gln39, Ser88, Leu90 and Leu100 of BMP4.

In a preferred embodiment, said antibody or a binding fragment thereof is a single chain antibody. In a preferred embodiment, an antibody or a binding fragment thereof capable of binding to said knuckle as described above comprises a heavy chain CDR1 consisting of the amino acid sequence of SEQ ID NO: 8 or a sequence not differing more than 1 amino acid thereof and a heavy chain CDR2 consisting of the amino acid sequence of SEQ ID NO:9, or a sequence not differing more than 1 amino acid thereof. Without wishing to be bound by theory, it is believed that these CDRs are important for the binding interaction with the knuckle of BMP4. Said antibody or a binding fragment thereof preferably further comprises a heavy chain CDR3 consisting of the amino acid sequence of SEQ ID NO: 10 or a sequence not differing more than 1 amino acid thereof. In a preferred embodiment, said antibody or a binding fragment thereof comprises the amino acid sequence of SEQ ID NO: 13.

In a preferred embodiment, antibodies or binding fragments thereof, which bind to, preferably bind to an epitope consisting of, the hydrophobic pocket of the wrist of BMP4 are used. These antibodies or binding fragments thereof inhibit the signaling of both BMP2 and BMP4. Such antibodies or binding fragments thereof are disclosed in WO2016/042050 on p. 22-24. Preferably, said antibody specifically binds to at least one residue selected from the group consisting of Ser24, Asp25, Val26, Gly27, Trp28, Asn29, Asp30, Trp31; at least one residue selected from the group consisting of Ser57, Thr58, Asn59, His60, Ala61, Ile62, Val63, Gln64, Thr65, Leu66, Val67, and Asn68; at least one residue selected from the group consisting of Val70, Asn71 and Ser72; at least one residue selected from the group consisting of Tyr103 and Gln104; and Met89, Tyr91, Lys101, and to Met106 of BMP4 (SEQ ID NO: 1). Preferably, said antibody or a binding fragment thereof binds to more than 9, 10, 11, 12, 13 preferably 14 residues thereof. In a highly preferred embodiment, said antibody or a binding fragment thereof specifically binds to Asp30, Trp31, Leu66 and Lys101.

In a preferred embodiment, said antibody or a binding fragment thereof is a single chain antibody. In a preferred embodiment, an antibody or a binding fragment thereof according to the invention comprises a heavy chain CDR3 consisting of the amino acid sequence of SEQ ID NO: 7 or a sequence not differing more than 1 amino acid thereof. Without wishing to be bound by theory, it is believed that this CDR3 is important for the binding interaction with the hydrophobic pocket of the wrist of BMP4.

Preferably, said antibody or a binding fragment thereof further comprises a heavy chain CDR1 consisting of the amino acid sequence of SEQ ID NO: 5 or a sequence not differing more than 1 amino acid thereof, and a heavy chain CDR2 consisting of the amino acid sequence of SEQ ID NO:6, or a sequence not differing more than 1 amino acid thereof. In a highly preferred embodiment, said antibody or a binding fragment thereof comprises the amino acid sequence of SEQ ID NO: 12.

In a preferred embodiment, the invention provides a hetero or homo multimeric molecule with increased antigen affinity for the antigens and/or an increased inhibitory effect on BMP signaling. The invention therefore provides a multimeric antibody comprising at least one, more preferably at least two antibodies which bind to the hydrophobic pocket of the wrist of BMP4 as described above, or a multimeric antibody comprising at least one, more preferably at least two antibodies which bind the knuckle epitope of BMP4 as described above.

In another preferred embodiment, said antibodies or binding fragments thereof may have a substantially identical sequence to SEQ ID NO: 11, 12 or 13. A substantially identical sequence may comprise one or more conservative amino acid mutations. It is known in the art that one or more conservative amino acid mutations to a reference sequence may yield a mutant peptide with no substantial change in physiological, chemical, or functional properties compared to the reference sequence; in such a case, the reference and mutant sequences would be considered substantially identical polypeptides.

Conservative amino acid mutation as used herein may include addition, deletion, or substitution of an amino acid; in one non-limiting example, the conservative amino acid mutation is a conservative amino acid substitution. A conservative amino acid substitution is defined herein as the substitution of an amino acid residue for another amino acid residue with similar chemical properties (e.g., size, charge, or polarity).

A conservative amino acid substitution as used herein may substitute a basic, neutral, hydrophobic, or acidic amino acid for another of the same group. By the term “basic amino acid” it is meant hydrophilic amino acids having a side chain pK value of greater than 7, which are typically positively charged at physiological pH. Basic amino acids include histidine (His or H), arginine (Arg or R), and lysine (Lys or K). By the term “neutral amino acid” (also “polar amino acid”), it is meant hydrophilic amino acids having a side chain that is uncharged at physiological pH, but which has at least one bond in which the pair of electrons shared in common by two atoms is held more closely by one of the atoms. Polar amino acids include serine (Ser or S), threonine (Thr or T), cysteine (Cys or C), tyrosine (Tyr or Y), asparagine (Asn or N), and glutamine (Gln or Q). The term “hydrophobic amino acid” (also “non-polar amino acid”) is meant to include amino acids exhibiting a hydrophobicity of greater than zero according to the normalized consensus hydrophobicity scale of Eisenberg (1984).

Hydrophobic amino acids include proline (Pro or P), isoleucine (Ile or I), phenylalanine (Phe or F), valine (Val or V), leucine (Leu or L), tryptophan (Trp or W), methionine (Met or M), alanine (Ala or A), and glycine (Gly or G).

“Acidic amino acid” refers to hydrophilic amino acids having a side chain pK value of less than 7, which are typically negatively charged at physiological pH. Acidic amino acids include glutamate (Glu or E), and aspartate (Asp or D).

The substantially identical sequences of the present invention may be at least 70% identical; in another example, the substantially identical sequences may be at least 70, 71, 72, 73, 74, 75, 80, 85, 90, 91, 92, 93, 94, 95, 96, 97, 98, 99, or 100% identical at the amino acid level to sequences described herein. Importantly, the substantially identical sequences retain the activity and specificity of the reference sequence. As would be known to one of skill in the art, amino acid residues of an antibody or a binding fragment thereof, particularly within the framework regions may be mutated (substituted or deleted) without affecting the functional properties of the antibody or a binding fragment thereof (antigen recognition and binding).

Standard assays to evaluate the binding ability of the Ig-like molecules toward one or more epitopes are known in the art including, for example, ELISAs, Western blots, flow cytometry and RIAs. The binding kinetics (e.g., binding affinity) of the antibodies also can be assessed by standard assays known in the art, such as by ELISA, Scatchard and Biacore analysis. Preferably, said Ig-like molecule has an affinity KD lower than 1590 pM⁻¹ for BMP4, as determined using surface plasmon resonance analysis. Preferably, said KD is lower than 1000 pM⁻¹, more preferably lower than 1500, 1400, 1300, 1200, 1100, 1000, 900, 800, 750, 700, 650, 635, 600, 575, 513, 393, 100, 91, 75, 50, 32, 25, 10 pM⁻¹.

Furthermore, it is described that the Ig-like molecules of the invention inhibit BMP4 signaling to the same extent as Noggin. This may be explained by the wide reach of contact points of Noggin, which simultaneously masks both BMPRIa and BMPR2 epitopes. Whereas a C-terminal finger-like region of Noggin is the responsible for blocking BMPR2 binding, an N-terminal clip-like region binds to the BMPRIa epitope. This remarkable structural reach is conserved amongst BMP antagonists and might explain their lack of BMP-specificity. Assays for determining whether an Ig-like molecule competes with Noggin are known in the art. In a preferred embodiment, such assays include surface plasmon resonance (SPR) sandwich cross-binding or “epitope binning” assay, preferably as described in Examples 7 and 8 on page 48-52 of WO 2016/043577.

The binding epitopes of the antibodies can be determined by standard methods known in the art including but not limited to “epitope binning” experiments (surface plasmon resonance (SPR) sandwich cross-binding) and preferably validated by subsequent HADDOCK modelling, e.g., by using the HADDOCK software (High Ambiguity Driven protein-protein DOCKing) (Domininguez et al, 2003) as described in Examples 7, 8 and 9 on pages 48-55 of WO 2016/043577 which are incorporated by reference herein.

Specifically, in the “epitope binning” or surface plasmon resonance (SPR) sandwich cross-binding assay, a ligand is bound to control molecule which is immobilized on a support such as a chip. After binding of the ligand, the ligand bound to the control molecule is contacted with a second molecule. The second molecule will only bind to the bound ligand if its epitope on the ligand is still accessible. Thus, binding will not occur if the control molecule and the second molecule compete for binding to the ligand because they share the same epitope. For the purpose of the invention, the control molecule might be a first inventive antibody, the ligand might be an antigen such as BMP4 and the secondary molecule might be a second inventive antibody which is different from the first antibody.

Mutational experiments using mutated variants of the second molecule, such as antibodies, can be carried out to further validate the modelling or confirm the binding to certain epitopes. Mutated variants can be produced by any method known in the art including but limited to site directed mutagenesis by overlap extension PCR.

The antibodies of the invention might be produced by any method known in the art including but limited to the methods as described in WO 2016/043577, which are incorporated herein in full, particularly page 26, line 15 to page 33, line 28).

In another embodiment of an antibody or a fragment thereof which targets a polypeptide or a portion thereof which is involved in the development of an esophageal disease or disorder, or a polypeptide which is expressed, or its expression is increased, as a result of the disease or disorder, the antibody or fragment thereof targets a polypeptide which encodes PD-1, preferably human PD-1.

In some embodiments, the antibody or antibody fragment blocks binding of PD-L1 and/or PD-L2 to PD-1, preferably binding of human PD-L1 and/or human PD-2 to human PD-1.

The term “chimeric” antibody refers to antibodies in which a portion of the heavy and/or light chain is derived from a particular species or belonging to a particular antibody class or subclass, while the remainder of the chain(s) is derived from another species or belonging to another antibody class or subclass, as well as fragments of such antibodies, so long as they exhibit the desired biological activity (See, for example, U.S. Pat. No. 4,816,567 and Morrison et al., 1984, Proc. Natl. Acad. Sci. USA 81:6851-6855).

“Humanized” forms of non-human (for example, murine) antibodies are chimeric antibodies that contain minimal sequence derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, Fv framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin. For further details, see Jones et al., Nature 321:522-525 (1986); Riechmann et al., Nature 332:323-329 (1988); and Presta, Curr. Op. Struct. Biol. 2:593-596 (1992).

The term “hypervariable region,” as used herein, refers to the amino acid residues of an antibody which are responsible for antigen-binding. The hypervariable region comprises amino acid residues from a “complementarity determining region” or “CDR,” defined by sequence alignment, for example residues 24-34 (L1), 50-56 (L2) and 89-97 (L3) in the light chain variable domain and 31-35 (H1), 50-65 (H2) and 95-102 (H3) in the heavy chain variable domain (see Kabat et al., 1991, Sequences of proteins of Immunological Interest, 5th Ed. Public Health Service, National Institutes of Health, Bethesda, Md.) and/or those residues from a “hypervariable loop” (HVL), as defined structurally, for example, residues 26-32 (L1), 50-52 (L2) and 91-96 (L3) in the light chain variable domain and 26-32 (H1), 53-55 (H2) and 96-101 (H3) in the heavy chain variable domain (see Chothia and Leskl, 1987, J. Mol. Biol. 196:901-917). “Framework” or “FR” residues are those variable domain residues other than the hypervariable region residues as herein defined.

A “human antibody” is an antibody that possesses an amino acid sequence corresponding to that of an antibody produced by a human and/or has been made using any of the techniques for making human antibodies disclosed herein. This definition specifically excludes a humanized antibody that comprises non-human antigen-binding residues.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to conventional (polyclonal) antibody preparations that typically include different antibodies directed against different determinants (epitopes), each monoclonal antibody is directed against a single determinant on the antigen. The modifier “monoclonal” indicates the character of the antibody as being obtained from a substantially homogeneous population of antibodies and is not to be construed as requiring production of the antibody by any particular method. For example, the monoclonal antibodies to be used in accordance with the present invention may be made by the hybridoma method first described by Kohler et al., 1975, Nature 256:495, or may be made by recombinant DNA methods (see, for example, U.S. Pat. No. 4,816,567). The “monoclonal antibodies” may also be isolated from phage antibody libraries using the techniques described in Clackson et al., 1991, Nature 352:624-628 and Marks et al., 1991, J. Mol. Biol. 222:581-597, for example. The monoclonal antibodies herein specifically include “chimeric” antibodies.

Preferred antibodies of the present invention are disclosed in WO 2008/156712 which is incorporated herein in full, particularly pages 6 to 11.

In one embodiment, the PD-1 antibody or antibody fragment of the invention includes an antibody or antibody fragment which binds to PD-1, preferably human PD-1, comprising:
a. at least one CDR (complementary determining region) selected from the group consisting of SEQ ID NOs: 30, 31, 32, 36, 37 and 38, or a variant of any said sequence; and/or
b. at least one a CDR selected from the group consisting of SEQ ID NOs: 33, 34, 35, 39, 40 and 41, or a variant of any said sequence.

In one embodiment, the antibody or antibody fragment which binds to PD-1, preferably human PD-1, comprises:
a. light chain CDRs SEQ ID NOs: 30, 31 and 32, or variants of any said sequences; and/or heavy chain CDRs SEQ ID NOs: 33, 34 and 35, or variants of any said sequences; or
b. light chain CDRs SEQ ID NOs: 36, 37 and 38 or variants of any said sequences; and/or heavy chain CDRs SEQ ID NOs: 39, 40 and 41 or variants of any said sequences.

In one embodiment, the antibody or antibody fragment which binds to PD-1, preferably human PD-1, comprises:
a. a heavy chain variable region comprising an amino acid sequence selected from the group consisting of:
i. SEQ ID NO: 26 or a variant thereof;
ii. SEQ ID NO: 28 or a variant thereof;
iii. amino acid residues 20 to 139 of SEQ ID NO: 42 or a variant thereof; and
iv. an amino acid sequence having at least 50%, 60%, 70%, 75%, 80%, 85%, 90%, 95%, 99% sequence identity to amino acid residues 20 to 139 of SEQ ID NO: 42;
and further comprising
b. a light chain variable region comprising an amino acid sequence selected from the group consisting of:
i. SEQ ID NO: 27 or a variant thereof;
ii. SEQ ID NO: 29 or a variant thereof;
iii. amino acid residues 20 to 130 of SEQ ID NO: 44 or a variant thereof;
iv. amino acid residues 20 to 130 of SEQ ID NO: 45 or a variant thereof;
v. amino acid residues 20 to 130 of SEQ ID NO: 46 or a variant thereof; and
vi. an amino acid sequence having at least 50%, 60%, 70%, 75%, 80%, 85%, 90%, 95%, 99% sequence identity to amino acid residues 20 to 130 of SEQ ID NO: 44, 45 or 46.

Specifically, in one embodiment, the invention comprises an antibody or antigen binding fragment comprising a heavy chain variable region SEQ ID NO: 26 or a variant thereof; and/or a light chain variable region comprising SEQ ID NO: 27 or a variant thereof.

In one embodiment, the invention comprises an antibody or antigen binding fragment comprising a heavy chain variable region SEQ ID NO: 28 or a variant thereof and/or a light chain variable region comprising SEQ ID NO: 29 or a variant thereof.

In one embodiment, the invention comprises an antibody or antigen binding fragment comprising a heavy chain variable region comprising amino acid residues 20 to 139 of SEQ ID NO: 42 or a variant thereof; and/or a light chain variable region comprising amino acid residues 20 to 130 of SEQ ID NO: 44 or a variant thereof.

In one embodiment, the invention comprises an antibody or antigen binding fragment comprising a heavy chain variable region comprising amino acid residues 20 to 139 of SEQ ID NO: 42 or a variant thereof; and/or a light chain variable region comprising amino acid residues 20 to 130 of SEQ ID NO: 45 or a variant thereof.

In one embodiment, the invention comprises an antibody or antigen binding fragment comprising a heavy chain variable region comprising amino acid residues 20 to 139 of SEQ ID NO: 42 or a variant thereof; and/or a light chain variable region comprising amino acid residues 20 to 130 of SEQ ID NO: 46 or a variant thereof.

In one embodiment, the invention comprises an antibody or antigen binding fragment comprising a heavy chain variable region comprising an amino acid sequence having at least 50%, 60%, 70%, 75%, 80%, 85%, 90%, 95%, 99% sequence identity to amino acid residues 20 to 139 of SEQ ID NO: 42; and/or a light chain variable region comprising and an amino acid sequence having at least 50%, 60%, 70%, 75%, 80%, 85%, 90%, 95%, 99% sequence identity to amino acid residues 20 to 130 of SEQ ID NO: 44, 45 or 46.

In one embodiment, the antibody or antibody fragment which binds to PD-1, preferably human PD-1, comprises:
a. a heavy chain comprising an amino acid sequence selected from the group consisting of:
i. amino acid residues 20 to 466 of SEQ ID NO: 43 or a variant thereof, and
ii. amino acid residues 20 to 469 of SEQ ID NO: 47 or a variant thereof; and
b. a light chain comprising an amino acid sequence selected from the group consisting of: i. amino acid residues 20 to 237 of SEQ ID NO: 48 or a variant thereof;
ii. amino acid residues 20 to 237 of SEQ ID NO:49 or a variant thereof, and
iii. amino acid residues 20 to 237 of SEQ ID NO: 50 or a variant thereof.

In one embodiment, the invention provides an isolated antibody or antibody fragment which binds to human PD-1 comprising: a heavy chain comprising amino acid residues 20 to 466 of SEQ ID NO: 43 or a variant thereof, and/or a light chain comprising amino acid residues 20 to 237 of SEQ ID NO: 48 or a variant thereof.

In one embodiment, the invention provides an isolated antibody or antibody fragment which binds to human PD-1 comprising: a heavy chain comprising the amino acid residues 20 to 466 of SEQ ID NO: 43 or a variant thereof, and/or a light chain comprising the amino acid residues 20 to 237 of SEQ ID NO: 49 or a variant thereof.

In one embodiment, the invention provides an isolated antibody or antibody fragment which binds to human PD-1 comprising: a heavy chain comprising amino acid residues 20 to 466 of SEQ ID NO: 43 or a variant thereof, and/or a light chain comprising amino acid residues 20 to 237 of SEQ ID NO: 50 or a variant thereof.

In one embodiment, the invention provides an isolated antibody or antibody fragment which binds to human PD-1 comprising: a heavy chain comprising amino acid residues 20 to 469 of SEQ ID NO: 47 or a variant thereof, and/or a light chain comprising amino acid residues 20 to 237 of SEQ ID NO: 48 or a variant thereof.

In one embodiment, the invention provides an isolated antibody or antibody fragment which binds to human PD-1 comprising: a heavy chain comprising amino acid residues 20 to 469 of SEQ ID NO: 47 or a variant thereof, and/or a light chain comprising amino acid residues 20 to 237 of SEQ ID NO: 49 or a variant thereof.

In one embodiment, the invention provides an isolated antibody or antibody fragment which binds to human PD-1 comprising: a heavy chain comprising amino acid residues 20 to 469 of SEQ ID NO: 47 or a variant thereof, and/or a light chain comprising amino acid residues 20 to 237 of SEQ ID NO: 50 or a variant thereof.

In one embodiment, the variant(s) of the antibody or antibody fragment which binds to PD-1, preferably human PD-1, of the invention may comprise up to three, i.e., one, two or three conservate amino acid substitutions.

In one embodiment, the antibody or antibody fragment which binds to PD-1, preferably human PD-1, further comprises:
a. a human heavy chain constant region or a variant thereof, wherein the variant comprises up to 20, i.e., 1, 2, 3, 4, 5, 6, 7, 8, 9, 10, 11, 12, 13, 14, 15, 16, 17, 18, 19 or 20, conservative amino acid substitutions; and/or
b. a human light chain constant region or a variant thereof, wherein the variant comprises up to 20, i.e., 1, 2, 3, 4, 5, 6, 7, 8, 9, 10, 11, 12, 13, 14, 15, 16, 17, 18, 19 or 20, conservative amino acid substitutions.

In one embodiment, the human heavy chain constant region of the antibody or antibody fragment which binds to PD-1, preferably human PD-1, of the invention may comprise a γ4 or γ1 human heavy chain constant region or a variant thereof, wherein the variant comprises up to 20, i.e. 1, 2, 3, 4, 5, 6, 7, 8, 9, 10, 11, 12, 13, 14, 15, 16, 17, 18, 19 or 20, conservative amino acid substitutions.

In one embodiment, the antibody or antibody fragment which binds to PD-1 of the invention may:
a. bind human PD-1 with a KD of about 100 pM or lower;
b. bind human PD-1 with a KD of about 30 pM or lower;
c. bind to human PD-1 with about the same KD as an antibody having a heavy chain comprising the amino acid sequence of SEQ ID NO: 43 and a light chain comprising the amino acid sequence of SEQ ID NO: 44;
d. bind to human PD-1 with about the same KD as an antibody having a heavy chain comprising the amino acid sequence of SEQ ID NO: 43 and a light chain comprising the amino acid sequence of SEQ ID NO: 45;
e. bind to human PD-1 with a k_{assoc} of about 7.5×10⁵ 1/M·s or faster;
f. bind to human PD-1 with a k_{assoc} of about 1×10⁶ 1/M·s or faster;
g. bind to human PD-1 with a k_{dissoc} of about 2×10⁻⁵ 1/s or slower;
h. bind to human PD-1 with a k_{dissoc} of about 2.7×10⁻⁵ 1/s or slower;
i. bind to human PD-1 with a k_{dissoc} of about 3×10⁻⁵ 1/s or slower; and/or
j. block binding of human PD-L1 or human PD-L2 to human PD-1 with an IC₅₀ of about 1 nM or lower.

KD, k_{assoc} and k_{dissoc} values can be measured using any available method. In preferred embodiments, the dissociation constant is measured using bio-light interferometry (for example, the ForteBio Octet method described, e.g., in Example 2, page 45 of WO 2008/156712). In other preferred embodiments, the dissociation constant can be measured using surface plasmon resonance (e.g. Biacore) or Kinexa.

Further, in any of the embodiments, the antibody or antibody fragment of the invention may block binding of human PD-L1 or human PD-L2 to human PD-1 with an IC₅₀ of about 1 nM or lower. The blockade of ligand binding can be measured and the IC₅a calculated using any method known in the art, for example, the FACS or FMAT (fluorometric microvolume assay technology) methods described, e.g., in the Example 2, pages 46-47, section “Ligand Blockage” of WO 2008/156712.

The invention also comprises an antibody or antibody fragment which competes for a binding epitope on PD-1 with any of the antibodies of the invention targeting PD-1, preferably human PD-1, and has a one of the following characteristics:
a. binds human PD-1 with a KD of about 100 pM or lower;
b. binds human PD-1 with a KD of about 30 pM or lower;
c. binds to human PD-1 with about the same KD as an antibody having a heavy chain comprising the amino acid sequence of SEQ ID NO: 43 and a light chain comprising the amino acid sequence of SEQ ID NO: 44;
d. binds to human PD-1 with about the same KD as an antibody having a heavy chain comprising the amino acid sequence of SEQ ID NO: 43 and a light chain comprising the amino acid sequence of SEQ ID NO: 45;
e. binds to human PD-1 with a k_{assoc} of about 7.5×10⁵ 1/M·s or faster;
f. binds to human PD-1 with a k_{assoc} of about 1×10⁶ 1/M·s or faster;
g. binds to human PD-1 with a k_{dissoc} of about 2×10⁻⁵ 1/s or slower;
h. binds to human PD-1 with a k_{dissoc} of about 2.7×10⁻⁵ 1/s or slower;
i. binds to human PD-1 with a k_{dissoc} of about 3×10⁻⁵ 1/s or slower; and/or
j. blocks binding of human PD-L1 or human PD-L2 to human PD-1 with an IC₅₀ of about 1 nM or lower.

In one embodiment, the antibody or antibody fragment which binds to PD-1, preferably human PD-1, of the invention might be:
a. a chimeric antibody or a fragment thereof;
b. a human antibody or a fragment thereof;
c. a humanized antibody or a fragment thereof; and/or
d. an antibody fragment selected from the group consisting of Fab, Fab′, Fab′-SH, Fv, scFv, F(ab′)2, and a diabody.

In one embodiment, the antibody or antibody fragment which binds to PD-1, preferably human PD-1, of the invention may increase activation of T cells.

In one embodiment, the antibody or antibody fragment which binds to human PD-1 is a monoclonal and/or humanized antibody or fragments of monoclonal and/or humanized antibodies. In one embodiment, the antibody or antibody fragment which binds to human PD-1 is a chimeric antibody or fragments of chimeric antibodies. In one embodiment, the antibody which binds to human PD-1 is pembrolizumab (Keytruda) or fragments thereof. In another embodiment, the antibody which binds to EGF receptor, specifically to ERBB2, is trastuzumab (Herceptin) or fragments thereof.

The preparation of chimeric, human, humanized or monoclonal antibodies as well as antibody purification methods are known in the art and can be prepared as described, e.g., in WO 2008/156712 which is incorporated herein in full, particularly with regard to pages 27-31.

In a further preferred aspect of the invention, the agent effective in the treatment or prevention of an esophageal disease is an antiproliferative agent.

An “antiproliferative agent as used herein” refer to a substance which prevents, blocks, reduces, decreases, or inhibits cell proliferation and/or cell growth, particularly of cancer cells. The term also relates to agents active in the prevention, reduction, decrease or inhibition of the spreading of cells, particularly malignant cells, into the surrounding tissue. Thus, the antiproliferative agent may interfere with the formation of metastases. The term may further relate to agents that kill cells, particularly cancer cells. The agents include but are not limited to chemotherapeutic agents such as alkylating drugs, antimetabolites, anti-tumor antibiotics, plant alkaloids, plant taxanes, platin-based agents, or steroid hormones. The antiproliferative agents as described herein are known in the art and can be prepared according to any known method. The antiproliferative agents as described herein are also commercially available.

In one embodiment, the antiproliferative agent is selected from the group consisting of a taxane; a pyrimidine analogue, and a platin-based agent.

Taxanes belong to the class of diterpenes. They have been originally identified from natural sources, e.g., plants of the genus Taxus. Drug formulation of taxanes is difficult due to their poor water solubility. In principle, any taxane can be used for the purpose of the invention including but limited to isolated natural taxanes, naturally modified taxanes, e.g., semi-synthesized taxanes, or taxanes of the first and/or second generation. Taxanes are mitotic inhibitors and are also known as spindle poisons. They inhibit the process of cell division by disruption of microtubule function in that they prevent depolymerization of microtubules. In one embodiment, the taxane is selected from the group consisting of paclitaxel, docetaxel, and cabazitaxel, preferably paclitaxel. In one embodiment, the taxane is present in a carrier, e.g., a liposome, preferably a cationic liposome.

Pyrimidine analogues are heterocyclic organic compounds based on a pyrimidine. Pyrimidine analogous are used in cancer therapy due to their antimetabolite activity. Specifically, pyrimidine analogues act as building blocks of DNA and interfere with DNA production either by incorporation chemically altered nucleotides in the growing DNA chain or be depleting the supply of deoxynucleotides needed for DNA proliferation and DNA replication. They stop normal cell division and interfere with tumor growth because tumor cells spend more time in cell division than other cells. Thus, tumor cells are particularly affected by pyrimidine analogues. In a preferred embodiment, the pyrimidine analogue is an uracil analogue, preferably 5-flurouracil or capecitabin. In one embodiment, the pyrimidine analogue is present in a nucleic acid carrier system as described herein.

Platinum-based agents are coordination complexes of platinum and are used to treat almost half the patients suffering from cancer. Without being bound by any theory, platinum-based agents cause crosslinking of DNA, and the resulting crosslinking inhibits DNA repair and/or DNA synthesis. In a preferred embodiment, the platinum-based agent is selected from the group consisting of cisplatin or a salt thereof, carboplatin or a salt thereof, nedaplatin or a salt thereof, and oxaliplatin or a salt thereof. The platinum-based agent might be further selected from the group consisting of triplatin tetranitrate or a salt thereof, phenanthriplatin or a salt thereof, picoplatin or a salt thereof and satraplatin or a salt thereof. The platinum-based agent might also comprise platinum salts, preferably a cisplatin salt, a carboplatin salt or an oxaliplatin salt.

The present invention provides a drug delivery system comprising an agent effective in the treatment or prevention of an esophageal disease as described herein. The drug delivery system described herein may also include one or a combination of (e.g., two or more different) agent(s) effective in the treatment or prevention of an esophageal diseases as described herein or further agent(s) effective in the treatment or prevention of an esophageal diseases.

For example, an inhibiting polynucleotide, antibody or antibody fragment or an antiproliferative agent of the invention can be combined with a treatment that is considered to be standard of care in cancer. Rationale for such combinations is that the combination will induce or facilitate initial clinical response to standard of care treatment, induce durable clinical response and long-term immune control of disease.

In one embodiment, treatment with an inhibiting polynucleotide, antibody or antibody fragment of the invention may be combined with chemotherapy. Chemotherapy will result in cancer cell death thereby increasing release of tumor antigens. Such increased availability of tumor antigen may result in synergy with treatment with inhibiting polynucleotide, antibody or antibody fragment of the invention. A non-limiting example is provided by combining the inhibiting polynucleotide, antibody or antibody fragment or the antiproliferative agent with the antiproliferative agents of the invention. In one embodiment, treatment with an antiproliferative agent of the invention may be combined with chemotherapy, i.e., an antiproliferative agent which is different from the antiproliferative agent of the invention to provide a synergistic effect.

In one embodiment, treatment with an inhibiting polynucleotide, antibody or antibody fragment or an antiproliferative agent of the invention may be combined with radiotherapy. Radiotherapy induces cancer cell death and increasing availability of tumor antigens for presentation and activation of immune cells. In another embodiment, treatment with an inhibiting polynucleotide, antibody or antibody fragment or an antiproliferative agent of the invention may be combined with surgery to remove cancer cells from a subject.

### Additional Active Pharmaceutical Ingredients (API)

The API within the present dosage form may be administered together with an additional API.

Additional APIs that might be present in addition to the agent effective in the treatment or prevention of an esophageal disease are referred to herein as “additional active pharmaceutical ingredient” or “additional active ingredient” or “additional API”. In principle, any additional pharmaceutical active agent may be used which enhances or increases the efficacy of the agent effective in the treatment or prevention of an esophageal disease. Such additional APIs may be selected from the skilled person based on his or her general knowledge depending upon the condition to be treated and/or prevented.

For example, the invention also comprises an immunoconjugate comprising an antibody or antibody fragment of the invention, linked to a therapeutic agent such as a bacterial toxin, an antiproliferative agent or a radiotoxin. Non-limiting examples of cytotoxic agents include taxol, cytochalasin B, mitomycin, etoposide and vincristine or other antimetabolites, alkylating agents, antibiotics and antimitotics. As used herein, an “immunoconjugate” refers to an antibody, or a fragment thereof, conjugated to a therapeutic moiety, such as a bacterial toxin, a cytotoxic drug, an antiproliferative agent or a radiotoxin. Toxic moieties can be conjugated to antibodies of the invention using methods available in the art.

In some embodiments, an inhibiting polynucleotide, antibody or antibody fragment or an antiproliferative agent of the invention may be combined with a second therapeutic agent or treatment modality. In one embodiment, an inhibiting polynucleotide, antibody or antibody fragment or an antiproliferative agent of the invention may be combined with cancer treatments involving the application of recombinant cytokines or secreted immune factors or further antiproliferative agents such as taxanes, pyrimidine analogues, e.g., fluoropyrimdine analogues or platin-based agents. Non-limiting examples of combinations include combining inhibiting polynucleotides, antibodies or antibody fragments or antiproliferative agents of the invention with recombinant IL-2, chemotherapeutic or recombinant EFNα2. Recombinant IL-2 enhances T cell outgrowth in cancer patients. Recombinant EFNα2 inhibits cancer cell growth but also increases expression of the inhibitory ligands for PD-1 on cancer cells, antigen-presenting cells and other somatic cells in the treated patients. The inhibiting polynucleotides, antibodies or antibody fragments or antiproliferative agents of the invention can be combined with other cytokines that might be considered useful for the treatment of cancer.

In a specific embodiment, the antibody or a binding fragment thereof used in the present invention, e.g., an antibody which targets a polypeptide encoding PD-1, preferably an antibody which binds to human PD-1 such as pembrolizumab (Keytruda) or fragments thereof or an antibody which binds to the EGF-receptor, preferably trastuzumab, or fragments thereof, is combined with a platinum-based and/or fluoropyrimidine-based antiproliferative agent for the treatment of an esophageal disease or disorder. In one embodiment, the platinum-based antiproliferative agent is e.g., cisplatin. In one embodiment, the fluoropyrimidine-based antiproliferative agent is 5-fluorouracil and/or capecitabin. In one embodiment, the esophageal disease or disorder is esophageal cancer such as esophageal squamous cell carcinoma, preferably progressed or metastatic esophageal squamous cell carcinoma, more preferably esophageal squamous cell carcinoma with PD-L1 expressing tumors, or esophageal adenocarcinoma/esophageal junction carcinoma, preferably progressed or metastatic esophageal adenocarcinoma, more preferably esophageal adenocarcinoma with PD-L1 expressing tumors which may be HER-2 positive or HER-2 negative. In a preferred embodiment, the treatment is a first-line treatment.

### Preparation

In a preferred embodiment of the drug delivery system according to the present invention the sheet like preparation is a wafer or is formed as a wafer. The term “wafer” as uses herein, refers to a sheet, which comprises several layers used to enclose the agent effective in the treatment or prevention of an esophageal disease.

Such a wafer can fit to the irregular surface contour of a predetermined site of action, in particular of the esophageal mucous membrane, in particular after absorption of moisture contained in the esophageal mucous membrane by the wafer. Additionally, a sheet like preparation of a dosage form according to the invention may be gellable or swellable.

In a preferred embodiment of the drug delivery system according to the present invention the thickness of the sheet like preparation is 0.01 mm to 2 mm, preferably 0.03 mm to 1 mm, preferably 0.05 mm to 0.1 mm. This is beneficial to provide a sheet like preparation with a relatively small thickness.

In a preferred embodiment of the drug delivery system according to the present invention the sheet like preparation has an area between 0.5 and 25 cm2, preferably between 1 to 10 cm2.

The sheet like preparation may have different shapes. In particular, a sheet like preparation can have a round, triangular, quadrangular or polygonal shape. In an embodiment, the aperture is adapted to fit the respective shape of the preparation.

In a preferred embodiment of the drug delivery system according to the present invention the sheet like, in particular film shaped, foil shaped, or wafer shaped, preparation, that comprises the agent effective in the treatment or prevention of an esophageal disease, contains an agent effective in the treatment or prevention of an esophageal disease with a drug content of 0.0001 to 50% by weight, preferably 0.001 to 25% by weight, and most preferred 0.01 to 10% by weight.

The sheet like preparation comprising the agent effective in the treatment or prevention of an esophageal disease may have a single-layered or multi-layered structure, wherein at least one (preferably first) layer contains the agent effective in the treatment or prevention of an esophageal disease.

In a preferred embodiment the sheet like preparation has a multi-layered structure of multiple layers, wherein at least a first layer contains the agent effective in the treatment or prevention of an esophageal disease and wherein at least a further layer contains at least one further active pharmaceutical ingredient, which is either the same or a different agent effective in the treatment or prevention of an esophageal disease or which is not an agent effective in the treatment or prevention of an esophageal disease, such as a steroid.

In a preferred embodiment the layer containing the agent effective in the treatment or prevention of an esophageal disease and/or the further layer containing the additional active pharmaceutical ingredient comprises a polymer, preferably a film forming polymer.

The polymer within the layer may serve merely as a carrier for the agent effective in the treatment or prevention of an esophageal disease and/or the additional API, or it may serve as a reservoir for same. Such a layer can release the agent effective in the treatment or prevention of an esophageal disease and/or the additional active pharmaceutical ingredient under the effect of a fluid. The agent effective in the treatment or prevention of an esophageal disease and/or the additional API may be released immediately or in a controlled release manner.

In a preferred embodiment of the drug delivery system according to the present invention the sheet like preparation comprises at least a first layer containing an agent effective in the treatment or prevention of an esophageal disease and/or a further layer containing an agent effective in the treatment or prevention of an esophageal disease and/or additional API, wherein the at least one first layer and/or the further is an adhesive layer.

In a preferred embodiment of the drug delivery system according to the present invention the at least one first layer containing the active ingredient and/or the further layer containing the active ingredient comprises a polymer, preferably a film forming polymer, wherein the polymer is a film forming polymer that is water dispersible and/or decomposable and/or water disintegrable.

A polymer for a first layer containing an active substance and/or for a further layer containing an active substance may, in particular, be selected from a group comprising polyvinyl alcohols, Polyvinylpyrrolidone, polyvinyl acetate, polyethylene glycol, polyethylene oxide polymers, polyurethanes, polyacrylic acids, polyacrylates, polymethacrylates, poly (methyl vinyl ether-maleic acid anhydrides), starch, starch derivates, natural gums, alginates, pectins and gelatin, Pullulan, gel forming proteins, Chitosan, Agar-Agar, agarose, carrageenan, xanthan, tragacanth, dextran, and cellulose ethers such as ethyl cellulose, hydroxyethyl cellulose, propyl cellulose, carboxymethyl cellulose, sodium-carboxy methylcellulose, hydroxypropyl cellulose, hydroxypropyl methylcellulose, hydroxypropyl ethyl cellulose, cellulose acetate, povidone and copovidone. In a preferred embodiment, the polymer is a polyvinyl alcohol, preferably polyvinyl alcohol 18-88.

The polymers may be used individually or in a combination with each other to manufacture a sheet like preparation for the dosage form according to the invention with the desired properties as adhesion, release or disintegration properties. A sheet like preparation according to the invention may consist of a single polymer layer.

Also, a sheet like preparation for a dosage form according to the invention may have a structure with two or multiple layers, when at least one of the layers contains an agent effective in the treatment or prevention of an esophageal disease optionally in combination with a nucleic acid delivery system and/or further excipients for stabilizing the agent effective in the treatment or prevention of an esophageal disease. It is also possible that multiple layers contain either an agent effective in the treatment or prevention of an esophageal disease or an additional API.

In a preferred embodiment of the drug delivery system according to the present invention, the sheet like preparation comprising an antiproliferative agent comprises or consists of a single-layered structure, wherein a (preferably first) layer contains, preferably is coated with, the antiproliferative agent, preferably paclitaxel. The layer comprises a polymer, preferably a film forming polymer, wherein the polymer is a film forming polymer that is water dispersible and/or decomposable and/or water disintegrable. The polymer is a polymer as described herein, preferably polyvinyl alcohol, preferably polyvinyl alcohol 4-88 or polyvinyl alcohol 18-88. The layer further comprises additives as described herein, such as a plasticizer, preferably glycerol and a surfactant, preferably a lauryl alcohol such as an ethoxylated lauryl alcohol. Such surfactants are commercially available and include but are not limited to Brij™L23. The purpose of the surfactant is to provide further stability by reducing hydrolysis and adsorption to unwanted materials.

In a further preferred embodiment of the drug delivery system according to the present invention, the sheet like preparation comprising an inhibiting polynucleotide comprises or consists of a single-layered structure, wherein a (preferably first) layer contains, preferably is coated with, the inhibiting polynucleotide, preferably an siRNA, antisense oligonucleotide or aptamer, optionally in combination with a nucleic acid delivery system. The layer comprises a polymer, preferably a film forming polymer, wherein the polymer is a film forming polymer that is water dispersible and/or decomposable and/or water disintegrable. The polymer is a polymer as described herein, preferably polyvinyl alcohol, preferably polyvinyl alcohol 18-88. The layer further comprises additives as described herein, such as a plasticizer, preferably glycerol and/or stabilizers, preferably sucrose.

In a further preferred embodiment of the drug delivery system according to the present invention, the sheet like preparation comprising an antibody comprises or consists of a single-layered structure, wherein a (preferably first) layer contains, preferably is coated with, the antibody. The layer comprises a polymer, preferably a film forming polymer, wherein the polymer is a film forming polymer that is water dispersible and/or decomposable and/or water disintegrable. The polymer is a polymer as described herein, preferably polyvinyl alcohol, preferably polyvinyl alcohol 18-88.

In another preferred embodiment of the drug delivery system according to the present invention, the sheet like preparation comprising an agent effective in the treatment or prevention of an esophageal disease comprises at least one first active ingredient free layer that does not contain an active pharmaceutical ingredient.

In a preferred embodiment of the drug delivery system according to the present invention, the sheet like, in particular film shaped, foil shaped, or wafer shaped, preparation comprising the active pharmaceutical ingredient comprises at least a further active ingredient free layer that does not contain an active pharmaceutical ingredient.

In a preferred embodiment of the drug delivery system according to the present invention the first active ingredient free layer and/or the at least one further active ingredient free layer is a water insoluble layer which preferably comprises water insoluble substances selected from the group ethyl cellulose and/or combinations of ethyl cellulose with other water insoluble substances, hydrophobic plasticizers, especially triethyl citrate, and/or dies and/or fragrances and/or flavorings.

In particular, the use of ethyl cellulose may be beneficial due to its properties comprising a good processability, biocompatibility, and water insolubility.

In a preferred embodiment of the drug delivery system according to the present invention the first active ingredient free layer and/or the at least one further active ingredient free layer is an adhesive layer of desired thickness.

The adhesive layer may be a mucoadhesive polymer selected from the group comprising cellulose derivates, such as hydroxypropyl cellulose, starch, and starch derivates, polyvinyl alcohol, polyethylene oxide, polyethylene, polypropylene, polyacrylic acid and polyacrylate derivates, polyvinylpyrrolidone, povidone, copovidone, sodium alginate, gelatin, xanthan gum, carrageenan, pectins, dextrans, lectins, chitosan, pullulan, and mixtures thereof.

Additionally, or alternatively, the adhesive layer may comprise a solvent that is selected from the group comprising water, ethanol, methanol, acetone, organic solvents, and mixtures thereof.

Furthermore, the preparation may additionally contain additives such as colorants, fragrances, flavoring agents, preservatives, antioxidants, penetration enhancers, solubilizers, disintegration accelerators, pore formers, lubricants, stabilizers and mixtures thereof. In particular, the following substances are eligible as additives: lubricants, lubricants, glidants, binders, additional active ingredients, disintegrants, antioxidants, chelating agents, coating agents, flow agents, preservatives, fillers, surfactants, plasticizers, stabilizers, and pigments. Furthermore, the additives may be selected from the following group: pore formers, penetration enhancers, solubilizers, emulsifiers, comprising polyethoxylated sorbitan fatty acid esters, ethoxylated fatty alcohols, and lecithin; plasticizers, comprising polyethylene glycol, glycerol and other polyhydric alcohols, higher alcohols such as dodecanol, undecanol, or octanol, sorbitol, mannitol and other sugar alcohols, dexpanthenol and triglycerides; fillers comprising highly disperse silicon dioxide, titanium dioxide, zinc oxide, chalk and starch; colorants; sweetening and flavoring agents; wetting agents; preservatives; pH regulators and antioxidants; disintegration accelerators; penetration enhancers which improve the resorption of the active pharmaceutical ingredient into the mucous membrane, e.g. the cellular uptake, for example, fatty acids and salts thereof and fatty acid esters, preferably saturated fatty acids such as octanoic acid (C8), decanoic acid (C10), octadecanoic acid (C18), or unsaturated fatty acids such as oleic acid (C18), salcaprozate (SNAC) or a salt thereof, terpenes, glycolipids, medium-chained triglycerides, synthetic waxes such as isopropyl myristate, branched fatty alcohols such as Eutanol G®, urea, polypropylene glycol, dimethyl sulfoxide, azones, azone analogs, polyhydric alcohols such as propanediol, tocopherols or essential oils such as menthol. A preferred plasticizer is glycerol.

The sheet-like preparation may further comprise at least one taste-masking additive. This advantageously allows the masking of a bitter or in some other way unpleasant tasting active pharmaceutical ingredient but may also be beneficial to accelerate the onset of effect of an active pharmaceutical ingredient. Taste-masking additives are known to the person skilled in the art. Such a taste-masking additive may, in particular, comprise a sugar alcohol selected from mannitol, sorbitol, xylitol, malitol, lactitol, erythritol, threitol, and isomalt as well as sodium hydrogen carbonate.

In particular, the additives may improve the local availability of the active ingredient, such as penetration enhancers.

According to a preferred embodiment the drug delivery system, in particular the sheet like preparation, according to the invention is intended to enable a time delayed active ingredient release. The agent effective in the treatment or prevention of an esophageal disease is preferably released over a period of 4 hours, preferably over a period of 6 hours and most preferably over a period of 8 hours. In order to achieve a delayed active ingredient release in case of two-layered or multi-layered preparations, at least one of the layers containing the agent effective in the treatment or prevention of an esophageal disease, in particular a polymer layer, has a delayed active ingredient release.

For a delayed active ingredient release the film shaped medicaments are preferably formulated as slowly soluble or slowly disintegrating film which are completely disintegrated or dissolved only after several hours. Preferably, they are completely disintegrated or completely dissolved only after 4 hours, preferably only after 6 hours, and even most preferably only after 8 hours or even only after 24 hours.

In particular, the agent effective in the treatment or prevention of an esophageal disease and the optionally present additional API are released within a period of 15 minutes to 24 hours, 2 hours to 24 hours, 3 hours to 12 hours, 4 hours to 8 hours, or 5 to 6 hours.

The sheet like preparation can be prepared by a person skilled in the art by basically known methods, for example by coating of an inert support with a liquid composition which comprises the polymer(s), agent effective in the treatment or prevention of an esophageal disease/additional active pharmaceutical ingredient(s) and optionally additive(s) and solvent(s), by means of e.g., a method involving a doctor blade (e.g., solvent casting), spray processors or extrusion processors. The thin film layer obtained in such a way is dried. For a multi-layered sheet like preparation one or more coatings may be applied onto the existing film layer in the same manner or may be manufactured separately and then be subsequently laminated.

During manufacture of the preparation, the temperature-sensitivity, pH-sensitivity, enzymatic stability and/or solubility of the used agent effective in the treatment or prevention of an esophageal disease may be required to be taken into consideration. Of course, the specific properties of the active pharmaceutical ingredient should also be considered during the whole production process of the drug delivery device. Thus, and in addition in view of the low dosages needed, impregnation processes may be used. In such processes which are preferred in the present invention, a solution comprising the agent effective in the treatment or prevention of an esophageal disease is merely applied, e.g., sprayed or dripped, onto a polymer film, which is ultimately dried. Such methods are known to the skilled person and are described in the examples provided herein.

In another example, the agent effective in the treatment or prevention of an esophageal disease may be incorporated such that it is embedded in the polymer film, e.g., by solvent casting.

In all these processes care should be taken with regard to the solvent used, and the drying conditions. As a very elegant drying method freeze-drying may be used.

Furthermore, depending on the stability of the agent effective in the treatment or prevention of an esophageal disease incorporated also melt extrusion of polymer and agent effective in the treatment or prevention of an esophageal disease are imaginable, e.g., as described in Example 5 herein. Alternatively, the solution comprising the agent effective in the treatment or prevention of an esophageal disease solution may be applied to the polymer film via an inkjet process.

In one embodiment, the preparation is manufactured such that the agent effective in the treatment or prevention of an esophageal disease is only present is certain portions within the film, which would allow a tailor-made treatment of the mucosa in designated areas only.

Alternatively, and preferably, a first region of the sheet like preparation may be in contact with an esophageal mucosa and a second region of the sheet like preparation may be in contact with a buccal mucosa. In this way, the esophageal mucosa can be treated with the agent effective in the treatment or prevention of an esophageal disease while the buccal mucosa is treated with a second the agent effective in the treatment or prevention of an esophageal disease, an additional API, not treated or an additive is released to the buccal mucosa. In particular, a flavoring agent and/or a local anesthetic may be released, particularly to increase or decrease the production of saliva and/or to make the application of the drug delivery system more pleasant and/or to suppress the urge to gag. Alternatively, the first region of the sheet like preparation may be in contact with an esophageal mucosa and the second region of the sheet like preparation may be in contact with the mucosa of the upper section of the stomach, such as the cardia, or the cardia and the fundus. It would therefore be possible to treat the esophagus and parts of the stomach locally.

In another preferred embodiment, the drug delivery system, in particular the capsule device, comprises a sinker device. The sinker device is configured to provide negative buoyancy to the capsule device. In experiments of the inventors underlying the finding of this preferred embodiment it was found that reducing the buoyancy, for example by increasing the mass of the capsule device, leads to an improved reliability of the mechanical process of expanding the preparation from the compacted condition to the expanded condition. In case of strip-like preparation, the unwinding of the preparation from the compacted condition, where the strip-like preparation is wound around a winding axis, to the expanded condition was significantly facilitated and more efficient. The problem underlying the preferred embodiment is the observation that the transfer of the preparation from the compacted condition to the expanded condition is sometimes incomplete. While the invention already improves the efficiency of expansion, or respectively, unwinding, by providing a spacing between the opening and the preparation, the sinker device additionally increases the efficiency of expansion. It is assumed that the capsule device, also if properly swallowed by a patient in the presence of water or aqueous solution, is not completely filled with water but air-bubbles sometimes remain inside the capsule device. The air contributes to buoyancy, and the sinker device assists to resist the buoyancy effects by assisting in the displacement of air or by using denser materials than water for utilizing gravity. Further details regarding the sinker are to be deduced from WO2020/183005, which is incorporated herein by reference.

A preferred embodiment of the drug delivery system according to the present invention is adapted for the application to a nasopharyngeal mucosa.

When the sheet like preparation releases the agent effective in the treatment or prevention of an esophageal disease, optionally together with an additional API, locally and/or over a prolonged time, the therapeutic response may be improved, and in particular the local effect of the agent effective in the treatment or prevention of an esophageal disease can be increased by e.g., a penetration enhancer. Such penetration enhancers are known in the art. Furthermore, in particular due to the spatially extended region of action, the necessity for a systemic administration may be reduced.

In a preferred embodiment of the drug delivery system according to the present invention the sheet like preparation has an area and/or surface area between 0.5 and 25 cm², preferably between 2 to 25 cm², preferably between 5 to 25 cm², preferably between 5 to 15 cm², preferably larger than 0.5 cm², and preferably smaller than 40 cm². Preferably the ration of the length of the sheet like preparation and the width of the sheet like preparation is between 40:1 and 400:1, or preferably 60:1 and 300:1, or preferably 80:1 and 200:1. Said width can be an average width of the sheet like preparation, measured, for example, perpendicular to the length of the sheet like preparation. Said ratio can be a ratio of the length of the sheet like preparation and a circumference, in particular an average, of the sheet like preparation, wherein said circumference can be, for example, twice the width of a sheet like preparation in the case of a strip-shaped sheet like preparation.

In certain embodiments of the drug delivery system according to the present invention the sheet like preparation is in a solid-state, in particular while it is in its compact form and/or immediately after its release. This may beneficially enhance, enable or facilitate some of the above-mentioned advantages. In particular, this may enhance the storability, when it is in a solid state prior the release. In particular, this may enhance and/or enable a targeted and/or sustained release of the agent effective in the treatment or prevention of an esophageal disease, when it is in a solid state after its release. Additionally, or alternatively, in certain embodiments of the drug delivery system according to the present invention, the sheet like preparation is adapted to dissolve, e.g., bio-degenerate, immediately, after a delay, in a time-controlled manner or upon a stimulus after its release. This may beneficially enhance, enable, or facilitate some of the above-mentioned advantages. In particular, this can improve the user convenience, because the sheet like preparation does not need to be removed.

Additionally, or alternatively, in certain embodiments of the dosage form according to the present invention the sheet like preparation is adapted to dissolve, e.g., to bio-degenerate, preferably in a time-controlled manner, e.g., within one hour, or within one to two hours or within one to five hours, or within one to twelve hours, or within one to twenty-four hours. This improves the user convenience as the sheet like preparation does not need to be removed.

### Applicator/Retainer

In an embodiment, an applicator with a holder serves to assist swallowing the capsule device in combination with a drinking cup. The applicator in combination with the drinking cup allows the patient to take the drug delivery system as if drinking from a bottle. The applicator is therefore mounted on the drinking cup as a mouthpiece. The drug delivery system is positioned in the holder of the applicator. When drinking, the liquid of the drinking cup is rinsed through the applicator and the holder inside, which releases the preparation from the holder and transports it into the mouth of the patient, who then swallows it. The string member is a retainer and is wound around the holder.

The string member is further connected to the holder and to the end of the preparation, which extends through the aperture. Thus, when the preparation leaves the holder during drinking, the holder is unwound until it is taut. This then exerts a force on the preparation, pulling the preparation out of the capsule.

Such an applicator and drinking cup is, for example, described in PCT/EP2020/056927, which is incorporated by reference herein in full, with regard to an applicator, a drinking cup and a string. Such a retainer is further described, for example, in EP21175427.0 and EP21175436.1, which are incorporated by reference herein in full, with regard to a retainer.

In a preferred embodiment, the retainer is wrapped around a support structure of the holder, whereas one end of the retainer is attached to the support structure and the other end is connected to the preparation of the capsule device. The capsule device is therefore positioned and hold inside the holder of the applicator. When the patient swallows the dosage form, the retainer begins to unwind from the support structure. The applicator and the support structure have a cylindrical shape so that the support structure fits into the applicator and, in particular, is rotatably mounted therein so that the retainer can unwind from the structure by rotating the structure.

This is particularly beneficial, if the dosage form is to be administered on a regular, in particular daily, basis as administration of the capsule device is then possible without professional help.

In a preferred embodiment of the drug delivery system according to the present invention, the release mechanism comprises the retainer, which preferably is a string, wherein the string is expandable from a compact form to an expanded form and connected to an end of the preparation which protrudes out of the capsule device.

Exemplary embodiments of the present invention will be described in greater detail below with reference to the accompanying drawings and samples, from which further features, advantages, and embodiments can be learned.

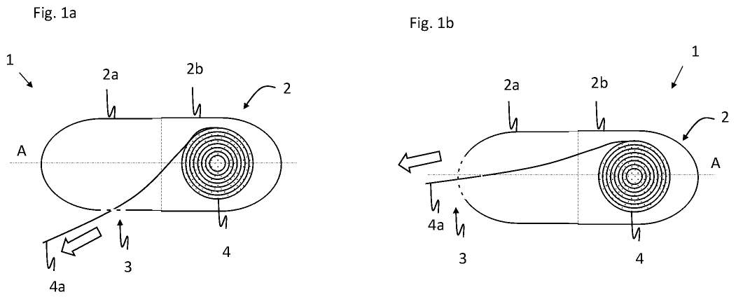

FIGS. 1a, 1b each show schematic illustrations of a capsule device of the drug delivery system.

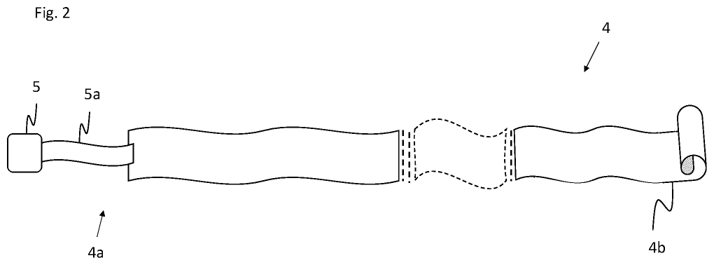

FIG. 2 shows a schematic illustration of a preparation in its partially unfolded form.

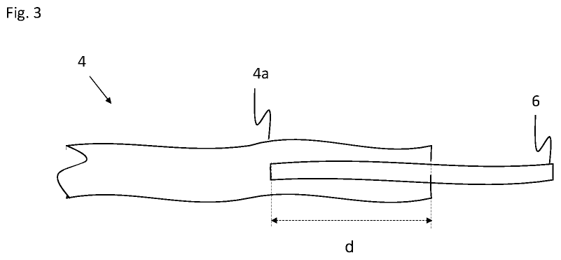

FIG. 3 shows a schematic illustration of the one end of a preparation which is connected to a retainer for pulling the preparation out of the capsule device.

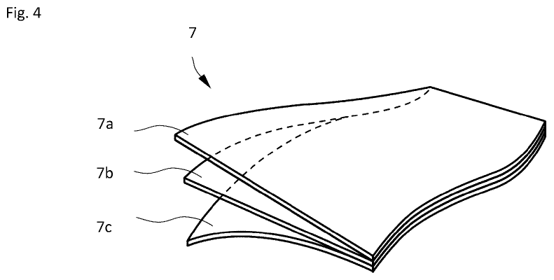

FIG. 4 shows a schematic illustration of the preparation being a three-layered wafer.

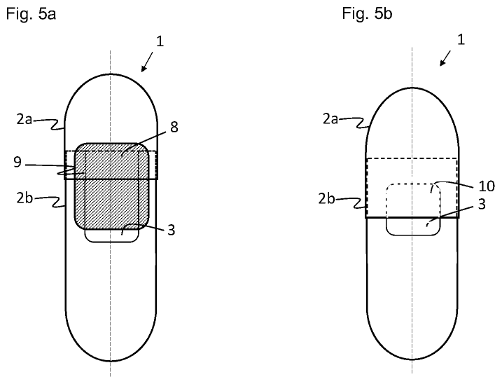

FIGS. 5a, 5b each show schematic illustrations of a capsule device having an aperture formed by an overlapping wall part or by telescoping two capsule halve-shells into each other.

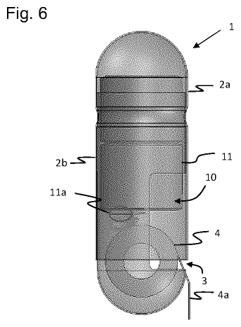

FIG. 6 shows a schematic semi-transparent view of a drug delivery system.

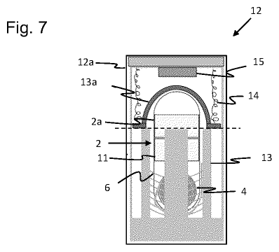

FIG. 7 shows a schematic illustration of an applicator, with a holder and a retainer wound around the holder, whereas a drug delivery system is positioned inside the holder.

FIGS. 8a, 8b each show a schematic view of a patient taking the drug delivery system using the applicator and drinking cup, before (FIG. 8a) and during (FIG. 8b) swallowing of the drug delivery system.

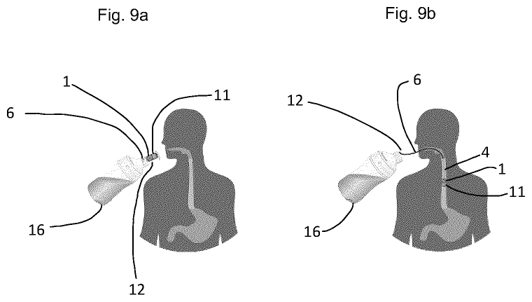

FIG. 9 show a schematic view of a patient taking a specific embodiment of the drug delivery system including a sinker using the applicator and drinking cup, before (FIG. 9a) and after (FIG. 9b) swallowing of the drug delivery system.

FIG. 10a shows the structure of the fluorescent dye Atto633. FIG. 10b shows the structure of ATTO633 and the chemical linkage to RNA: The dotted line indicates the attachment site to the ribonucleic acid.

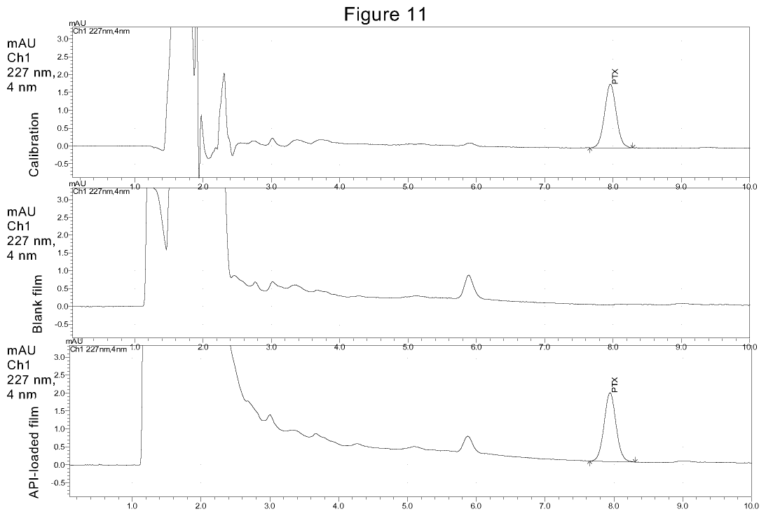

FIG. 11 shows the results of HPLC with a calibration sample, blank film sample (comprising no paclitaxel) and an API-loaded film sample (comprising paclitaxel). Shown is milli absorption unit (mAU) against the retention time in minutes.

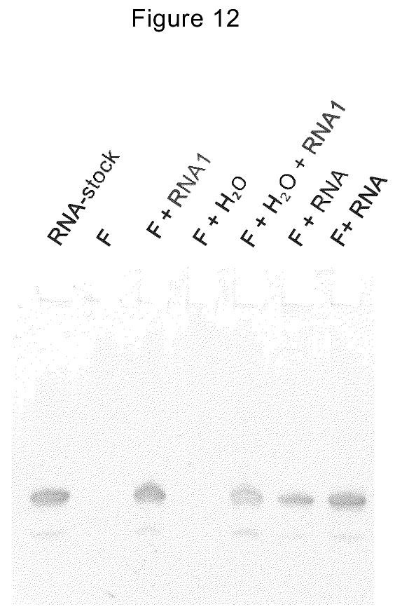

FIG. 12 shows the influence of the film on RNA stability. RNA was analyzed on a 20% polyacrylamide gel. “RNA-stock” means pure synthesized RNA, “F” means film, “RNA1” means RNA addition after the film was solubilized and “RNA” means RNA applied on the dry film.

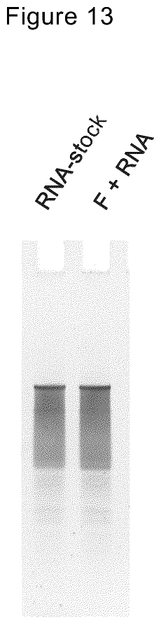

FIG. 13 shows the reproducibility of the stability results obtained with synthesized RNA with commercially available siRNA. “RNA-stock” means pure commercial RNA sample, “F+RNA” means RNA applied on the dry film.

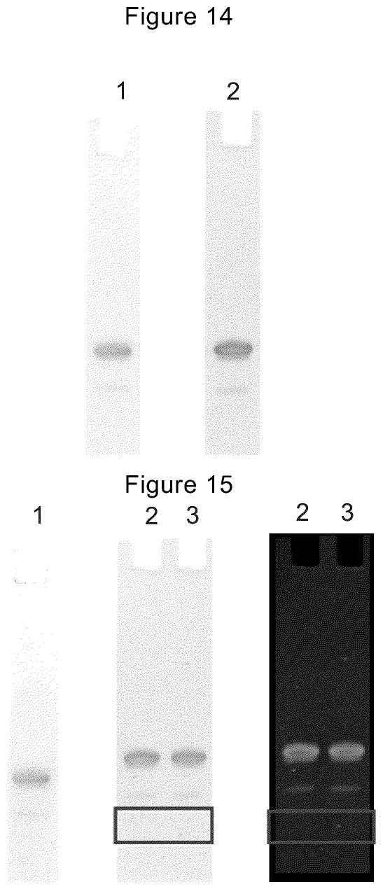

FIG. 14 shows the storage stability of RNA on a dry film. “1” refers to analysis directly after samples have been dried, “2” refers to analysis of dried samples after 4 days at 4° C.

FIG. 15 shows the storage stability of RNA on a solubilized film. “1” refers to analysis directly after solubilization, “2” refers to analysis of solubilized samples after 4 days at 4° C. and “3” refers to analysis of solubilized samples after 4 days at 4° C. in the presence of RNAse inhibitor. The box indicates position of slight degradation of RNA as shown in column 2 which could be prevented in the presence of RNAse inhibitor as shown in column 3.

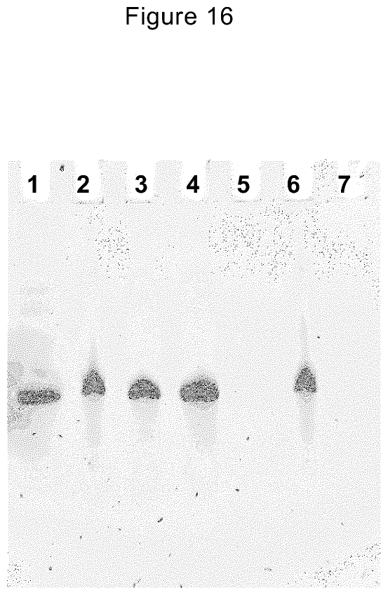

FIG. 16 shows the stability of antisense BMP2 RNA on the film under various application conditions. “1” refers to the stock solution, i.e., the RNA solution prior to application onto the film, “2” refers to a sample in which the film was wet when applying the RNA onto it, “3” refers to a sample in which the film was dry when applying the RNA onto it, “4” refers to a sample in which water was applied on the film and RNA was applied after the film has dissolved, “5” refers to a sample in which water was applied on the film, “6” refers to a sample in which RNA was applied on the film after the film has dissolved, and “7” refers to a sample in which the film has dissolved. For samples 2 to 6, 0.5 cm² of the film has been solved in 75 μl water.

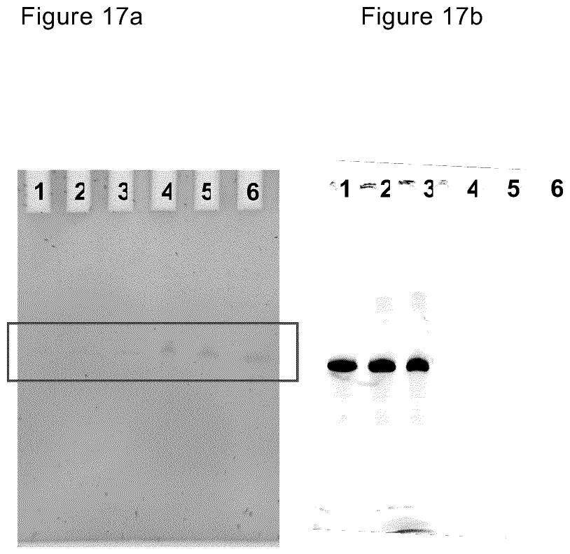

FIG. 17 shows the stability of Atto633-labeled antisense BMP2 RNA in comparison to unlabeled antisense BMP2 RNA on the film under various application conditions. FIG. 17a ethidium bromide staining of the RNA. The box indicates the position of the RNA on the gel. FIG. 17b shows the fluorescence of labeled RNA. “1” refers to a sample in which the film was dry when applying the labeled RNA onto it, “2” refers to a sample in which the film was wet when applying the labeled RNA onto it, “3” refers to a sample of labeled RNA, “4” refers to a sample in which the film was dry when applying the unlabeled RNA onto it, “5” refers to a sample in which the film was wet when applying the unlabeled RNA onto it, and “6” refers to a sample of unlabeled RNA. For samples 2 to 6, 0.5 cm² of the film has been solved in 75 μl water.

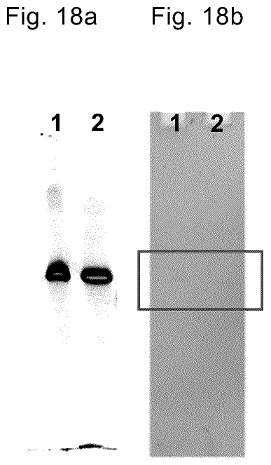

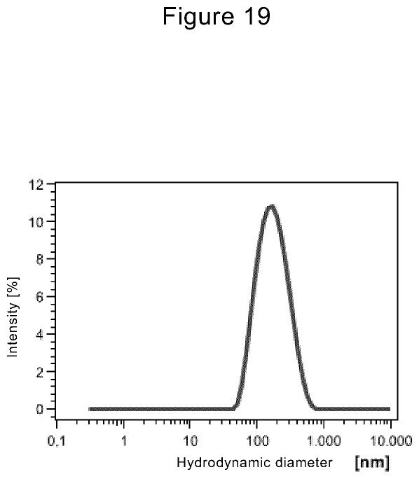

FIG. 18 shows the stability of labeled and unlabeled BMP2 RNA after application on the film and subsequent storage for 10 days at 4° C. FIG. 18a shows the fluorescence of labeled RNA. FIG. 18b shows ethidium bromide staining of the RNA. “1” refers to a sample in which the labeled BMP2 RNA was applied, and the film was dissolved, “2” refers to a sample of labeled BMP2 RNA. For sample 1, 0.5 cm² of the film has been solved in 75 μl water.

FIG. 19 shows the state of the starting product before drying. FIG. 19 is a graphical representation of the results of liposome sizing.

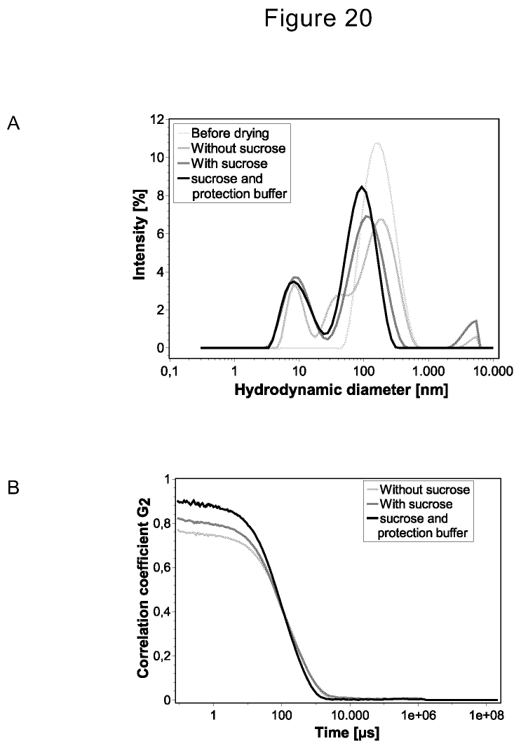

FIG. 20 shows the state of liposomes after drying on different film compositions. Figure A shows the curves of the sizing of the liposomes after drying (black, dark grey and grey curve) and in comparison to the size of liposomes before drying (light grey curve). Different film compositions were used.

FIG. 1a shows a schematic illustration of the drug delivery system 1 having a capsule device 2 with a first halve-capsule shell 2a and a second halve-capsule shell 2b being telescoped into each other thereby forming an aperture 3. The preparation 4 is shown in its compact form inside the capsule 2 with its one end 4a extending out of the aperture 3. An arrow indicated the direction of movement of the preparation 4 when the preparation 4 is pulled out of the capsule 2, i.e., the first halve-capsule shell 2a, through the aperture 3. In FIG. 1a, the aperture 3 is shown formed sidewise to a central axis A of the capsule 2 and arranged in the first halve-capsule shell 2a. In FIG. 2b, the aperture 3 is shown formed along the central axis A of the capsule 2 and arranged in the first-halve capsule shell 2a.

FIG. 2 shows a schematic illustration of a preparation 4 in its partially unfolded form. The preparation 4 is drawn having a sheet like shape. The central area of the preparation 4 is indicated in dashed lines, so that FIG. 2 essentially shows the end 4a of the preparation 4 protruding from the aperture 3 of the capsule device 2 and the still slightly coiled end 4b of the preparation 4. The coiled end 4b indicates the compact form of the preparation 4. At the end 4a, which extends through the aperture 3, a holding device 5 is shown having a patch like shape. The holding device 5 comprises a strip 5a. The strip 5a serves in the embodiment shown in FIG. 2 as connector to link the holding device 5 to the end 4a of the preparation 4. Alternatively, the end 4a of the preparation 4 is directly connected to a retainer, e.g., a string. In such an embodiment, the holding device 5, 5a is formed by the retainer itself.

FIG. 3 shows this preferred embodiment. The end 4a of the preparation 4 is shown having a sheet like shape, whereas a retainer 6 overlaps an end potion having a length d of the preparation 4 to form the holding device 5, 5a. The connection between the retainer 6 and the end 4a of the preparation 4 is made such that a pulling force can be transferred via the connection, when the retainer 6 is tensed to pull the preparation 4 out of the capsule device 2, e.g., by swallowing of the dosage form 1.

FIG. 4 shows the preparation 4 being spilt into several layers. In the shown embodiment of FIG. 4, the preparation is a wafer comprising three distinct layers 7. The one top layer 7a is formed as an adhesive layer, the central layer 7b contains the agent effective in the treatment or prevention of an esophageal disease, and the lower most drawn layer in FIG. 4 shows a protective, e.g., a water protective layer.

FIGS. 5a, 5b each show schematic illustrations of a capsule device 2 of the drug delivery system 1, with an aperture 3 formed by an overlapping wall part 9 or by telescoping two capsule halve-shells 2a, 2b into each other. As shown in FIG. 5a, the first halve-capsule shell 2a is slit over the second halve-capsule shell as indicated by the dashed lines. The second halve-capsule shell comprises a recess 8. By sliding the two halves 2a, 2b partially over each other, the first halve-capsule shell 2a covers the recess 8 of the second halve-capsule shell 2b partially. The further provided wall part 9, then covers the remaining open space formed by the recess 8 such that the aperture 3 is formed as an opening through which the preparation 4 can leave the shell 2.

Alternatively, FIG. 5b shows the embodiment, where the two halve-capsule shells 2a, 2b overlap in a joined position to such an extent that the aperture 3 is formed by the one, in particular, cylindrical wall of the first halve-capsule shell which overlaps the opening 10 of the second halve-capsule shell 2b.

FIG. 6 shows a schematic semi-transparent view of a drug delivery system 1. The first and second halve-capsule shells 2a, 2b are joined in a joined position, thereby form the aperture 3 by covering the opening 10 of the second halve-capsule shell 2b in this position. The end portion 4a of the preparation 4 is shown extending through the aperture 3. The pharmaceutical dosage form 1 further comprises a sinker element 11, which is located in the first halve-capsule shell 2a. The sinker extends from the first-halve capsule shell 2a into the second halve 2b, whereas notches 11a protrude from outside the capsule device 2 into the inside space to position the sinker 11 and to prevent the sinker from moving within the capsule. In FIG. 6 the preparation 4 is shown positioned underneath the sinker 11. The notches prevent the sinker 11 from sliding into the preparation 4.

FIG. 7 shows a schematic illustration of an applicator 12, with a holder 13 and a retainer 6 wound around the holder 13, whereas a drug delivery system 1 is positioned inside the holder 13. The applicator and the holder preferably have a cylindrical shape.

The capsule device 2 is positioned inside the holder 13 with the first halve 2a pointing towards applicator cap 12a. The cap 12a is removed for use. In the embodiment shown in FIG. 7, the capsule device 2 further comprises a sinker 11 located in the first halve-capsule shell 2a and the preparation 4 located in the second halve-capsule shell 2b. In the embodiment shown in FIG. 7, the first halve-capsule shell 2a is additionally pressed towards the bottom of the applicator 12. Therefore, a curved holder 13a is positioned above the capsule device 2. The holder 13a is curved such that its shape fits the shape of the first halve-capsule shell 2a. The pressing of the capsule 2 into the holder 13 is achieved by compression springs 14, whose one end is attached to the cap 12a of the applicator 12 and whose other end to the curved holder 13a. A drying element 15 is position inside the applicator 12 at the cap 12a of the applicator 12. This prevents the preparation 4 from being damaged by moisture. The applicator 12 does not necessarily comprise a curved holder 13, a drying element 15 or compression springs 14.

FIGS. 8a, 8b each show a schematic view of a patient taking the drug delivery system 1 using the applicator and drinking cup, before (FIG. 8a) and during (FIG. 8b) swallowing of the drug delivery system. FIG. 8a shows the administration of the drug delivery system comprising the capsule device 2 as herein described by a patient. A drinking cup 16 is filled with a liquid and an applicator 12 is attached to the cup 16. The applicator 12 comprises a retainer 6 and the drug delivery system 1, which further comprises the capsule device 2, connected to the preparation 4, and which is at least partially coiled at the inside of the capsule device 2. FIG. 8b illustrates the procedure when the patient swallows the dosage form 1 and the dosage form 1 then is transported through the esophagus towards the stomach. The retainer 12 pulls the preparation 4 out of the capsular device 2. The preparation 4 then spreads along the esophagus so that the active ingredient of the dosage form 1 is delivered to the mucosa of the esophagus.

### LIST OF REFERENCE SYMBOLS

1 drug delivery system
2 capsule device
2a first halve-capsule shell
2b second halve-capsule shell
3 aperture
4 preparation
4a end of the preparation that extends through the aperture
4b coiled end of the preparation
5 holding device
5a strip
6 retainer
7 layer
7a adhesive layer
7b active pharmaceutical ingredient—containing layer
7c protective layer
8 recess
9 wall part
10 opening
11 sinker element
11a notches
12 applicator
12a applicator cap
13 holder
13a curved holder
14 spring
15 drying element
16 drinking cap

### LIST OF ABBREVIATIONS

hPD-1.08 murine monoclonal anti-hPD-1 antibody
hPD-1.09 murine monoclonal anti-hPD-1 antibody
109A-H humanized IgGi 09A heavy chain sequence with zero back mutations
409A-H humanized IgG4 09A heavy chain sequence with zero antibody framework region back mutations
K09A-L-11 humanized 09A-kappa sequence with framework originally having CDR1 length of 11 aa
K09A-L-16 humanized 09A-kappa sequence with framework originally having CDR1 length of 16 aa
K09A-L-17 humanized 09A-kappa sequence with framework originally having CDR1 length of 17 aa

### EXAMPLES

### Example 1: Preparation of a Polymer Film with Paclitaxel

A “base polymer mixture” was prepared with the ingredients and the amount as depicted in Table 2 without addition of paclitaxel. The film was coated with paclitaxel after film preparation.

TABLE 2

| | Amount | | Proportion Solid |
|---|---|---|---|
| PVA Parteck ® | 18.0 | g | 90% |
| MXP (4-88) | | | |
| Glycerol | 2.0 | g | 10% |
| Brij ® L23-buffer | 80.0 | g | — |
| Coating | | | |
| Pacliaxel in MeOH | 10 | μg/mL | — |

The polymer film was prepared using solvent casting technology according to the following protocol:

### Solvent Casting Technology:

1. Preparation polymer mixture
2. Preparation of film laminates
3. Slitting of the moist laminates
4. Coating of the moist laminates
5. Drying at room temperature

The resulting films were flexible, air bubble free, optically homogenous and showed a uniform film surface.

Further, the film was analyzed for the content of paclitaxel. To this end, 5 circular samples with a size of 1 cm² were cut at random positions of the film and dissolved in Brij® L23-buffer/methanol (MeOH) for HPLC analysis with Hypersil ODS 150×4.6 mm as stationary phase and acetonitrile/phosphate buffer as mobile phase. The HPLC results are shown in FIG. 11 in comparison to a blank film sample and a calibration sample. Comparison of the results of the API-loaded film sample with the results of the calibration sample confirms that paclitaxel can be stably prepared in a film for use in the drug delivery device of the invention.

Further, the content of paclitaxel on the film was as indicated in Table 3.

TABLE 3

| | API per area |
|---|---|
| | (μg/cm²) |
| Mean value | 0.16 |
| SD* | 0.02 |
| Rel. SD** | 14% |

*Standard Deviation,
**relative Standard Deviation

The results in Table 3 show that loading of 1 cm² of the film with about 0.2 pg is possible. It must be noted that standard deviation and maximum of drug load are determined by the process technology and do not represent any limitations in principle.

Paclitaxel is insoluble in water and poorly absorbable. Nevertheless, the inventors could surprisingly show stable loading of paclitaxel onto a film which can be used in the drug delivery system of the invention. Further, chemotherapeutic, parenteral administration of paclitaxel in the art, requires high doses of 220 mg/m² with a three week break and is associated with side effects, such alopecia, nausea, vomiting, diarrhea, blood count change, neuropathy, and myalgia. Topical and/or local administration of paclitaxel via the drug delivery system of the invention allows for improved local administration to the target site thereby increasing the value of this drug, particularly in the treatment of esophageal diseases.

### Example 2: Preparation of a Polymer Film with RNA

For testing of the stability of RNA as active pharmaceutical ingredient in the drug delivery system of the invention, the following sequence was synthesized:

| (SEQ ID NO: 25) | |
|---|---|
| | 5′ UGC GCA GAA UGA GAU GAG UUG 3′ |

A “base polymer mixture” was prepared with the ingredients and the amount as depicted in Table 4 without addition of paclitaxel.

TABLE 4

| | Proportion Total |
|---|---|
| PVA 18-88 | 18% |
| Glycerol | 2% |
| Dem. Water | 80% |

The polymer film was prepared using solvent casting technology according to the following protocol:

### Solvent Casting Technology

1. preparation “base polymer mixture”
2. sterilization
3. preparation of film laminates
4. drying
5. cutting (A=0.25 cm²)
6. application of RNA samples on the polymer film
7. drying of the final samples
   !points 3-7 under aseptic conditions!

Stability of RNA on the film was analyzed using a 20% denaturizing polyacrylamide (PAA) gel. The film has no influence on the stability of the RNA as shown in FIG. 12. The experiment was repeated with a commercially available siRNA. The inventors observed a comparable stability with a commercial siRNA targeting IL-6 (Thermo Fisher; FIG. 13)

Stability of RNA on the film was further analyzed before and after solubilization of the film. Storage of the dried samples on the film had no influence on RNA stability as shown in FIG. 14 demonstrating that the drug delivery device including an RNA as active pharmaceutical ingredient is storage stable. Storage after solubilization of the film showed a slight degradation of RNA in the solvent which could be reversed by the addition of RNAse inhibitor (FIG. 15).

The results demonstrate that RNA remains stable after coating to the film and also under various storage conditions and can thus be used as active pharmaceutical ingredient with the drug delivery system thereby providing novel treatment options for the drug delivery system, particularly in the treatment of esophageal diseases.

### Example 3: Analysis of the Stability of Antisense RNA for BMP2 on the Film Under Various Application Conditions and Storage Conditions

For testing of the stability of antisense RNA for BMP2 as active pharmaceutical ingredient in the drug delivery system of the invention, the following sequence was synthesized:

5′-AUU UCG AGU UGG CUG UUG C-3′ (SEQ ID NO:23) wherein an overhang consisting of two UU (uridine nucleotides) are attached to the 3′end.

This sequence was used in unlabeled form and in labeled form (labeled with the dye Atto633 which was attached to the 3′ end to visualize the ribonucleic acid during preparation (FIG. 10)) for the stability experiments.

A film containing the antisense RNA for BMP2 was prepared as described in Example 2 except for using PVA 4-88 instead of PVA 18-88.

The stability of the antisense BMP2 RNA was analyzed under various application conditions as indicated in FIG. 16 and the legend of FIG. 16. 10 pmol of each probe was loaded on a 20% denaturing polyacrylamide gel. The gel was run for 3 hours at 140 volt and subsequently colored with SYBR gold for 20 minutes.

The stability of labeled antisense BMP2 RNA was analyzed in comparison to unlabeled antisense BMP2 RNA under various application conditions as indicated in FIG. 17 and the legend of FIG. 17. In lanes 1 to 5, 5 pmol of the probe, in lanes 4 to 6, 10 pmol of each probe were loaded on a 20% denaturing polyacrylamide gel. The gel was run for 3 hours at 140 volt and subsequently colored with ethidium bromide for 30 minutes (FIG. 17a) or excited with light of a wavelength of 630 nm and emission was measured at 675 nm (FIG. 17b). The bands of labeled BMP2 RNA in FIG. 17b correspond to the band of the unlabeled BMP2 RNA in FIG. 17a Since the unlabeled BMP2 RNA has no fluorescent marker, the bands of unlabeled BMP2 RNA are not visible in FIG. 17b.

The stability of labeled antisense BMP2 RNA was analyzed in comparison to unlabeled antisense BMP2 RNA after storage of the dry film for 10 days at 4° C. Probes were loaded on a 20% denaturing polyacrylamide gel. The gel was run for 3 hours at 140 volt and subsequently colored with ethidium bromide for 30 minutes (FIG. 18b) or excited with light of a wavelength of 630 nm and emission was measured at 675 nm (FIG. 18a). The bands of labeled BMP2 RNA in FIG. 18a correspond to the band of the unlabeled BMP2 RNA in FIG. 18b.

The results can be summarized as follows:

After application of BMP2 RNA to the film, no difference between the test sample and the stock sample can be observed in the each of the individual experiments. This is also true for labeled samples with increased sensitivity.

BMP2 RNA is not degraded by the film.

Slight bending of the bands in the gel can be explained by the presence of the polymer in the gel.

The polymer did not negatively impact the stability of the RNA at each of the experimentally tested time points.

Even after 10 days of storage of the dry film at 4° C., no RNA degradation could be observed.

The above results demonstrate that the drug delivery system of the invention is suitable for administration of RNA which opens novel treatment options for the drug delivery system.

### Example 4: Encapsulation of RNA into Liposomes

### Liposome Preparation

Liposome preparation was performed according to the thin layer hydration (TLH) method, which is based on a protocol by Bangham [1]. To this end, a chloroform solution containing an appropriate amount of 1,2-dioleoyl-sn-glycero-3-phosphocholine (DOPC) was placed in a glass tube and dried under nitrogen flow. For comprehensive drying, the samples were incubated overnight under vacuum.

The dried lipid film was dissolved in 1 mL Tris-HCl (pH 8) containing 250 mM sucrose (1.66 mg/mL).

Next, the vesicles were frozen eight times in liquid nitrogen and thawed at 54° C. The extrusion method with a 100 nm polycarbonate membrane was used for homogenization.

### Liposomal Encapsulation of RNA

For the experiments, the RNA sequence 5′-AUU UCG AGU UGG CUG UUG CUU-3′ (SEQ ID NO:23) was used because it was part of an siRNA which was declared as an efficient siRNA for BMP-2 in Wu et al. [2]. The RNA was purchased for this work from biomers.net GmbH in HPLC pure grade.

For encapsulation, 7.5 nmol RNA was used [3] and redissolved with 150 μL of the liposome solution afterwards. To the mixture of liposomes and RNA, 100 μL of ethanol containing 10 mM CaCl₂ was slowly instilled at room temperature with constant mixing. Subsequently, the samples were dialyzed against liposome buffer at 4° C. overnight.

### Purification of Liposomal Encapsulated RNA

Magnetic particles were used to separate the free RNA from the liposomal encapsulated RNA. These particles are coated by a silica layer and have quaternary aminoethyl groups on the surface [4]. Due to the resulting positive charges on the surface, the free RNA is bound via the negative backbone [4] and can thus be separated from the liposomal encapsulated RNA.

The protocol was inspired by the publication of Ye and Beverly [4]. All separation was performed at room temperature. For one sample of liposomes, 20 μL, and 5 μL each of resuspended magnetic particles were added to a reaction tube in the first step. The supernatant was removed from these after 2 min on a magnetic separator. For purification and equilibration of the particles, 200 μL of liposome buffer was added to the particles and incubated for 2 min. After 2 min of magnetic separation, the supernatant could be removed. Subsequently, the liposomes were added to the reaction vessel containing 20 μL of magnetic particle suspension and incubated for 30 s after resuspension. After 2 min of separation, the liposome sample could be removed. The procedure was repeated with the reaction tubes containing 5 μL of equilibrated magnetic particle suspension.

### Size Determination

The dynamic light scattering (DSL) method was used to determine the size of the liposomes. Prior to use on the drug delivery system, 100 μL of the 1:100 diluted sample was placed in a microcuvette with a 10 mm passage length. The measurement took place at 25° C. after a two-minute equilibration period. Water was used as the standard (refractive index 1.33; absorbance 0.001; viscosity 0.833).

### Encapsulation Efficiency

The encapsulation efficiency (EE) can be calculated if the RNA concentration in the liposomes after purification is determined and this is put into a ratio with the amount of substance used (formula 1).

$$E E \% = \frac{n ( end )}{n ( start )} \times 100 \qquad ( 1 )$$

n(end): Amount of substance after purification of liposomes from free RNA.

n(start): Amount of substance used for liposome preparation.

Quant-it™ RiboGreen was used to determine the concentration of RNA. The RNA that was used for encapsulation was also used to prepare the standard series.

For this experiment, a TE buffer consisting of 10 mM Tris-HCl and 1 mM EDTA with a pH of 7.5 was used when only the RNA outside the liposomes was to be determined. When total RNA was determined, 2% TritonX-100 was added to the TE buffer.

The liposomes for the standard series were diluted 1:500. The diluted liposome sample and the stock RNA solution (1 μmol/L) were used to prepare the standard series (0, 6, 30, 50, 100, 150 nmol/L), each with a volume of 100 μL.

The samples were also diluted 1:500. Subsequently, 100 μL of the 1:200 diluted dye was added to each standard, or sample. After brief mixing, the samples were incubated under protection from light for 3 min. Measurement of fluorescence took place at an excitation wavelength of approximately 480 nm. Emission was measured at 525 nm. For the determination of encapsulated RNA, the difference between the amount of substance with and without TritonX-100 was taken. This result was used for the calculation of EE (formula 2 below).

### Application on the Drug Delivery Device

For the application of liposomal encapsulated RNA, only the film in an analytically necessary size of approximately 1 cm² was used with and without the addition of sucrose.

TABLE 5
Composition of polymer mixture for film preparation
by solvent casting technology.

| Polymer blend | 1. without addition | 2. with addition |
|---|---|---|
| PVA 18-88 | 18.0% | 18.0% |
| Glycerol (anhydrous) | 2.0% | 2.0% |
| Water | 80.0% | 71.4% |
| Sucrose | — | 8.6% |

For drying, approximately 40 pmol of liposomal encapsulated RNA was applied to the film. The film was dried for 3 h in a desiccator under vacuum. After drying, the films were stored at 4° C. in a refrigerator.

For analysis of the dried liposomes, the films were dissolved with 100 μL of deionized water each. Subsequently, the retention of RNA and the size of liposomes were determined.

For measurement by DLS, 100 μL of the dissolved film diluted 1:10 was used in the microcuvette. Water was used as standard, only the viscosity had to be adjusted, which was determined to be 2.4 mPa*s using a rotational rheometer.

For the determination of retention, the concentration of RNA was determined using Quanti-it™ RiboGreen reagent without TritonX-100 in the sample. In this case, the dissolved film was diluted 1:84.

Since small amounts of RNA are still present outside the liposomes after separation, this must be subtracted from the determined amount of RNA. Subsequently, the retention can be calculated according to formula 2. Here, n(outside) is the determined amount of RNA substance outside the liposomes after drying, subtracting the RNA that was already outside the liposomes before drying, and n(inside, sol) is the amount of RNA substance that was determined to be encapsulated before drying.

$$Retention [ \% ] = ( 1 - \frac{{n}_{( outside )}}{{n}_{( inside , sol )}} ) \times 100 \qquad ( 2 )$$

TABLE 6
Values on average size, PDI and EE

| Size | Value |
|---|---|
| Average size according to | 191.7 nm |
| the area of the maximum | |
| PDI | 0.2075 |
| EE | 10.72% |

### Results

After the preparation steps to encapsulate RNA, the average size of liposomes was 191.7 nm (Table 6). For the same batch, an EE of 10.72% could be determined, thus 0.804 nmol were encapsulated. With simultaneous encapsulation of 0.804 nmol, there were still 120 pmol outside the liposomes. This had to be taken into account when determining retention. Subsequent drying was performed in three different ways, which were subsequently compared for change in size of the liposomes and retention of RNA. FIG. 20a shows the graphs of the size determination of the liposomes after drying and rehydration. The curve of liposomes before drying was inserted for comparison in light grey. The curve of liposomes dried on a film to which no sucrose was added is shown in grey. After rehydration, the average size of liposomes after drying was slightly smaller (187 nm) than before drying (FIG. 20a). Both the black curve sample and dark grey curve sample were dried on a film with sucrose. In addition, liposome buffer was added to the sample of the black curve as a protection buffer in the same volume fraction before it was applied to the film for drying. A clear reduction in the diameter of the liposomes can be measured for both samples (FIG. 20a).

For the retention of RNA after drying the liposomes, a value above 95% was achieved for all samples. This is a better retention than the control, which was slightly higher at 90.255% than previously published by Wolkers et al [5]. For the sample dried on a film without sucrose, no release of RNA could be measured. The other two samples, which were dried on the film with sucrose, also showed very good retentions of 95.13% and 96.13% (with protection buffer).

The retention in all experiments is above the control experiment and the liposomes all remain approximately in the size range which is recommended at least for systemic therapy [3].

### Example 5: Preparation of a Polymer Film by Melt Extrusion

Selecting the appropriate polymer base: choose a polymer base that is compatible with the active ingredients you intend to use. Typical polymers are polyvinyl alcohol (PVA), cellulose ether and polyethylene glycol (PEG) in combination with suitable plasticizers.
Active ingredient preparation: The active ingredient is usually prepared in a suitable formulation (e.g., as powder or granules).
Mixing of the components: The polymer base and the active ingredient are mixed in a mixer to form a homogeneous mixture.
Extrusion: The mixture is fed into an extruder, where it is melted at high temperatures and forced through a die. The die forms the film, which is deposited on a cooling plate.
After the polymer compound is forced through the die during melt extrusion, the extruded film can undergo a rolling process to further optimize its thickness and properties. The rolling process can include either cold or hot rolling, depending on the specific requirements of the film.
Post-treatment: After the extrusion process, the extruded film is cut to the desired size and shape. The film is then usually subjected to further processes such as drying, coating or laminating to improve its physical and pharmaceutical properties.

### LIST OF REFERENCES

1 Trucillo P, Campardelli R, Reverchon E. Liposomes: From Bangham to Supercritical Fluids. Processes. Aug. 21, 2020; 8(9):1022.
2. Wu J B, Fu H Q, Huang L Z, Liu A W, Zhang J X. Effects of siRNA-targeting BMP-2 on the abilities of migration and invasion of human liver cancer SMMC7721 cells and its mechanism. Cancer Gene Ther. January 2011; 18(1):20-5.
3. Somiya M, Yamaguchi K, Liu Q, Niimi T, Maturana A D, lijima M, et al. One-step scalable preparation method for non-cationic liposomes with high siRNA content. International Journal of Pharmaceutics. July 2015; 490(1-2):316-23.
4. Ye G, Beverly M. The use of strong anion-exchange (SAX) magnetic particles for the extraction of therapeutic siRNA and their analysis by liquid chromatography/mass spectrometry. Rapid Communications in Mass Spectrometry. 2011; 25(21):3207-15.
5. Wolkers W F, Oldenhof H, Tablin F, Crowe J H. Preservation of dried liposomes in the presence of sugar and phosphate. Biochimica et Biophysica Acta (BBA)—Biomembranes. March 2004; 1661(2):125-34.

List of Sequences:
SEQ ID NO: 1:
Lys His His Ser Gln Arg Ala Arg Lys Lys Asn Lys
Asn Cys Arg Arg His Ser Leu Tyr Val Asp Phe Ser
Asp Val Gly Trp Asn Asp Trp Ile Val Ala Pro Pro
Gly Tyr Gln Ala Phe Tyr Cys His Gly Asp Cys Pro
Phe Pro Leu Ala Asp His Leu Asn Ser Thr Asn His
Ala Ile Val Gln Thr Leu Val Asn Ser Val Asn Ser
Ser Ile Pro Lys Ala Cys Cys Val Pro Thr Glu Leu
Ser Ala Ile Ser Met Leu Tyr Leu Asp Glu Tyr Asp
Lys Val Val Leu Lys Asn Tyr Gln Glu Met Val Val
Glu Gly Cys Gly Cys Arg
SEQ ID NO: 2:
Gly Arg Thr Phe Ser Thr Tyr Ala
SEQ ID NO: 3:
Ser Lys Gly Gly Gly Ile Thr Tyr
SEQ ID NO: 4:
Asp Pro Val Ser Ser Val Ala Lys Ser Pro Val Ala
Tyr Pro
SEQ ID NO: 5:
Gly Arg Thr Phe Arg Ile Asn Asp
SEQ ID NO: 6:
Thr Ser Gly Gly Asn Thr Asn
SEQ ID NO: 7:
Asp Gly Leu Arg Phe Asp Ser Thr Arg Tyr Arg Pro
Phe Asp
SEQ ID NO: 8:
Gly Ser Ile Arg Gly Phe Val Ala
SEQ ID NO: 9:
Thr Asn Gly Gly Thr Leu
SEQ ID NO: 10:
Arg Gln Ile Gly Ala Ser Gly Tyr Asp
SEQ ID NO: 11:
Glu Val Gln Leu Val Glu Ser Gly Gly Gly Leu Val
Gln Ala Gly Gly Ser Leu Arg Leu Ser Cys Ala Ala
Ser Gly Arg Thr Phe Ser Thr Tyr Ala Met Gly Trp
Phe Arg Gln Ala Pro Gly Lys Glu Arg Glu Phe Val
Ala Ala Ile Ser Lys Gly Gly Gly Ile Thr Tyr Tyr
Ser Asp Ser Val Arg Gly Arg Phe Thr Ile Ser Lys
Glu Asn Ala Lys Asn Thr Val Tyr Leu Gln Met Asn
Ser Leu Lys Pro Glu Asp Thr Ala Val Tyr Tyr Cys
Ala Ala Asp Pro Val Ser Ser Val Ala Lys Ser Pro
Val Ala Tyr Pro Tyr Trp Gly Gln Gly Thr Gln Val
Thr Val Ser Ser
SEQ ID NO: 12:
Glu Val Gln Leu Val Glu Ser Gly Gly Gly Leu Val
Gln Ala Gly Gly Ser Leu Arg Leu Ser Cys Ala Ala
Ser Gly Arg Thr Phe Arg Ile Asn Asp Met Gly Trp
Tyr Arg Gln Ala Pro Gly Lys Ser Arg Asp Met Val
Ala Arg Ile Thr Ser Gly Gly Asn Thr Asn Tyr Ala
Asp Ser Val Lys Gly Arg Phe Thr Ile Ser Arg Asp
Asn Ala Lys Asn Thr Val Tyr Leu Gln Met Asn Ser
Leu Lys Pro Glu Asp Thr Ala Val Tyr Tyr Cys Asn
Ala Asp Gly Leu Arg Phe Asp Ser Thr Arg Tyr Arg
Pro Phe Asp Ser Trp Gly Gln Gly Thr Gln Val Thr
Val Ser Ser
SEQ ID NO: 13:
Glu Val Gln Leu Val Glu Ser Gly Gly Gly Leu Val
Gln Ala Gly Gly Ser Leu Arg Leu Ser Cys Ala Ala
Ser Gly Ser Ile Arg Gly Phe Val Ala Met Ala Trp
Tyr Arg Gln Ala Pro Gly Lys Gln Arg Glu Trp Val
Ala Thr Val Thr Asn Gly Gly Thr Leu Tyr Gly Asp
Ser Val Lys Gly Arg Phe Thr Gly Ser Arg Asp Asn
Ala Lys Asn Thr Val Tyr Leu Gln Met Asn Ser Leu
Lys Pro Glu Asp Thr Ala Val Tyr Tyr Cys Ala Leu
Arg Gln Ile Gly Ala Ser Gly Tyr Asp Tyr Trp Gly
Gln Gly Thr Gln Val Thr Val Ser Ser
SEQ ID NO: 14:
Gln Ala Lys His Lys Gln Arg Lys Arg Leu Lys Ser
Ser Cys Lys Arg His Pro Leu Tyr Val Asp Phe Ser
Asp Val Gly Trp Asn Asp Trp Ile Val Ala Pro Pro
Gly Tyr His Ala Phe Tyr Cys His Gly Glu Cys Pro
Phe Pro Leu Ala Asp His Leu Asn Ser Thr Asn His
Ala Ile Val Gln Thr Leu Val Asn Ser Val Asn Ser
Lys Ile Pro Lys Ala Cys Cys Val Pro Thr Glu Leu
Ser Ala Ile Ser Met Leu Tyr Leu Asp Glu Asn Glu
Lys Val Val Leu Lys Asn Tyr Gln Asp Met Val Val
Glu Gly Cys Gly Cys Arg
SEQ ID NO: 15:
Met Val Ala Gly Thr Arg Cys Leu Leu Ala Leu Leu
Leu Pro Gln Val Leu Leu Gly Gly Ala Ala Gly Leu
Val Pro Glu Leu Gly Arg Arg Lys Phe Ala Ala Ala
Ser Ser Gly Arg Pro Ser Ser Gln Pro Ser Asp Glu
Val Leu Ser Glu Phe Glu Leu Arg Leu Leu Ser Met
Phe Gly Leu Lys Gln Arg Pro Thr Pro Ser Arg Asp
Ala Val Val Pro Pro Tyr Met Leu Asp Leu Tyr Arg
Arg His Ser Gly Gln Pro Gly Ser Pro Ala Pro Asp
His Arg Leu Glu Arg Ala Ala Ser Arg Ala Asn Thr
Val Arg Ser Phe His His Glu Glu Ser Leu Glu Glu
Leu Pro Glu Thr Ser Gly Lys Thr Thr Arg Arg Phe
Phe Phe Asn Leu Ser Ser Ile Pro Thr Glu Glu Phe
Ile Thr Ser Ala Glu Leu Gln Val Phe Arg Glu Gln
Met Gln Asp Ala Leu Gly Asn Asn Ser Ser Phe His
His Arg Ile Asn Ile Tyr Glu Ile Ile Lys Pro Ala
Thr Ala Asn Ser Lys Phe Pro Val Thr Arg Leu Leu
Asp Thr Arg Leu Val Asn Gln Asn Ala Ser Arg Trp
Glu Ser Phe Asp Val Thr Pro Ala Val Met Arg Trp
Thr Ala Gln Gly His Ala Asn His Gly Phe Val Val
Glu Val Ala His Leu Glu Glu Lys Gln Gly Val Ser
Lys Arg His Val Arg Ile Ser Arg Ser Leu His Gln
Asp Glu His Ser Trp Ser Gln Ile Arg Pro Leu Leu
Val Thr Phe Gly His Asp Gly Lys Gly His Pro Leu
His Lys Arg Glu Lys Arg Gln Ala Lys His Lys Gln
Arg Lys Arg Leu Lys Ser Ser Cys Lys Arg His Pro
Leu Tyr Val Asp Phe Ser Asp Val Gly Trp Asn Asp
Trp Ile Val Ala Pro Pro Gly Tyr His Ala Phe Tyr
Cys His Gly Glu Cys Pro Phe Pro Leu Ala Asp His
Leu Asn Ser Thr Asn His Ala Ile Val Gln Thr Leu
Val Asn Ser Val Asn Ser Lys Ile Pro Lys Ala Cys
Cys Val Pro Thr Glu Leu Ser Ala Ile Ser Met Leu
Tyr Leu Asp Glu Asn Glu Lys Val Val Leu Lys Asn
Tyr Gln Asp Met Val Val Glu Gly Cys Gly Cys Arg
SEQ ID NO: 16:
Met Ile Pro Gly Asn Arg Met Leu Met Val Val Leu
Leu Cys Gln Val Leu Leu Gly Gly Ala Ser His Ala
Ser Leu Ile Pro Glu Thr Gly Lys Lys Lys Val Ala
Glu Ile Gln Gly His Ala Gly Gly Arg Arg Ser Gly
Gln Ser His Glu Leu Leu Arg Asp Phe Glu Ala Thr
Leu Leu Gln Met Phe Gly Leu Arg Arg Arg Pro Gln
Pro Ser Lys Ser Ala Val Ile Pro Asp Tyr Met Arg
Asp Leu Tyr Arg Leu Gln Ser Gly Glu Glu Glu Glu
Glu Gln Ile His Ser Thr Gly Leu Glu Tyr Pro Glu
Arg Pro Ala Ser Arg Ala Asn Thr Val Arg Ser Phe
His His Glu Glu His Leu Glu Asn Ile Pro Gly Thr
Ser Glu Asn Ser Ala Phe Arg Phe Leu Phe Asn Leu
Ser Ser Ile Pro Glu Asn Glu Val Ile Ser Ser Ala
Glu Leu Arg Leu Phe Arg Glu Gln Val Asp Gln Gly
Pro Asp Trp Glu Arg Gly Phe His Arg Ile Asn Ile
Tyr Glu Val Met Lys Pro Pro Ala Glu Val Val Pro
Gly His Leu Ile Thr Arg Leu Leu Asp Thr Arg Leu
Val His His Asn Val Thr Arg Trp Glu Thr Phe Asp
Val Ser Pro Ala Val Leu Arg Trp Thr Arg Glu Lys
Gln Pro Asn Tyr Gly Leu Ala Ile Glu Val Thr His
Leu His Gln Thr Arg Thr His Gln Gly Gln His Val
Arg Ile Ser Arg Ser Leu Pro Gln Gly Ser Gly Asn
Trp Ala Gln Leu Arg Pro Leu Leu Val Thr Phe Gly
His Asp Gly Arg Gly His Ala Leu Thr Arg Arg Arg
Arg Ala Lys Arg Ser Pro Lys His His Ser Gln Arg
Ala Arg Lys Lys Asn Lys Asn Cys Arg Arg His Ser
Leu Tyr Val Asp Phe Ser Asp Val Gly Trp Asn Asp
Trp Ile Val Ala Pro Pro Gly Tyr Gln Ala Phe Tyr
Cys His Gly Asp Cys Pro Phe Pro Leu Ala Asp His
Leu Asn Ser Thr Asn His Ala Ile Val Gln Thr Leu
Val Asn Ser Val Asn Ser Ser Ile Pro Lys Ala Cys
Cys Val Pro Thr Glu Leu Ser Ala Ile Ser Met Leu
Tyr Leu Asp Glu Tyr Asp Lys Val Val Leu Lys Asn
Tyr Gln Glu Met Val Val Glu Gly Cys Gly Cys Arg
SEQ ID NO: 17:
gcaggucuuu gcaccaaga
SEQ ID NO: 18:
gcaacagcca acucgaaau
SEQ ID NO: 19:
gcuguaccuu gacgagaau
SEQ ID NO: 20:

| | |
|---|---|
| gccgccgccg ccgucgccgc cgccggaguc cucgccccgc | 60 |
| cgcgcugcgc ccggcucgcg | |
| cugcgcuagu cgcuccgcuu cccacacccc gccggggacu | 120 |
| ggcagccgcc gccgcacauc | |
| ugccgccaca gccuccgccg gcuacccgaa cguucucggg | 180 |
| gccagcgccg aguggaucac | |
| cggggaccgc gaggcacccg cgcgccgcag accccgcgcg | 240 |
| ggcuggagca cccggcagag | |
| cgcgccacag cgccguggcc ucugcugccc gggcugcgcc | 300 |
| agagccgcgg acgggcgcgc | |
| agagcgccgg ggacuccgga gccgaucccu agcgccgcga | 360 |
| ugcggagcac cuacugcagg | |
| agaucggggg ccugggacgc gcuggccgag gugugaucgg | 420 |
| accccaggcu agccacaaag | |
| ggcacuuggc cccagggcua ggagagcgag gggagagcac | 480 |
| agccacccgo cucggcggcc | |
| cgggacucgg cucgacucgc cggagaaugc gcccgaggac | 540 |
| gacggggcgc cagagccgcg | |
| gugcuuucaa cuggcgagcg cgaauggggg ugcacuggag | 600 |
| uaaggcagag ugaugcgggg | |
| gggcaacucg ccuggcaccg agaucgccgc cgugcccuuc | 660 |
| ccuggacccg gcgucgccca | |
| ggauggcugc cccgagccau gggccgcggc ggagcuagcg | 720 |
| cggagcgccc gacccucgac | |
| ccccgagucc cggagccggc cccgcgcggg gccacgcguc | 780 |
| ccucgggcgc ugguuccuaa | |
| ggaggacgac agcaccagcu ucuccuuucu cccuucccuu | 840 |
| cccugccccg cacuccuccc | |
| ccugcucgcu guuguugugu gucagcacuu ggcuggggac | 900 |
| uucuugaacu ugcagggaga | |
| auaacuugcg caccccacuu ugcgccggug ccuuugcccc | 960 |
| agcggagccu gcuucgccau | |
| cuccgagccc caccgccccu ccacuccucg gccuugcccg | 1020 |
| acacugagac gcuguuccca | |
| gcgugaaaag agagacugcg cggccggcac ccgggagaag | 1080 |
| gaggaggcaa agaaaaggaa | |
| cggacauucg guccuugcgc cagguccuuu gaccagaguu | 1140 |
| uuuccaugug gacgcucuuu | |
| caauggacgu guccccgcgu gcuucuuaga cggacugcgg | 1200 |
| ucuccuaaag gucgaccaug | |
| guggccggga cccgcugucu ucuagcguug cugcuucccc | 1260 |
| agguccuccu gggcggcgcg | |
| gcuggccucg uuccggagcu gggccgcagg aaguucgcgg | 1320 |
| cggcgucguc gggccgcccc | |
| ucaucccagc ccucugacga gguccugagc gaguucgagu | 1380 |
| ugcggcugcu cagcauguuc | |
| ggccugaaac agagacccac ccccagcagg gacgccgugg | 1440 |
| ugccccccua caugcuagac | |
| cuguaucgca ggcacucagg ucagccgggc ucacccgccc | 1500 |
| cagaccaccg guuggagagg | |
| gcagccagcc gagccaacac ugugcgcagc uuccaccaug | 1560 |
| aagaaucuuu ggaagaacua | |
| ccagaaacga gugggaaaac aacccggaga uucuucuuua | 1620 |
| auuuaaguuc uauccccacg | |
| gaggaguuua ucaccucagc agagcuucag guuuuccgag | 1680 |
| aacagaugca agaugcuuua | |
| ggaaacaaua gcaguuucca ucaccgaauu aauauuuaug | 1740 |
| aaaucauaaa accugcaaca | |
| gccaacucga aauuccccgu gaccagacuu uuggacacca | 1800 |
| gguuggugaa ucagaaugca | |
| agcagguggg aaaguuuuga ugucaccccc gcugugaugc | 1860 |
| gguggacugc acagggacac | |
| gccaaccaug gauucguggu ggaaguggcc cacuuggagg | 1920 |
| agaaacaagg ugucuccaag | |
| agacauguua ggauaagcag gucuuugcac caagaugaac | 1980 |
| acagcugguc acagauaagg | |
| ccauugcuag uaacuuuugg ccaugaugga aaagggcauc | 2040 |
| cucuccacaa aagagaaaaa | |
| cgucaagcca aacacaaaca gcggaaacgc cuuaagucca | 2100 |
| gcuguaagag acacccuuug | |
| uacguggacu ucagugacgu gggguggaau gacuggauug | 2160 |
| uggcuccccc gggguaucac | |
| gccuuuuacu gccacggaga augcccuuuu ccucuggcug | 2220 |
| aucaucugaa cuccacuaau | |
| caugccauug uucagacguu ggucaacucu guuaacucua | 2280 |
| agauuccuaa ggcaugcugu | |
| gucccgacag aacucagugc uaucucgaug cuguaccuug | 2340 |
| acgagaauga aaagguugua | |
| uuaaagaacu aucaggacau gguuguggag gguugugggu | 2400 |
| gucgcuagua cagcaaaauu | |
| aaauacauaa auauauauau auauauauau uuuagaaaaa | 2460 |
| agaaaaaaac aaacaaacaa | |
| aaaaacccca ccccaguuga cacuuuaaua uuucccaaug | 2520 |
| aagacuuuau uuauggaaug | |
| gaauggaaaa aaaaacagcu auuuugaaaa uauauuuaua | 2580 |
| ucuacgaaaa gaaguuggga | |
| aaacaaauau uuuaaucaga gaauuauucc uuaaagauuu | 2640 |
| aaaauguauu uaguuguaca | |
| uuuuauaugg guucaacccc agcacaugaa guauaauggu | 2700 |
| cagauuuauu uuguauuuau | |
| uuacuauuau aaccacuuuu uaggaaaaaa auagcuaauu | 2760 |
| uguauuuaua uguaaucaaa | |
| agaaguaucg gguuuguaca uaauuuucca aaaauuguag | 2820 |
| uuguuuucag uuguguguau | |
| uuaagaugaa aagucuacau ggaagguuac ucuggcaaag | 2880 |
| ugcuuagcac guuugcuuuu | |
| uugcagugcu acuguugagu ucacaaguuc aaguccagaa | 2940 |
| aaaaaaagug gauaauccac | |
| ucugcugacu uucaagauua uuauauuauu caauucucag | 3000 |
| gaauguugca gagugauugu | |
| ccaauccaug agaauuuaca uccuuauuag guggaauauu | 3060 |
| uggauaagaa ccagacauug | |
| cugaucuauu auagaaacuc uccuccugcc ccuuaauuua | 3120 |
| cagaaagaau aaagcaggau | |
| ccauagaaau aauuaggaaa acgaugaacc ugcaggaaag | 3180 |
| ugaaugaugg uuuguuguuc | |
| uucuuuccua aauuagugau cocuucaaag gggcugaucu | 3240 |
| ggccaaagua uucaauaaaa | |
| cguaagauuu cuucauuauu gauauugugg ucauauauau | 3300 |
| uuaaaauuga uaucucgugg | |
| cccucaucaa ggguuggaaa uuuauuugug uuuuaccuuu | 3360 |
| accucaucug agagcucuuu | |
| auucuccaaa gaacccaguu uucuaacuuu uugcccaaca | 3420 |
| cgcagcaaaa uuaugcacau | |
| cguguuuucu gcccacccuc uguucucuga ccuaucagcu | 3480 |
| ugcuuuucuu uccaagguug | |
| uguguuugaa cacauuucuc caaauguuaa accuauuuca | 3540 |
| gauaauaaau aucaaaucuc | |
| uggca | 3545 |
| SEQ ID NO: 21: | |
| agacgcagac gcagaggucg agcgcaggcc gaaagcuguu | 60 |
| caccguuuuc ucgacuccgg | |
| ggaacaugga gccauuccgu agugccaucc cgagcaacgc | 120 |
| acugcugcag cuucccugag | |
| ccuuuccagc aaguuuguuc aagauuggcu gucaagaauc | 180 |
| auggacuguu auuauaugcc | |
| uuguuuucug ucaagacacc augauuccug guaaccgaau | 240 |
| gcugaugguc guuuuauuau | |
| gccaaguccu gcuaggaggc gcgagccaug cuaguuugau | 300 |
| accugagacg gggaagaaaa | |
| aagucgccga gauucagggc cacgcgggag gacgccgcuc | 360 |
| agggcagagc caugagcucc | |
| ugcgggacuu cgaggcgaca cuucugcaga uguuugggcu | 420 |
| gcgccgccgc ccgcagccua | |
| gcaagagugc cgucauuccg gacuacaugc gggaucuuua | 480 |
| ccggcuucag ucuggggagg | |
| aggaggaaga gcagauccac agcacugguc uugaguaucc | 540 |
| ugagcgcccg gccagccggg | |
| ccaacaccgu gaggagcuuc caccacgaag aacaucugga | 600 |
| gaacauccca gggaccagug | |
| aaaacucugc uuuucguuuc cucuuuaacc ucagcagcau | 660 |
| cccugagaac gaggugaucu | |
| ccucugcaga gcuucggcuc uuccgggagc agguggacca | 720 |
| gggcccugau ugggaaaggg | |
| gcuuccaccg uauaaacauu uaugagguua ugaagccccc | 780 |
| agcagaagug gugccugggc | |
| accucaucac acgacuacug gacacgagac ugguccacca | 840 |
| caaugugaca cggugggaaa | |
| cuuuugaugu gagcccugcg guccuucgcu ggacccggga | 900 |
| gaagcagcca aacuaugggc | |
| uagccauuga ggugacucac cuccaucaga cucggaccca | 960 |
| ccagggccag caugucagga | |
| uuagccgauc guuaccucaa gggaguggga auugggccca | 1020 |
| gcuccggccc cuccugguca | |
| ccuuuggcca ugauggccgg ggccaugccu ugacccgacg | 1080 |
| ccggagggcc aagcguagcc | |
| cuaagcauca cucacagcgg gccaggaaga agaauaagaa | 1140 |
| cugccggcgc cacucgcucu | |
| auguggacuu cagcgaugug ggcuggaaug acuggauugu | 1200 |
| ggccccacca ggcuaccagg | |
| ccuucuacug ccauggggac ugccccuuuc cacuggcuga | 1260 |
| ccaccucaac ucaaccaacc | |
| augccauugu gcagacccug gucaauucug ucaauuccag | 1320 |
| uauccccaaa gccuguugug | |
| ugcccacuga acugagugcc aucuccaugc uguaccugga | 1380 |
| ugaguaugau aaggugguac | |
| ugaaaaauua ucaggagaug guaguagagg gaugugggug | 1440 |
| ccgcugagau caggcagucc | |
| uugaggauag acagauauac acaccacaca cacacaccac | 1500 |
| auacaccaca cacacacguu | |
| cccauccacu cacccacaca cuacacagac ugcuuccuua | 1560 |
| uagcuggacu uuuauuuaaa | |
| aaaaaaaaaa aaaaaggaaa aaaucccuaa acauucaccu | 1620 |
| ugaccuuauu uaugacuuua | |
| cgugcaaaug uuuugaccau auugaucaua uauuuugaca | 1680 |
| aaauauauuu auaacuacgu | |
| auuaaaagaa aaaaauaaaa ugagucauua uuuuaaaggu | 1723 |
| aaa | |

SEQ ID NO: 22:
ucuuggugca aagaccugc
SEQ ID NO: 23:
auuucgaguu ggcuguugc
SEQ ID NO: 24:
auucucguca agguacagc
SEQ ID NO: 25:
ugcgcagaau gagaugaguu g
(hPD-1.08A heavy chain variable region):
SEQ ID NO: 26:
QVQLQQPGAE LVKPGASVKL SCKASGYTFT SYYLYWMKQR
PGQGLEWIGG VNPSNGGTNF SEKFKSKATL TVDKSSSTAY
MQLSSLTSED SAVYYCTRRD SNYDGGFDYW GQGTTLTVSS
AK
(hPD-1.08A light chain variable region):
SEQ ID NO: 27
DIVLTQSPTS LAVSLGQRAT ISCRASKSVS TSGFSYLHWY
QQKPGQPPKL LIFLASNLES GVPARFSGSG SGTDFTLNIH
PVEEEDAATY YCQHSWELPL TFGAGTKLEL K
(hPD-1.09A heavy chain variable region):
SEQ ID NO: 28
QVQLQQPGAE LVKPGTSVKL SCKASGYTFT NYYMYWVKQR
PGQGLEWIGG INPSNGGTNF NEKFKNKATL TVDSSSSTTY
MQLSSLTSED SAVYYCTRRD YRFDMGFDYW GQGTTLTVSS
AK
(hPD-1.09A light chain variable region):
SEQ ID NO: 29
DIVLTQSPAS LAVSLGQRAA ISCRASKGVS TSGYSYLHWY
QQKPGQSPKL LIYLASYLES GVPARFSGSG SGTDFTLNIH
PVEEEDAATY YCQHSRDLPL TFGTGTKLEL K
(hPD-1.08A light chain CDR1):
SEQ ID NO: 30
RASKSVSTSG FSYLH
(hPD-1.08A light chain CDR2):
SEQ ID NO: 31
LASNLES
(hPD-1-08A light chain CDR3):
SEQ ID NO: 32
QHSWELPLT
(hPD-1.08A heavy chain CDR1):
SEQ ID NO: 33
SYYLY
(hPD-1.08A heavy chain CDR2)
SEQ ID NO: 34
GVNP SNGGTN FSEKFKS
(hPD-1.08A heavy chain CDR3):
SEQ ID NO: 35
RDSNYDGGFD Y
(hPD-1.09A light chain CDR1):
SEQ ID NO: 36
RASKGVSTSG YSYLH
(hPD-1.09A light chain CDR2):
SEQ ID NO: 37
LASYLES
(hPD-1.09A light chain CDR3):
SEQ ID NO: 38
QHSRDLPLT
(hPD-1.09A heavy chain CDR1):
SEQ ID NO: 39
NYYMY
(hPD-1.09A heavy chain CDR2):
SEQ ID NO: 40
GINPSNGGTN FNEKFKN
(hPD-1,09A heavy chain CDR3):
SEQ ID NO: 41
RDYRFDMGFD Y
(109A-H heavy chain variable region):
SEQ ID NO: 42
MDWTWSILFL VAAPTGAHSQ VQLVQSGVEV KKPGASVKVS
CKASGYTFTN YYMYWVRQAP GQGLEWMGGI NPSNGGTNFN
EKFKNRVTLT TDSSTTTAYM ELKSLQFDDT AVYYCARRDY
RFDMGEDYWG QGTTVTVSS
(409A-H heavy chain full length):
SEQ ID NO: 43
MAVLGLLFCL VTFPSCVLSQ VQLVQSGVEV KKPGASVKVS
CKASGYTFTN YYMYWVRQAP GQGLEWMGGI NPSNGGTNEN
EKFKNRVTLT TDSSTTTAYM ELKSLQFDDT AVYYCARRDY
RFDMGFDYWG QGTTVTVSSA STKGPSVFPL APCSRSTSES
TAALGCLVKD YFPEPVTVSW NSGALTSGVH TFPAVLQSSG
LYSLSSVVTV PSSSLGTKTY TCNVDHKPSN TKVDKRVESK
YGPPCPPCPA PEFLGGPSVE LFPPKPKDTL MISRTPEVTC
VVVDVSQEDP EVQFNWYVDG VEVHNAKTKP REEQFNSTYR
VVSVLTVLHQ DWLNGKEYKC KVSNKGLPSS IEKTISKAKG
QPREPQVYTL PPSQEEMTKN QVSLTCLVKG FYPSDIAVEW
ESNGQPENNY KTTPPVLDSD GSFFLYSRLT VDKSRWQEGN
VFSCSVMHEA LHNHYTQKSL SLSLGK
(K09A-L-11 light chain variable region):
SEQ ID NO: 44
MAPVQLLGLL VLFLPAMRCE IVLTQSPATL SLSPGERATL
SCRASKGVST SGYSYLHWYQ QKPGQAPRLL VPARFSGSGS
GTDFTLTISS LEPEDFAVYY CQHSRDLPLT FGGGTKVEIK
(K09A-L-16 light chain variable region):
SEQ ID NO: 45
MAPVQLLGLL VLFLPAMRCE IVLTQSPLSL PVTPGEPASI
SCRASKGVST SGYSYLHWYL QKPGQSPQLL IYLASYLESG
VPDRFSGSGS GTDFTLKISR VEAEDVGVYY CQHSRDLPLT
FGQGTKLEIK
(K09A-L-17 light chain variable region):
SEQ ID NO: 46
MAPVQLLGLL VLFLPAMRCD IVMTQTPLSL PVTPGEPASI
SCRASKGVST SGYSYLHWYL QKPGQSPQLL IYLASYLESG
VPDRFSGSGS GTAFTLKISR VEAEDVGLYY CQHSRDLPLT
FGQGTKLEIK
(109A-H heavy chain full length):
SEQ ID NO: 47
MAVLGLLFCL VTFPSCVLSQ VQLVQSGVEV KKPGASVKVS
CKASGYTFTN YYMYWVRQAP GQGLEWMGGI NPSNGGTNEN
EKFKNRVTLT TDSSTTTAYM ELKSLQFDDT AVYYCARRDY
RFDMGFDYWG QGTTVTVSSA STKGPSVFPL APSSKSTSGG
TAALGCLVKD YFPEPVTVSW NSGALTSGVH TFPAVLQSSG
LYSLSSVVTV PSSSLGTQTY ICNVNHKPSN TKVDKKVEPK
SCDKTHTCPP CPAPELLGGP SVFLFPPKPK DTLMISRTPE
VTCVVVDVSH EDPEVKENWY VDGVEVHNAK TKPREEQYNS
TYRVVSVLTV LHQDWLNGKE YKCKVSNKAL PAPIEKTISK
AKGQPREPQV YTLPPSRDEL TKNQVSLTCL VKGFYPSDIA
VEWESNGQPE NNYKTTPPVL DSDGSFFLYS KLTVDKSRWQ
QGNVFSCSVM HEALHNHYTQ KSLSLSPGK
(K09A-L-11 light chain full length):
SEQ ID NO: 48
MAPVQLLGLL VLFLPAMRCE IVLTQSPATL SLSPGERATL
SGYSYLHWYQ QKPGQAPRLL VPARFSGSGS GTDFTLTISS
LEPEDFAVYY CQHSRDLPLT FGGGTKVEIK RTVAAPSVFI
FPPSDEQLKS GTASVVCLLN NFYPREAKVQ WKVDNALQSG
NSQESVTEQD SKDSTYSLSS TLTLSKADYE KHKVYACEVT
HQGLSSPVTK SENRGEC
(K09A-L-16 light chain full length):
SEQ ID NO: 49
VLFLPAMRCE IVLTQSPLSL VPDRFSGSGS GTDFTLKISR
VEAEDVGVYY CQHSRDLPLT FGQGTKLEIK RTVAAPSVFI
FPPSDEQLKS GTASVVCLLN NFYPREAKVQ WKVDNALQSG
NSQESVTEQD SKDSTYSLSS TLTLSKADYE KHKVYACEVT
HQGLSSPVTK SENRGEC
(K09A-L-17 light chain full length):
SEQ ID NO: 50
MAPVQLLGLL VLFLPAMRCD IVMTQTPLSL PVTPGEPASI
SCRASKGVST SGYSYLHWYL QKPGOSPQLL IYLASYLESG
VPDRFSGSGS GTAFTLKISR VEAEDVGLYY CQHSRDLPLT
FGQGTKLEIK RTVAAPSVFI FPPSDEQLKS GTASVVCLLN
NFYPREAKVQ WKVDNALQSG NSQESVTEQD SKDSTYSLSS
TLTLSKADYE KHKVYACEVT HQGLSSPVTK SENRGEC

1. A drug delivery system for the application to an esophageal mucous membrane, comprising
at least one sheet like preparation comprising an active pharmaceutical ingredient;
a release mechanism; and
a trigger mechanism, wherein
the trigger mechanism is adapted to trigger, at a predetermined site of action, the release of the preparation by the release mechanism, and wherein the release mechanism is adapted to release said preparation while moving along the esophageal mucous membrane,
wherein the drug delivery system further comprises a shell, wherein the shell contains the preparation, and wherein the shell comprises an aperture as part of the release mechanism configured to allow said preparation to leave the shell, and wherein the trigger mechanism is a holding device that is a part of or is attached to the preparation, such that the preparation is unrolled or unfolded while the dosage form moves down the esophageal mucous membrane and leaves the shell through the aperture,
characterized in that the active pharmaceutical ingredient comprises an agent effective in the treatment or prevention of an esophageal disease.

2. The drug delivery system of claim 1, wherein the agent effective in the treatment or prevention of an esophageal disease comprises an inhibiting polynucleotide.

3. The drug delivery system of claim 2, wherein the inhibiting polynucleotide is selected from the group consisting of a small interfering RNA (siRNA) molecule, an antisense oligonucleotide, and an aptamer.

4. The drug delivery system of claim 2, wherein the inhibiting polynucleotide comprises:
a) an siRNA molecule or an antisense oligonucleotide and targets an RNA transcript or a portion thereof encoding a BMP2 or a BMP4 polypeptide;
b) an siRNA molecule or an antisense oligonucleotide which targets an RNA transcript comprising SEQ ID NO: 20 or SEQ ID NO: 21 or a portion thereof or
c) an aptamer interfering with the activity of a BMP2 or a BMP4 polypeptide.

5. The drug delivery system of claim 3, wherein the siRNA molecule comprises:
(a) a duplex region comprising a sequence comprising SEQ ID NO: 17, SEQ ID NO: 18 or SEQ ID NO: 19, or any other sequence comprising a sequence identity of 80% or more between the siRNA molecule and the target RNA transcript or a portion thereof encoding a BMP2 or a BMP4 polypeptide,
(b) a duplex region, wherein the duplex region comprises a sense strand and an antisense strand wherein the sense strand and the antisense strand together form the duplex region, and the antisense strand is complementary to the target RNA transcript comprising SEQ ID NO: 20 or SEQ ID NO: 21 or a portion thereof, and/or

| | BMP2-siRNA 1 |
|---|---|
| | sense strand; |
| SEQ ID NO: 17 | |
| | (GCAGGUCUUUGCACCAAGA;; |
| | antisense strand |
| SEQ ID NO: 22 | |
| | UCUUGGUGCAAAGACCUGC;;), |
| | BMP2-siRNA 2 |
| | sense strand; |
| SEQ ID NO: 18 | |
| | (GCAACAGCCAACUCGAAAU;; |
| | antisaense strand |
| SEQ ID NO: 23 | |
| | AUUUCGAGUUGGCUGUUGC;;) |
| | or |
| | BMP2- siRNA 3 |
| | sense strand; |
| SEQ ID NO: 19 | |
| | (GCUGUACCUUGACGAGAAU;; |
| | antisense strand; |
| SEQ ID NO: 24 | |
| | AUUCUCGUCAAGGUACAGC;;) |

wherein each of the sequences comprises an overhang of two nucleotides dTdT (deoxythymidine) or UU (uridine) attached to the 3′ end of each strand.

6. The drug delivery system of claim 16, wherein:
i) the antibody or a binding fragment thereof binds within
a) residues 10-17, 45-56, and 69 of BMP4 (SEQ ID NO: 1),
b) residues 24-31, 57-68, 70-72, 89, 91, 101, 103, 104 and 106 of BMP4 (SEQ ID NO:1), or
c) residues 34, 35, 39, 86-88, 90, 97, 98, 100, 102 and 109 of BMP4 (SEQ ID NO:1);
ii) the antibody or a binding fragment thereof binding to at least Lys12, Arg15, Asp46, and Pro50 of BMP4 comprises a heavy chain CDR1 consisting of the amino acid sequence of SEQ ID NO: 2 or a sequence not differing more than 1 amino acid thereof, a heavy chain CDR2 consisting of the amino acid sequence of SEQ ID NO: 3, or a sequence not differing more than 1 amino acid thereof, and a heavy chain CDR3 consisting of the amino acid sequence of SEQ ID NO: 4 or a sequence not differing more than 1 amino acid thereof;
iii) the antibody or a binding fragment thereof binding to at least Asp30, Trp31, Leu66 and Lys101 of BMP4 comprises a heavy chain CDR1 consisting of the amino acid sequence of SEQ ID NO: 5 or a sequence not differing more than 1 amino acid thereof, a heavy chain CDR2 consisting of the amino acid sequence of SEQ ID NO: 6, or a sequence not differing more than 1 amino acid thereof, and a heavy chain CDR3 consisting of the amino acid sequence of SEQ ID NO: 7 or a sequence not differing more than 1 amino acid thereof;
iv) the antibody or a binding fragment thereof binding to at least Ala34, Gln39, Ser88, Leu90 and Leu100 of BMP4 comprises a heavy chain CDR1 consisting of the amino acid sequence of SEQ ID NO: 8 or a sequence not differing more than 1 amino acid thereof, a heavy chain CDR2 consisting of the amino acid sequence of SEQ ID NO: 9, or a sequence not differing more than 1 amino acid thereof, and a heavy chain CDR3 consisting of the amino acid sequence of SEQ ID NO: 10 or a sequence not differing more than 1 amino acid thereof;
iii v) the antibody or a binding fragment thereof binding to at least Lys12, Arg15, Asp46, and Pro50 of BMP4 comprises the amino acid sequence of SEQ ID NO: 11 or a sequence which is at least 70% identical thereto;
vi) the antibody or a binding fragment thereof binding to at least Asp30, Trp31, Leu66 and Lys101 of BMP4 comprises the acid sequence of SEQ ID NO: 12 or a sequence which is at least 70%; or
vii) the antibody or a binding fragment thereof binding to at least Ala34, Gln39, Ser88, Leu90 and Leu100 of BMP4 comprises the acid sequence of SEQ ID NO: 13 or a sequence which is at least 70% identical thereto.

7. The drug delivery system of claim 16, wherein the antibody comprises:
i) an antibody or antibody fragment which binds to PD-1, comprising:
a. at least one CDR (complementary determining region) selected from the group consisting of SEQ ID NOs: 30, 31, 32, 36, 37 and 38, or a variant of any said sequence; and/or
b. at least one a CDR selected from the group consisting of SEQ ID NOs: 33, 34, 35, 39, 40 and 41, or a variant of any said sequence;
ii) an antibody or antibody fragment which binds to PD-1 comprising:
a. light chain CDRs SEQ ID NOs: 30, 31 and 32, or variants of any said sequences; and/or heavy chain CDRs SEQ ID NOs: 33, 34 and 35, or variants of any said sequences; or
b. light chain CDRs SEQ ID NOs: 36, 37 and 38 or variants of any said sequences; and/or heavy chain CDRs SEQ ID NOs: 39, 40 and 41 or variants of any said sequences;
iii) an antibody or antibody fragment which binds to PD-1, comprising:
a. a heavy chain variable region comprising an amino acid sequence selected from the group consisting of:
i. SEQ ID NO: 26 or a variant thereof;
ii. SEQ ID NO: 28 or a variant thereof;
iii. amino acid residues 20 to 139 of SEQ ID NO: 42 or a variant thereof; and
iv. an amino acid sequence having at least 50% sequence identity to amino acid residues 20 to 139 of SEQ ID NO: 42;
and further comprising
b. a light chain variable region comprising an amino acid sequence selected from the group consisting of:
i. SEQ ID NO: 27 or a variant thereof;
ii. SEQ ID NO: 29 or a variant thereof;
iii. amino acid residues 20 to 130 of SEQ ID NO: 44 or a variant thereof;
iv. amino acid residues 20 to 130 of SEQ ID NO: 45 or a variant thereof;
v. amino acid residues 20 to 130 of SEQ ID NO: 46 or a variant thereof; and
vi. an amino acid sequence having at least 50% sequence identity to amino acid residues 20 to 130 of SEQ ID NO: 44, 45 or 46; and/or
iv) antibody or antibody fragment which binds to PD-1comprising:
a. a heavy chain comprising an amino acid sequence selected from the group consisting of:
i. amino acid residues 20 to 466 of SEQ ID NO: 43 or a variant thereof, and
ii. amino acid residues 20 to 469 of SEQ ID NO: 47 or a variant thereof; and
b. a light chain comprising an amino acid sequence selected from the group consisting of:
i. amino acid residues 20 to 237 of SEQ ID NO: 48 or a variant thereof;
ii. amino acid residues 20 to 237 of SEQ ID NO:49 or a variant thereof, and
iii. amino acid residues 20 to 237 of SEQ ID NO: 50 or a variant thereof.

8. The drug delivery system of claim 16 wherein the antibody comprises an antibody or antibody fragment, which:
a. binds human PD-1 with a K_{D} of about 100 pM or lower;
b. binds to human PD-1 with about the same K_{D} as an antibody having a heavy chain comprising the amino acid sequence of SEQ ID NO: 43 and a light chain comprising the amino acid sequence of SEQ ID NO: 44;
c. binds to human PD-1 with about the same K_{D} as an antibody having a heavy chain comprising the amino acid sequence of SEQ ID NO: 43 and a light chain comprising the amino acid sequence of SEQ ID NO: 45;
d. binds to human PD-1 with a k_{assoc} of about 7.5×10⁵ 1/M·s or faster;
e. binds to human PD-1 with a k_{dissoc} of about 2×10⁻⁵ 1/s or slower;
f. blocks binding of human PD-L1 or human PD-L2 to human PD-1 with an IC₅₀ of about 1 nM or lower.

9. The drug delivery system of claim 16, wherein the antiproliferative agent is selected from the group consisting of a taxane; a pyrimidine analogue, and a platinum-based agent.

10. The A method of treating or preventing an esophageal disease in a patient, comprising administering the drug delivery system of claim 1 to the patient.

11. The method of claim 10, wherein the esophageal disease, is caused or related to a defect in the immune system.

12. The method of claim 11, wherein the esophageal disease is Barrett's esophagus, esophageal stricture and/or esophageal cancer.

13. The method of claim 11; further comprising monitoring the cellular uptake of the active pharmaceutical ingredient, monitoring the route of the active pharmaceutical ingredient in a tissue or organ, or monitoring treatment success.

14. The method of claim 13, wherein the active pharmaceutical ingredient is in combination with a diagnostic marker.

15. The method of claim 13 wherein the monitoring occurs in vitro.

16. The drug delivery system of claim 1, wherein:
a) the sheet like preparation is a film shaped, foil shaped or wafer shaped preparation;
b) the drug delivery system further comprises one or more additional active pharmaceutical ingredient(s);
c) the agent effective in the treatment or prevention of an esophageal disease comprises an inhibiting polynucleotide in combination with a nucleic acid delivery system; an antibody; or an antiproliferative agent; or
d) the BMP2 or a BMP4 polypeptide, preferably a BMP2 or a BMP4 polypeptide as depicted in SEQ ID NO: 15 or SEQ ID NO: 16.

17. The drug delivery system of claim 4, wherein the BMP2 or a BMP4 polypeptide comprises SEQ ID NO:15 or SEQ ID NO:16.

18. The drug delivery system of claim 5, wherein:
a) the siRNA molecule comprises SEQ ID NO:18; or
b) the BMP2 or a BMP4 polypeptide comprises SEQ ID NO:15 or SEQ ID NO:16.

19. The drug delivery system of claim 16, wherein the antibody or antibody fragment binds to human PD-1.

20. The drug delivery system of claim 9, wherein:
a) the taxane is selected from the group consisting of paclitaxel, docetaxel, and cabazitaxel;
b) the pyrimidine analogue is a uracil analogue; or
c) the platinum-based agent is selected from the group consisting of cisplatin or a salt thereof, carboplatin or a salt thereof, nedaplatin or a salt thereof, and oxaliplatin or a salt thereof.

21. The drug delivery system of claim 20, wherein:
a) the taxane is paclitaxel; or
b) the uracil analogue is preferably 5-flurouracil or capecitabin

22. The method of claim 11, wherein the esophageal disease that is caused or related to a defect in the immune system is cancer.

23. The method of claim 22, wherein the cancer is adenocarcinoma, esophageal junction carcinoma, or squamous cell carcinoma.


SEQUENCE LISTING (50 sequences)

SEQ ID NO: 1 - protein, 114 aa, synthetic construct
KHHSQRARKKNKNCRRHSLYVDFSDVGWNDWIVAPPGYQAFYCHGDCPFPLADHLNSTNHAIVQTLVNSVNSSIPKACCVPTELSAISMLYLDEYDKVVLKNYQEMVVEGCGCR

SEQ ID NO: 2 - protein, 8 aa, synthetic construct
GRTFSTYA

SEQ ID NO: 3 - protein, 8 aa, synthetic construct
SKGGGITY

SEQ ID NO: 4 - protein, 14 aa, synthetic construct
DPVSSVAKSPVAYP

SEQ ID NO: 5 - protein, 8 aa, synthetic construct
GRTFRIND

SEQ ID NO: 6 - protein, 7 aa, synthetic construct
TSGGNTN

SEQ ID NO: 7 - protein, 14 aa, synthetic construct
DGLRFDSTRYRPFD

SEQ ID NO: 8 - protein, 8 aa, synthetic construct
GSIRGFVA

SEQ ID NO: 9 - protein, 6 aa, synthetic construct
TNGGTL

SEQ ID NO: 10 - protein, 9 aa, synthetic construct
RQIGASGYD

SEQ ID NO: 11 - protein, 124 aa, synthetic construct
EVQLVESGGGLVQAGGSLRLSCAASGRTFSTYAMGWFRQAPGKEREFVAAISKGGGITYYSDSVRGRFTISKENAKNTVYLQMNSLKPEDTAVYYCAADPVSSVAKSPVAYPYWGQGTQVTVSS

SEQ ID NO: 12 - protein, 123 aa, synthetic construct
EVQLVESGGGLVQAGGSLRLSCAASGRTFRINDMGWYRQAPGKSRDMVARITSGGNTNYADSVKGRFTISRDNAKNTVYLQMNSLKPEDTAVYYCNADGLRFDSTRYRPFDSWGQGTQVTVSS

SEQ ID NO: 13 - protein, 117 aa, synthetic construct
EVQLVESGGGLVQAGGSLRLSCAASGSIRGFVAMAWYRQAPGKQREWVATVTNGGTLYGDSVKGRFTGSRDNAKNTVYLQMNSLKPEDTAVYYCALRQIGASGYDYWGQGTQVTVSS

SEQ ID NO: 14 - protein, 114 aa, synthetic construct
QAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR

SEQ ID NO: 15 - protein, 396 aa, Homo sapiens
  REGION 1..396: note = BMP2 protein
MVAGTRCLLALLLPQVLLGGAAGLVPELGRRKFAAASSGRPSSQPSDEVLSEFELRLLSMFGLKQRPTPSRDAVVPPYMLDLYRRHSGQPGSPAPDHRLERAASRANTVRSFHHEESLEELPETSGKTTRRFFFNLSSIPTEEFITSAELQVFREQMQDALGNNSSFHHRINIYEIIKPATANSKFPVTRLLDTRLVNQNASRWESFDVTPAVMRWTAQGHANHGFVVEVAHLEEKQGVSKRHVRISRSLHQDEHSWSQIRPLLVTFGHDGKGHPLHKREKRQAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR

SEQ ID NO: 16 - protein, 408 aa, Homo sapiens
MIPGNRMLMVVLLCQVLLGGASHASLIPETGKKKVAEIQGHAGGRRSGQSHELLRDFEATLLQMFGLRRRPQPSKSAVIPDYMRDLYRLQSGEEEEEQIHSTGLEYPERPASRANTVRSFHHEEHLENIPGTSENSAFRFLFNLSSIPENEVISSAELRLFREQVDQGPDWERGFHRINIYEVMKPPAEVVPGHLITRLLDTRLVHHNVTRWETFDVSPAVLRWTREKQPNYGLAIEVTHLHQTRTHQGQHVRISRSLPQGSGNWAQLRPLLVTFGHDGRGHALTRRRRAKRSPKHHSQRARKKNKNCRRHSLYVDFSDVGWNDWIVAPPGYQAFYCHGDCPFPLADHLNSTNHAIVQTLVNSVNSSIPKACCVPTELSAISMLYLDEYDKVVLKNYQEMVVEGCGCR

SEQ ID NO: 17 - RNA, 19 nt, synthetic construct
  source 1..19: mol_type = other RNA
gcaggtctttgcaccaaga

SEQ ID NO: 18 - RNA, 19 nt, synthetic construct
  source 1..19: mol_type = other RNA
gcaacagccaactcgaaat

SEQ ID NO: 19 - RNA, 19 nt, synthetic construct
  source 1..19: mol_type = other RNA
gctgtaccttgacgagaat

SEQ ID NO: 20 - RNA, 3545 nt, Homo sapiens
  misc_feature 1..3545: note = BMP2 mRNA
  source 1..3545: mol_type = mRNA
gccgccgccgccgtcgccgccgccggagtcctcgccccgccgcgctgcgcccggctcgcgctgcgctagtcgctccgcttcccacaccccgccggggactggcagccgccgccgcacatctgccgccacagcctccgccggctacccgaacgttctcggggccagcgccgagtggatcaccggggaccgcgaggcacccgcgcgccgcagaccccgcgcgggctggagcacccggcagagcgcgccacagcgccgtggcctctgctgcccgggctgcgccagagccgcggacgggcgcgcagagcgccggggactccggagccgatccctagcgccgcgatgcggagcacctactgcaggagatcgggggcctgggacgcgctggccgaggtgtgatcggaccccaggctagccacaaagggcacttggccccagggctaggagagcgaggggagagcacagccacccgcctcggcggcccgggactcggctcgactcgccggagaatgcgcccgaggacgacggggcgccagagccgcggtgctttcaactggcgagcgcgaatgggggtgcactggagtaaggcagagtgatgcgggggggcaactcgcctggcaccgagatcgccgccgtgcccttccctggacccggcgtcgcccaggatggctgccccgagccatgggccgcggcggagctagcgcggagcgcccgaccctcgacccccgagtcccggagccggccccgcgcggggccacgcgtccctcgggcgctggttcctaaggaggacgacagcaccagcttctcctttctcccttcccttccctgccccgcactcctccccctgctcgctgttgttgtgtgtcagcacttggctggggacttcttgaacttgcagggagaataacttgcgcaccccactttgcgccggtgcctttgccccagcggagcctgcttcgccatctccgagccccaccgcccctccactcctcggccttgcccgacactgagacgctgttcccagcgtgaaaagagagactgcgcggccggcacccgggagaaggaggaggcaaagaaaaggaacggacattcggtccttgcgccaggtcctttgaccagagtttttccatgtggacgctctttcaatggacgtgtccccgcgtgcttcttagacggactgcggtctcctaaaggtcgaccatggtggccgggacccgctgtcttctagcgttgctgcttccccaggtcctcctgggcggcgcggctggcctcgttccggagctgggccgcaggaagttcgcggcggcgtcgtcgggccgcccctcatcccagccctctgacgaggtcctgagcgagttcgagttgcggctgctcagcatgttcggcctgaaacagagacccacccccagcagggacgccgtggtgcccccctacatgctagacctgtatcgcaggcactcaggtcagccgggctcacccgccccagaccaccggttggagagggcagccagccgagccaacactgtgcgcagcttccaccatgaagaatctttggaagaactaccagaaacgagtgggaaaacaacccggagattcttctttaatttaagttctatccccacggaggagtttatcacctcagcagagcttcaggttttccgagaacagatgcaagatgctttaggaaacaatagcagtttccatcaccgaattaatatttatgaaatcataaaacctgcaacagccaactcgaaattccccgtgaccagacttttggacaccaggttggtgaatcagaatgcaagcaggtgggaaagttttgatgtcacccccgctgtgatgcggtggactgcacagggacacgccaaccatggattcgtggtggaagtggcccacttggaggagaaacaaggtgtctccaagagacatgttaggataagcaggtctttgcaccaagatgaacacagctggtcacagataaggccattgctagtaacttttggccatgatggaaaagggcatcctctccacaaaagagaaaaacgtcaagccaaacacaaacagcggaaacgccttaagtccagctgtaagagacaccctttgtacgtggacttcagtgacgtggggtggaatgactggattgtggctcccccggggtatcacgccttttactgccacggagaatgcccttttcctctggctgatcatctgaactccactaatcatgccattgttcagacgttggtcaactctgttaactctaagattcctaaggcatgctgtgtcccgacagaactcagtgctatctcgatgctgtaccttgacgagaatgaaaaggttgtattaaagaactatcaggacatggttgtggagggttgtgggtgtcgctagtacagcaaaattaaatacataaatatatatatatatatatattttagaaaaaagaaaaaaacaaacaaacaaaaaaaccccaccccagttgacactttaatatttcccaatgaagactttatttatggaatggaatggaaaaaaaaacagctattttgaaaatatatttatatctacgaaaagaagttgggaaaacaaatattttaatcagagaattattccttaaagatttaaaatgtatttagttgtacattttatatgggttcaaccccagcacatgaagtataatggtcagatttattttgtatttatttactattataaccactttttaggaaaaaaatagctaatttgtatttatatgtaatcaaaagaagtatcgggtttgtacataattttccaaaaattgtagttgttttcagttgtgtgtatttaagatgaaaagtctacatggaaggttactctggcaaagtgcttagcacgtttgcttttttgcagtgctactgttgagttcacaagttcaagtccagaaaaaaaaagtggataatccactctgctgactttcaagattattatattattcaattctcaggaatgttgcagagtgattgtccaatccatgagaatttacatccttattaggtggaatatttggataagaaccagacattgctgatctattatagaaactctcctcctgccccttaatttacagaaagaataaagcaggatccatagaaataattaggaaaacgatgaacctgcaggaaagtgaatgatggtttgttgttcttctttcctaaattagtgatcccttcaaaggggctgatctggccaaagtattcaataaaacgtaagatttcttcattattgatattgtggtcatatatatttaaaattgatatctcgtggccctcatcaagggttggaaatttatttgtgttttacctttacctcatctgagagctctttattctccaaagaacccagttttctaactttttgcccaacacgcagcaaaattatgcacatcgtgttttctgcccaccctctgttctctgacctatcagcttgcttttctttccaaggttgtgtgtttgaacacatttctccaaatgttaaacctatttcagataataaatatcaaatctctggca

SEQ ID NO: 21 - RNA, 1723 nt, Homo sapiens
  source 1..1723: mol_type = mRNA
agacgcagacgcagaggtcgagcgcaggccgaaagctgttcaccgttttctcgactccggggaacatggagccattccgtagtgccatcccgagcaacgcactgctgcagcttccctgagcctttccagcaagtttgttcaagattggctgtcaagaatcatggactgttattatatgccttgttttctgtcaagacaccatgattcctggtaaccgaatgctgatggtcgttttattatgccaagtcctgctaggaggcgcgagccatgctagtttgatacctgagacggggaagaaaaaagtcgccgagattcagggccacgcgggaggacgccgctcagggcagagccatgagctcctgcgggacttcgaggcgacacttctgcagatgtttgggctgcgccgccgcccgcagcctagcaagagtgccgtcattccggactacatgcgggatctttaccggcttcagtctggggaggaggaggaagagcagatccacagcactggtcttgagtatcctgagcgcccggccagccgggccaacaccgtgaggagcttccaccacgaagaacatctggagaacatcccagggaccagtgaaaactctgcttttcgtttcctctttaacctcagcagcatccctgagaacgaggtgatctcctctgcagagcttcggctcttccgggagcaggtggaccagggccctgattgggaaaggggcttccaccgtataaacatttatgaggttatgaagcccccagcagaagtggtgcctgggcacctcatcacacgactactggacacgagactggtccaccacaatgtgacacggtgggaaacttttgatgtgagccctgcggtccttcgctggacccgggagaagcagccaaactatgggctagccattgaggtgactcacctccatcagactcggacccaccagggccagcatgtcaggattagccgatcgttacctcaagggagtgggaattgggcccagctccggcccctcctggtcacctttggccatgatggccggggccatgccttgacccgacgccggagggccaagcgtagccctaagcatcactcacagcgggccaggaagaagaataagaactgccggcgccactcgctctatgtggacttcagcgatgtgggctggaatgactggattgtggccccaccaggctaccaggccttctactgccatggggactgcccctttccactggctgaccacctcaactcaaccaaccatgccattgtgcagaccctggtcaattctgtcaattccagtatccccaaagcctgttgtgtgcccactgaactgagtgccatctccatgctgtacctggatgagtatgataaggtggtactgaaaaattatcaggagatggtagtagagggatgtgggtgccgctgagatcaggcagtccttgaggatagacagatatacacaccacacacacacaccacatacaccacacacacacgttcccatccactcacccacacactacacagactgcttccttatagctggacttttatttaaaaaaaaaaaaaaaaaaggaaaaaatccctaaacattcaccttgaccttatttatgactttacgtgcaaatgttttgaccatattgatcatatattttgacaaaatatatttataactacgtattaaaagaaaaaaataaaatgagtcattattttaaaggtaaa

SEQ ID NO: 22 - RNA, 19 nt, synthetic construct
  source 1..19: mol_type = other RNA
tcttggtgcaaagacctgc

SEQ ID NO: 23 - RNA, 19 nt, synthetic construct
  source 1..19: mol_type = other RNA
atttcgagttggctgttgc

SEQ ID NO: 24 - RNA, 19 nt, synthetic construct
  source 1..19: mol_type = other RNA
attctcgtcaaggtacagc

SEQ ID NO: 25 - RNA, 21 nt, synthetic construct
  source 1..21: mol_type = other RNA
tgcgcagaatgagatgagttg

SEQ ID NO: 26 - protein, 122 aa, synthetic construct
QVQLQQPGAELVKPGASVKLSCKASGYTFTSYYLYWMKQRPGQGLEWIGGVNPSNGGTNFSEKFKSKATLTVDKSSSTAYMQLSSLTSEDSAVYYCTRRDSNYDGGFDYWGQGTTLTVSSAK

SEQ ID NO: 27 - protein, 111 aa, synthetic construct
DIVLTQSPTSLAVSLGQRATISCRASKSVSTSGFSYLHWYQQKPGQPPKLLIFLASNLESGVPARFSGSGSGTDFTLNIHPVEEEDAATYYCQHSWELPLTFGAGTKLELK

SEQ ID NO: 28 - protein, 122 aa, synthetic construct
QVQLQQPGAELVKPGTSVKLSCKASGYTFTNYYMYWVKQRPGQGLEWIGGINPSNGGTNFNEKFKNKATLTVDSSSSTTYMQLSSLTSEDSAVYYCTRRDYRFDMGFDYWGQGTTLTVSSAK

SEQ ID NO: 29 - protein, 111 aa, synthetic construct
DIVLTQSPASLAVSLGQRAAISCRASKGVSTSGYSYLHWYQQKPGQSPKLLIYLASYLESGVPARFSGSGSGTDFTLNIHPVEEEDAATYYCQHSRDLPLTFGTGTKLELK

SEQ ID NO: 30 - protein, 15 aa, synthetic construct
RASKSVSTSGFSYLH

SEQ ID NO: 31 - protein, 7 aa, synthetic construct
LASNLES

SEQ ID NO: 32 - protein, 9 aa, synthetic construct
QHSWELPLT

SEQ ID NO: 33 - protein, 5 aa, synthetic construct
SYYLY

SEQ ID NO: 34 - protein, 17 aa, synthetic construct
GVNPSNGGTNFSEKFKS

SEQ ID NO: 35 - protein, 11 aa, synthetic construct
RDSNYDGGFDY

SEQ ID NO: 36 - protein, 15 aa, synthetic construct
RASKGVSTSGYSYLH

SEQ ID NO: 37 - protein, 7 aa, synthetic construct
LASYLES

SEQ ID NO: 38 - protein, 9 aa, synthetic construct
QHSRDLPLT

SEQ ID NO: 39 - protein, 5 aa, synthetic construct
NYYMY

SEQ ID NO: 40 - protein, 17 aa, synthetic construct
GINPSNGGTNFNEKFKN

SEQ ID NO: 41 - protein, 11 aa, synthetic construct
RDYRFDMGFDY

SEQ ID NO: 42 - protein, 139 aa, synthetic construct
  SIGNAL 1..19
MDWTWSILFLVAAPTGAHSQVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSS

SEQ ID NO: 43 - protein, 466 aa, synthetic construct
  SIGNAL 1..19
MAVLGLLFCLVTFPSCVLSQVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 44 - protein, 130 aa, synthetic construct
  SIGNAL 1..19
MAPVQLLGLLVLFLPAMRCEIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIK

SEQ ID NO: 45 - protein, 130 aa, synthetic construct
  SIGNAL 1..19
MAPVQLLGLLVLFLPAMRCEIVLTQSPLSLPVTPGEPASISCRASKGVSTSGYSYLHWYLQKPGQSPQLLIYLASYLESGVPDRFSGSGSGTDFTLKISRVEAEDVGVYYCQHSRDLPLTFGQGTKLEIK

SEQ ID NO: 46 - protein, 130 aa, synthetic construct
  SIGNAL 1..19
MAPVQLLGLLVLFLPAMRCDIVMTQTPLSLPVTPGEPASISCRASKGVSTSGYSYLHWYLQKPGQSPQLLIYLASYLESGVPDRFSGSGSGTAFTLKISRVEAEDVGLYYCQHSRDLPLTFGQGTKLEIK

SEQ ID NO: 47 - protein, 469 aa, synthetic construct
  SIGNAL 1..19
MAVLGLLFCLVTFPSCVLSQVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 48 - protein, 237 aa, synthetic construct
  SIGNAL 1..19
MAPVQLLGLLVLFLPAMRCEIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 49 - protein, 237 aa, synthetic construct
  SIGNAL 1..19
MAPVQLLGLLVLFLPAMRCEIVLTQSPLSLPVTPGEPASISCRASKGVSTSGYSYLHWYLQKPGQSPQLLIYLASYLESGVPDRFSGSGSGTDFTLKISRVEAEDVGVYYCQHSRDLPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 50 - protein, 237 aa, synthetic construct
  SIGNAL 1..19
MAPVQLLGLLVLFLPAMRCDIVMTQTPLSLPVTPGEPASISCRASKGVSTSGYSYLHWYLQKPGQSPQLLIYLASYLESGVPDRFSGSGSGTAFTLKISRVEAEDVGLYYCQHSRDLPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC